SEQUENCE LISTING (40 sequences)

SEQ ID NO: 1 - protein, 1119 aa, Homo sapiens
MKRSLRKMWRPGEKKEPQGVVYEDVPDDTEDFKESLKVVFEGSAYGLQNFNKQKKLKRCDDMDTFFLHYAAAEGQIELMEKITRDSSLEVLHEMDDYGNTPLHCAVEKNQIESVKFLLSRGANPNLRNFNMMAPLHIAVQGMNNEVMKVLLEHRTIDVNLEGENGNTAVIIACTTNNSEALQILLKKGAKPCKSNKWGCFPIHQAAFSGSKECMEIILRFGEEHGYSRQLHINFMNNGKATPLHLAVQNGDLEMIKMCLDNGAQIDPVEKGRCTAIHFAATQGATEIVKLMISSYSGSVDIVNTTDGCHETMLHRASLFDHHELADYLISVGADINKIDSEGRSPLILATASASWNIVNLLLSKGAQVDIKDNFGRNFLHLTVQQPYGLKNLRPEFMQMQQIKELVMDEDNDGCTPLHYACRQGGPGSVNNLLGFNVSIHSKSKDKKSPLHFAASYGRINTCQRLLQDISDTRLLNEGDLHGMTPLHLAAKNGHDKVVQLLLKKGALFLSDHNGWTALHHASMGGYTQTMKVILDTNLKCTDRLDEDGNTALHFAAREGHAKAVALLLSHNADIVLNKQQASFLHLALHNKRKEVVLTIIRSKRWDECLKIFSHNSPGNKCPITEMIEYLPECMKVLLDFCMLHSTEDKSCRDYYIEYNFKYLQCPLEFTKKTPTQDVIYEPLTALNAMVQNNRIELLNHPVCKEYLLMKWLAYGFRAHMMNLGSYCLGLIPMTILVVNIKPGMAFNSTGIINETSDHSEILDTTNSYLIKTCMILVFLSSIFGYCKEAGQIFQQKRNYFMDISNVLEWIIYTTGIIFVLPLFVEIPAHLQWQCGAIAVYFYWMNFLLYLQRFENCGIFIVMLEVILKTLLRSTVVFIFLLLAFGLSFYILLNLQDPFSSPLLSIIQTFSMMLGDINYRESFLEPYLRNELAHPVLSFAQLVSFTIFVPIVLMNLLIGLAVGDIAEVQKHASLKRIAMQVELHTSLEKKLPLWFLRKVDQKSTIVYPNKPRSGGMLFHIFCFLFCTGEIRQEIPNADKSLEMEILKQKYRLKDLTFLLEKQHELIKLIIQKMEIISETEDDDSHCSFQDRFKKEQMEQRNSRWNTVLRAVKAKTHHLEP

SEQ ID NO: 2 - protein, 1119 aa, Homo sapiens; identical to SEQ ID NO: 1

SEQ ID NO: 3 - DNA, 5191 bp, Homo sapiens
ccagaagttctccagggcttccgcagagcgactttttcgctgcctgtgagctgcagcgcgggagagctcgggctcgcgcggaccccagcgcctggcaggctgacagcgctctctcgccccaggtgcccgcgcgcgtggtgagcagctgcaccaggtggcgtccggggtggggtcaatgaagcgcagcctgaggaagatgtggcgccctggagaaaagaaggagccccagggcgttgtctatgaggatgtgccggacgacacggaggatttcaaggaatcgcttaaggtggtttttgaaggaagtgcatatggattacaaaactttaataagcaaaagaaattaaaaagatgtgacgatatggacaccttcttcttgcattatgctgcagcagaaggccaaattgagctaatggagaagatcaccagagattcctctttggaagtgctgcatgaaatggatgattatggaaatacccctctgcattgtgctgtagaaaaaaaccaaattgaaagcgttaagtttcttctcagcagaggagcaaacccaaatctccgaaacttcaacatgatggctcctctccacatagctgtgcagggcatgaataatgaggtgatgaaggtcttgcttgagcatagaactattgatgttaatttggaaggagaaaatggaaacacagctgtgatcattgcgtgcaccacaaataatagcgaagcattgcagattttgcttaaaaaaggagctaagccatgtaaatcaaataaatggggatgtttccctattcaccaagctgcattttcaggttccaaagaatgcatggaaataatactaaggtttggtgaagagcatgggtacagtagacagttgcacattaactttatgaataatgggaaagccacccctctccacctggctgtgcaaaatggtgacttggaaatgatcaaaatgtgcctggacaatggtgcacaaatagacccagtggagaagggaaggtgcacagccattcattttgctgccacccagggagccactgagattgttaaactgatgatatcgtcctattctggtagcgtggatattgttaacacaaccgatggatgtcatgagaccatgcttcacagagcttcattgtttgatcaccatgagctagcagactatttaatttcagtgggagcagatattaataagatcgattctgaaggacgctctccacttatattagcaactgcttctgcatcttggaatattgtaaatttgctactctctaaaggtgcccaagtagacataaaagataattttggacgtaattttctgcatttaactgtacagcaaccttatggattaaaaaatctgcgacctgaatttatgcagatgcaacagatcaaagagctggtaatggatgaagacaacgatgggtgtactcctctacattatgcatgtagacaggggggccctggttctgtaaataacctacttggctttaatgtgtccattcattccaaaagcaaagataagaaatcacctctgcattttgcagccagttatgggcgtatcaatacctgtcagaggctcctacaagacataagtgatacgaggcttctgaatgaaggtgaccttcatggaatgactcctctccatctggcagcaaagaatggacatgataaagtagttcagcttcttctgaaaaaaggtgcattgtttctcagtgaccacaatggctggacagctttgcatcatgcgtccatgggcgggtacactcagaccatgaaggtcattcttgatactaatttgaagtgcacagatcgcctggatgaagacgggaacactgcacttcactttgctgcaagggaaggccacgccaaagccgttgcgcttcttctgagccacaatgctgacatagtcctgaacaagcagcaggcctcctttttgcaccttgcacttcacaataagaggaaggaggttgttcttacgatcatcaggagcaaaagatgggatgaatgtcttaagattttcagtcataattctccaggcaataaatgtccaattacagaaatgatagaatacctccctgaatgcatgaaggtacttttagatttctgcatgttgcattccacagaagacaagtcctgccgagactattatatcgagtataatttcaaatatcttcaatgtccattagaattcaccaaaaaaacacctacacaggatgttatatatgaaccgcttacagccctcaacgcaatggtacaaaataaccgcatagagcttctcaatcatcctgtgtgtaaagaatatttactcatgaaatggttggcttatggatttagagctcatatgatgaatttaggatcttactgtcttggtctcatacctatgaccattctcgttgtcaatataaaaccaggaatggctttcaactcaactggcatcatcaatgaaactagtgatcattcagaaatactagataccacgaattcatatctaataaaaacttgtatgattttagtgtttttatcaagtatatttgggtattgcaaagaagcggggcaaattttccaacagaaaaggaattattttatggatataagcaatgttcttgaatggattatctacacgacgggcatcatttttgtgctgcccttgtttgttgaaataccagctcatctgcagtggcaatgtggagcaattgctgtttacttctattggatgaatttcttattgtatcttcaaagatttgaaaattgtggaatttttattgttatgttggaggtaattttgaaaactttgttgaggtctacagttgtatttatcttccttcttctggcttttggactcagcttttacatcctcctgaatttacaggatcccttcagctctccattgctttctataatccagaccttcagcatgatgctaggagatatcaattatcgagagtccttcctagaaccatatctgagaaatgaattggcacatccagttctgtcctttgcacaacttgtttccttcacaatatttgtcccaattgtcctcatgaatttacttattggtttggcagttggcgacattgctgaggtccagaaacatgcatcattgaagaggatagctatgcaggtggaacttcataccagcttagagaagaagctgccactttggtttctacgcaaagtggatcagaaatccaccatcgtgtatcccaacaaacccagatctggtgggatgttattccatatattctgttttttattttgcactggggaaataagacaagaaataccaaatgctgataaatctttagaaatggaaatattaaagcagaaataccggctgaaggatcttacttttctcctggaaaaacagcatgagctcattaaactgatcattcagaagatggagatcatctctgagacagaggatgatgatagccattgttcttttcaagacaggtttaagaaagagcagatggaacaaaggaatagcagatggaatactgtgttgagagcagtcaaggcaaaaacacaccatcttgagccttagctcctcagaccttcagtgaggcttctaatggggggtgcatgacttgctggttctaactttcaatttaaaaagagtgaggaagaagcagaatgattcattttgctgcgtgtgaaatcatggttcctgcatgctgtataaaagtaaaccatcttttatcctctattcatattttctaccaatcactatgtattggggatatctttgcagatatgttcaaattggactggactttgatgagatataatctcattatttgaatgggtagaaaatgaatttgctagaacacacatttttaatgaaaagaagtaataaatgtaactattaagctaaaatgcaaatgtcagtactgaattcctgcttgttaattacataatatgtgatgctctagaaaatagtcacaagtattaataatgccttagatgatagtcttaaatattaggttgaggtctacctaacctaagctgcttcctggaaagcttcatgttgaaagaacctatgggtggcaccatgtggacttttctgtccctactgtgatgaatagccccacccttcttgctgtccccaacacacctgatgtcactttgagccatatagttgaagtacaaattaataggccttatgatatgcacgaattttactatagataatatatgttgtttctggttttgtttgccaatgagcataataaatgtaaaacctatatagtatccctgtgattattgtatgagcctttgtttgagatttgaaaacaacatggctccatcacatattcccttttttcttttgatgtctactcaaatcatgaattaatcacatacctcatcattaatcttttcaaggtccttctattgttttgtctgattttctccatcatcctgattagcatgtttattccctcactacccccaggagatattcactgtaatgaatatgtctttggctatgtatgtgtccttgtgttatgttgtacagtgttgttttgagtctgttattatttacacagatgttattatgctatagcttctatttctgtttttgcttcttatttctcttataattctcacttatttcctattttttctactcatttctatttgttactcctttttactggacatgatgtttacaagatacaactgtgttactgtattccatctagtacggggcctttggtgtggcttactatttcattgtgtgcacccacccacccaccacactggacttttctagagatggacagcttggttacctccaccttcctgcactcattctcaaacatactgatgttcatacaaaccagcagagtgctgagggacgatatgtactattacaaaaccagacacttttacattcatggtccaacagatcacatggcctagaggcaatgttgcatataccttaatctttgatatgaataatatctttgttctttatatttcttaaaacagaaagggtggaaaatcactatacagaagcaatatccaaagatctcctgatcataaagacaaggggtcttttcagtcttccctctcctcaaaccttgtgtagcattgcacaatatagatctcagtcaacattcactgagtgccaagaatgtgagaaacactgtaccatgcctgtcatgcgaaatatttaaataaacagattgtcttacaa

SEQ ID NO: 4 - protein, 1119 aa, Homo sapiens; identical to SEQ ID NO: 1

SEQ ID NO: 5 - protein, 1119 aa, Artificial Sequence
  Description of Artificial Sequence Synthetic polypeptide
MKRSLRKMWRPGEKKEPQGVVYEDVPDDTEDFKESLKVVFEGSAYGLQNFNKQKKLKRCDDMDTFFLHYAAAEGQIELMEKITRDSSLEVLHEMDDYGNTPLHCAVEKNQIESVKFLLSRGANPNLRNFNMMAPLHIAVQGMNNEVMKVLLEHRTIDVNLEGENGNTAVIIACTTNNSEALQILLKKGAKPCKSNKWGCFPIHQAAFSGSKECMEIILRFGEEHGYSRQLHINFMNNGKATPLHLAVQNGDLEMIKMCLDNGAQIDPVEKGRCTAIHFAATQGATEIVKLMISSYSGSVDIVNTTDGCHETMLHRASLFDHHELADYLISVGADINKIDSEGRSPLILATASASWNIVNLLLSKGAQVDIKDNFGRNFLHLTVQQPYGLKNLRPEFMQMQQIKELVMDEDNDGCTPLHYACRQGGPGSVNNLLGFNVSIHSKSKDKKSPLHFAASYGRINTCQRLLQDISDTRLLNEGDLHGMTPLHLAAKNGHDKVVQLLLKKGALFLSDHNGWTALHHASMGGYTQTMKVILDTNLKCTDRLDEDGNTALHFAAREGHAKAVALLLSHNADIVLNKQQASFLHLALHNKRKEVVLTIIRSKRWDECLKIFSHNSPGNKCPITEMIEYLPECMKVLLDFCMLHSTEDKSCRDYYIEYNFKYLQCPLEFTKKTPTQDVIYEPLTALNAMVQNNRIELLNHPVCKEYLLMKWLAYGFRAHMMNLGSYCLGLIPMTILVVNIKPGMAFNSTGIINETSDHSEILDTTNSYLIKTCMILVFLSSIFGYCKEAGQIFQQKRNYFMDISNVLEWIIYTTGIIFVLPLFVEIPAHLQWQCGAIAVYFYWMNFLLYLQRFENCGIFIVMLEVILKTLLRSTVVFIFLLLAFGLSFYILLNLQDPFSSPLLSIIQTFSMMLGDINYRESFLHPYLRNELAHPVLSFAQLVSFTIFVPIVLMNLLIGLAVGDIAEVQKHASLKRIAMQVELHTSLEKKLPLWFLRKVDQKSTIVYPNKPRSGGMLFHIFCFLFCTGEIRQEIPNADKSLEMEILKQKYRLKDLTFLLEKQHELIKLIIQKMEIISETEDDDSHCSFQDRFKKEQMEQRNSRWNTVLRAVKAKTHHLEP

SEQ ID NO: 6 - protein, 1105 aa, Artificial Sequence
  Description of Artificial Sequence Synthetic polypeptide
MKRSLRKMWRPGEKKEPQGVVYEDVPDDTEDFKESLKVVFEGSAYGLQNFNKQKKLKRCDDMDTFFLHYAAAEGQIELMEKITRDSSLEVLHEMDDYGNTPLHCAVEKNQIESVKFLLSRGANPNLRNFNMMAPLHIAVQGMNNEVMKVLLEHRTIDVNLEGENGNTAVIIACTTNNSEALQILLKKGAKPCKSNKWGCFPIHQAAFSGSKECMEIILRFGEEHGYSRQLHINFMNNGKATPLHLAVQNGDLEMIKMCLDNGAQIDPVEKGRCTAIHFAATQGATEIVKLMISSYSGSVDIVNTTDGCHETMLHRASLFDHHELADYLISVGADINKIDSEGRSPLILATASASWNIVNLLLSKGAQVDIKDNFGRNFLHLTVQQPYGLKNLRPEFMQMQQIKELVMDEDNDGCTPLHYACRQGGPGSVNNLLGFNVSIHSKSKDKKSPLHFAASYGRINTCQRLLQDISDTRLLNEGDLHGMTPLHLAAKNGHDKVVQLLLKKGALFLSDHNGWTALHHASMGGYTQTMKVILDTNLKCTDRLDEDGNTALHFAAREGHAKAVALLLSHNADIVLNKQQASFLHLALHNKRKEVVLTIIRSKRWDECLKIFSHNSPGNKCPITEMIEYLPECMKVLLDFCMLHSTEDKSCRDYYIEYNFKYLQCPLEFTKKTPTQDVIYEPLTALNAMVQNNRIELLNHPVCKEYLLMKWLAYGFRAHMMNLGSYCLGLIPMTILVVNIKPGMAFNSTGIINETSDHSEILDTTNSYLIKTCMILVFLSSIFGYCKEAGQIFQQKRNYFMDISNVLEWIIYTTGIIFVLPLFVEIPAHLQWQCGAIAVYFYWMNFLLYLQRFENCGIFIVMLEVILKTLLRSTVVFIFLLLAFGLSFYILLNLQDPFSSPLLSIIQTFSMMLGDINYRESFLEPYLRNELAHPVLSFAQLVSFTIFVPIVLMNLLIGLAVGDIAEVQKHASLKRIAMQVELHTSLEKKLPLWFLRKVDQKSTIVYPNKPRSGGMLFHIFCFLFCTGEIRQEIPNADKSLEMEILKQKYRLKDLTFLLEKQHELIKLIIQKMEIISETEDDDSHCSFQDRFKKEQMEQRFCYENE

SEQ ID NO: 7 - protein, 1096 aa, Artificial Sequence
  Description of Artificial Sequence Synthetic polypeptide
MKRSLRKMWRPGEKKEPQGVVYEDVPDDTEDFKESLKVVFEGSAYGLQNFNKQKKLKRCDDMDTFFDYGNTPLHCAVEKNQIESVKFLLSRGANPNLRNFNMMAPLHIAVQGMNNEVMKVLLEHRTIDVNLEGENGNTAVIIACTTNNSEALQILLKKGAKPCKSNKWGCFPIHQAAFSGSKECMEIILRFGEEHGYSRQLHINFMNNGKATPLHLAVQNGDLEMIKMCLDNGAQIDPVEKGRCTAIHFAATQGATEIVKLMISSYSGSVDIVNTTDGCHETMLHRASLFDHHELADYLISVGADINKIDSEGRSPLILATASASWNIVNLLLSKGAQVDIKDNFGRNFLHLTVQQPYGLKNLRPEFMQMQQIKELVMDEDNDGCTPLHYACRQGGPGSVNNLLGFNVSIHSKSKDKKSPLHFAASYGRINTCQRLLQDISDTRLLNEGDLHGMTPLHLAAKNGHDKVVQLLLKKGALFLSDHNGWTALHHASMGGYTQTMKVILDTNLKCTDRLDEDGNTALHFAAREGHAKAVALLLSHNADIVLNKQQASFLHLALHNKRKEVVLTIIRSKRWDECLKIFSHNSPGNKCPITEMIEYLPECMKVLLDFCMLHSTEDKSCRDYYIEYNFKYLQCPLEFTKKTPTQDVIYEPLTALNAMVQNNRIELLNHPVCKEYLLMKWLAYGFRAHMMNLGSYCLGLIPMTILVVNIKPGMAFNSTGIINETSDHSEILDTTNSYLIKTCMILVFLSSIFGYCKEAGQIFQQKRNYFMDISNVLEWIIYTTGIIFVLPLFVEIPAHLQWQCGAIAVYFYWMNFLLYLQRFENCGIFIVMLEVILKTLLRSTVVFIFLLLAFGLSFYILLNLQDPFSSPLLSIIQTFSMMLGDINYRESFLEPYLRNELAHPVLSFAQLVSFTIFVPIVLMNLLIGLAVGDIAEVQKHASLKRIAMQVELHTSLEKKLPLWFLRKVDQKSTIVYPNKPRSGGMLFHIFCFLFCTGEIRQEIPNADKSLEMEILKQKYRLKDLTFLLEKQHELIKLIIQKMEIISETEDDDSHCSFQDRFKKEQMEQRNSRWNTVLRAVKAKTHHLEPFCYENE

SEQ ID NO: 8 - DNA, 137 bp, Mus musculus
gtgatttttaaaacattgctgagatcgaccggagtgtttatcttcctcctactggcttttggcctcagcttttatgttctcctgaatttccaagatgccttcagcaccccattgctttccttaatccagacattcag

SEQ ID NO: 9 - protein, 25 aa, Artificial Sequence
  Description of Artificial Sequence Synthetic peptide
  MOD_RES (7)..(7): K or R
  MOD_RES (12)..(12): G, E, or A
MKRSLRXMLRPXEKKEPQGVVYQGV

SEQ ID NO: 10 - protein, 25 aa, Homo sapiens
MKRSLRKMWRPGEKKEPQGVVYEDV

SEQ ID NO: 11 - protein, 25 aa, Mus sp.
MKRGLRRILLPEERKEVQGVVYRGV

SEQ ID NO: 12 - protein, 25 aa, Castor sp.
MKRSLRKMLRPAEKKEPQGVNYQGV

SEQ ID NO: 13 - protein, 25 aa, Vicugna sp.
MKRSLRKMLRPGEKKEPQGVVYQGV

SEQ ID NO: 14 - protein, 25 aa, Equus sp.
MKRSLRKMLRPGEKKEPPGVVYQGV

SEQ ID NO: 15 - protein, 25 aa, Unknown
  Description of Unknown Bat sequence
MRRSLKRMLRTAGKKEPQGVVYQGV

SEQ ID NO: 16 - protein, 24 aa, Artificial Sequence
  Description of Artificial Sequence Synthetic peptide
  MOD_RES (2)..(2): K, R, N, or Q
  MOD_RES (3)..(3): R or F
  MOD_RES (4)..(4): S or absent
  MOD_RES (5)..(5): L, I, or absent
  MOD_RES (6)..(6): R, L, or absent
  MOD_RES (7)..(7): K or absent
  MOD_RES (8)..(8): M, L, W, or absent
  MOD_RES (9)..(9): W, F, or absent
  MOD_RES (10)..(10): R, Q, or absent
  MOD_RES (11)..(11): E, G, or S
  MOD_RES (13)..(13): K, E, or D
  MOD_RES (14)..(14): E, K, V, or L
  MOD_RES (15)..(15): P, A, K, or V
  MOD_RES (16)..(16): Q, A, or R
  MOD_RES (17)..(17): G, D, S, Q, or R
  MOD_RES (18)..(18): P, T, N, or absent
  MOD_RES (19)..(19): V, D, T, or S
  MOD_RES (20)..(20): V, T, S, C, or F
  MOD_RES (22)..(22): E, H, T, or K
  MOD_RES (23)..(23): D, G, Y, or C
MXXXXXXXXXXRXXXXXXXXYXXV

SEQ ID NO: 17 - protein, 24 aa, Unknown
  Description of Unknown Alligator sequence
MRRSLLKLFQGREKAADPDTYHGV

SEQ ID NO: 18 - protein, 23 aa, Unknown
  Description of Unknown Snake sequence
MKRSILKWFQSRDEKQSTSYEGV

SEQ ID NO: 19 - protein, 17 aa, Poecilia sp.
MNFSRDVARQTSCYTYV

SEQ ID NO: 20 - protein, 17 aa, Unknown
  Description of Unknown Zebrafish sequence
MQFGKELVRRNSFYKCV

SEQ ID NO: 21 - protein, 25 aa, Homo sapiens
TNSYLIKTCMILVFLSSIFGYCKEA

SEQ ID NO: 22 - protein, 25 aa, Mus sp.
LNSFPIKICMILVFLSSIFGYCKEV

SEQ ID NO: 23 - protein, 25 aa, Castor sp.
TNSYPIKVCMVLVFLSSIFGYCKEV

SEQ ID NO: 24 - protein, 25 aa, Vicugna sp.
KNSYTIKVCMILVLLSSIFGYCKEV

SEQ ID NO: 25 - protein, 25 aa, Equus sp.
KNSYSIKVCMILVFLSSIFGYCKEV

SEQ ID NO: 26 - protein, 25 aa, Unknown
  Description of Unknown Bat sequence
KDSYSIKVCMILVFLSSLFGYCKEL

SEQ ID NO: 27 - protein, 25 aa, Unknown
  Description of Unknown Alligator sequence
EESYFIRVCMCLVLIMSLLGICKEI

SEQ ID NO: 28 - protein, 25 aa, Unknown
  Description of Unknown Snake sequence
KDSNFIKVCMSLVFIMSLFGICKEI

SEQ ID NO: 29 - protein, 25 aa, Poecilia sp.
EQSMFLSFCMVMVLITNVYSIGKEV

SEQ ID NO: 30 - protein, 25 aa, Unknown
  Description of Unknown Zebrafish sequence
EQQYLISVCIIMVIVMNVYSICKEV

SEQ ID NO: 31 - protein, 31 aa, Homo sapiens
MDTFFLHYAAAEGQIELMEKITRDSSLEVLH

SEQ ID NO: 32 - protein, 6 aa, Mus sp.
AAAEGQ

SEQ ID NO: 33 - protein, 4 aa, Mus sp.
ELME

SEQ ID NO: 34 - protein, 6 aa, Castor sp.
AAAEGQ

SEQ ID NO: 35 - protein, 4 aa, Vicugna sp.
ELME

SEQ ID NO: 36 - protein, 5 aa, Unknown
  Description of Unknown Bat sequence
LHYAA

SEQ ID NO: 37 - protein, 6 aa, Unknown
  Description of Unknown Alligator sequence
GQIELM

SEQ ID NO: 38 - protein, 4 aa, Unknown
  Description of Unknown Zebrafish sequence
LHYA

SEQ ID NO: 39 - protein, 159 aa, Homo sapiens
PLLSIIQTFSMMLGDINYRESFLEPYLRNELAHPVLSFAQLVSFTIFVPIVLMNLLIGLADMDTFFLHYAAAEGQIELMEKITRDSSLEVLHEMDDYGNTPLHCAVEKNQIESVKFLLSRDDSHCSFQDRFKKEQMEQRNSRWNTVLRAVKAKTHHLEP

SEQ ID NO: 40 - protein, 116 aa, Artificial Sequence
  Description of Artificial Sequence Synthetic polypeptide
PLLSIIQTFSMMLGDINYRESFLHPYLRNELAHPVLSFAQLVSFTIFVPIVLMNLLIGLADMDTFFDYGNTPLHCAVEKNQIESVKFLLSRDDSHCSFQDRFKKEQMEQRFCYENE

(19) United States
(12) Patent Application Publication
(10) Pub. No.: US 2023/0263891 A1
(43) Pub. Date: Aug. 24, 2023

(54) SONOGENETIC STIMULATION OF CELLS EXPRESSING TRPA1

(71) Applicant: Salk Institute for Biological Studies, La Jolla, CA (US)

(72) Inventors: Sreekanth CHALASANI, La Jolla, CA (US); Yusuf TUFAIL, La Jolla, CA (US); Jose Mendoza LOPEZ, La Jolla, CA (US); Marc Duque RAMIREZ, La Jolla, CA (US); Uri MAGARAM, La Jolla, CA (US); Corinne LEE-KUBLI, La Jolla, CA (US); Eric Warren EDSINGER, La Jolla, CA (US)

(73) Assignee: Salk Institute for Biological Studies, La Jolla, CA (US)

(21) Appl. No.: 18/016,251

(22) PCT Filed: Jul. 15, 2021
(86) PCT No.: PCT/US2021/041814

§ 371 Date: Jan. 13, 2023

Related U.S. Application Data
(60) Provisional application No. 63/053,418, filed on Jul. 17, 2020.

(51) Int. Cl. A61K 41/00; A61K 38/17; A61K 48/00; A61P 25/00; C12N 15/86

(52) U.S. Cl. CPC A61K 41/0028; A61K 38/177; A61K 48/0083; A61P 25/00; C12N 15/86; C12N 2750/14143

(57) ABSTRACT

Described herein are compositions featuring TRPA1 polypeptides and polynucleotides, methods for expressing such polypeptides and polynucleotides in a cell type of interest, and methods for inducing the activation of the TRPA1 polypeptide in neurons and other cell types using ultrasound.

### CROSS REFERENCE TO RELATED APPLICATION

This application claims priority to and benefit of U.S. Provisional Application No. 63/053,418, filed on Jul. 17, 2020, the contents of which are incorporated by reference herein in their entirety.

### STATEMENT OF RIGHTS TO INVENTIONS MADE UNDER FEDERALLY SPONSORED RESEARCH

This work was supported by the following grants from the National Institutes of Health, Grant Nos: NIH R01MH111534 and R01NS115591. The government has certain rights in the invention.

### BACKGROUND

One approach to understanding a biological system is to identify its constituent components, explore their interactions and signals, and systematically alter the inputs and/or perturb these components while monitoring the outputs. In the nervous system, these perturbations have been aided by the discovery of light-, or small molecule-sensitive proteins and their variants that can be expressed in specific cells, thereby enabling manipulation by light (optogenetics) or small molecules (chemogenetics), respectively. While light can be shaped, patterned and localized, the opacity of biological tissues makes it difficult to target deep structures in mammals without invasive surgical procedures. Likewise, small molecules can be easily delivered to various targets, but they lack the temporal precision needed to perturb cellular function on the timescales of neuronal signalling. Additionally, studies evaluating magnetic fields (magnetogenetics) for this purpose have been controversial and may similarly lack the necessary temporal precision. These reports emphasize the need for a new modality that can be used to non-invasively manipulate specific cells with millisecond precision.

Ultrasound is safe, non-invasive and can be focused easily through thin bone and tissue to volumes of a few cubic millimeters. Moreover, continuous or repeated pulses of ultrasound at frequencies between 250 KHz-3 MHz have been shown to stimulate neurons within both rodent and non-human primates. Ultrasound has also been used to safely manipulate deep nerve structures in human hands to relieve chronic pain, as well as to elicit somatosensory and visual cortex sensations through the intact human skull. These and other studies have revealed a wide interest in adapting ultrasound for both research and therapeutic purpose. The mechanisms that underlie ultrasound neurostimulation remain unclear and may include mechanical forces, heating, cavitation and astrocyte signals in vitro, or indirect auditory signals within the rodent brain. While the involvement of mechanosensitive channels in ultrasound responses of rodent neurons in vitro and C. elegans neurons in vivo has been reported, the identification of other and new channel proteins would allow for the development of broadly usable tools that would provide exogenous proteins, which render target cells selectively sensitive to ultrasound stimuli (sonogenetics).

### SUMMARY OF THE DISCLOSURE AND EMBODIMENTS

Provided and described herein are compositions featuring human TRPA1 polypeptides and polynucleotides, methods for expressing such polypeptides and polynucleotides in a cell type of interest. Methods for inducing the activation of the TRPA1 polypeptide in neurons and other cell types using ultrasound are also provided. In an aspect, a method of stimulating a cell is provided, in which the method comprises contacting a TRPA1 polypeptide expressing cell with ultrasound, thereby stimulating the cell. In an embodiment of the method, the TRPA1 polypeptide has at least about 85% identity to a TRPA1 polypeptide having the sequence of NCBI Reference Sequence: XP_016869435.1 (SEQ ID NO: 1). In an embodiment of the method, the TRPA1 polypeptide has at least 85% identity to a TRPA1 polypeptide having the following sequence:

(SEQ ID NO: 4)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQQ
PYGLKNLRPEFMQMQQIKELVMDEDNDGCTPLHYA
CRQGGPGSVNNLLGFNVSIHSKSKDKKSPLHFAAS
YGRINTCQRLLQDISDTRLLNEGDLHGMTPLHLAA
KNGHDKVVQLLLKKGALFLSDHNGWTALHHASMGG
YTQTMKVILDTNLKCTDRLDEDGNTALHFAAREGH
AKAVALLLSHNADIVLNKQQASFLHLALHNKRKEV
VLTIIRSKRWDECLKIFSHNSPGNKCPITEMIEYL
PECMKVLLDFCMLHSTEDKSCRDYYIEYNFKYLQC
PLEFTKKTPTQDVIYEPLTALNAMVQNNRIELLNH
PVCKEYLLMKWLAYGFRAHMMNLGSYCLGLIPMTI
LVVNIKPGMAFNSTGIINETSDHSEILDTTNSYLI
KTCMILVFLSSIFGYCKEAGQIFQQKRNYFMDISN
VLEWIIYTTGIIFVLPLFVEIPAHLQWQCGAIAVY
FYWMNFLLYLQRFENCGIFIVMLEVILKTLLRSTV
VFIFLLLAFGLSFYILLNLQDPFSSPLLSIIQTFS
MMLGDINYRESFLEPYLRNELAHPVLSFAQLVSFT
IFVPIVLMNLLIGLAVGDIAEVQKHASLKRIAMQV
ELHTSLEKKLPLWFLRKVDQKSTIVYPNKPRSGGM
LFHIFCFLFCTGEIRQEIPNADKSLEMEILKQKYR
LKDLTFLLEKQHELIKLIIQKMEIISETEDDDSHC
SFQDRFKKEQMEQRNSRWNTVLRAVKAKTHHLEP.

In an embodiment of the method, the TRPA1 polypeptide has at least 85% identity to a TRPA1 polypeptide having the following sequence:

(SEQ ID NO. 5)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQQ
PYGLKNLRPEFMQMQQIKELVMDEDNDGCTPLHYA
CRQGGPGSVNNLLGFNVSIHSKSKDKKSPLHFAAS
YGRINTCQRLLQDISDTRLLNEGDLHGMTPLHLAA
KNGHDKVVQLLLKKGALFLSDHNGWTALHHASMGG
YTQTMKVILDTNLKCTDRLDEDGNTALHFAAREGH
AKAVALLLSHNADIVLNKQQASFLHLALHNKRKEV
VLTIIRSKRWDECLKIFSHNSPGNKCPITEMIEYL
PECMKVLLDFCMLHSTEDKSCRDYYIEYNFKYLQC
PLEFTKKTPTQDVIYEPLTALNAMVQNNRIELLNH
PVCKEYLLMKWLAYGFRAHMMNLGSYCLGLIPMTI
LVVNIKPGMAFNSTGIINETSDHSEILDTTNSYLI
KTCMILVFLSSIFGYCKEAGQIFQQKRNYFMDISN
VLEWIIYTTGIIFVLPLFVEIPAHLQWQCGAIAVY
FYWMNFLLYLQRFENCGIFIVMLEVILKTLLRSTV
VFIFLLLAFGLSFYILLNLQDPFSSPLLSIIQTFS
MMLGDINYRESFLAPYLRNELAHPVLSFAQLVSFT
IFVPIVLMNLLIGLAVGDIAEVQKHASLKRIAMQV
ELHTSLEKKLPLWFLRKVDQKSTIVYPNKPRSGGM
LFHIFCFLFCTGEIRQEIPNADKSLEMEILKQKYR
LKDLTFLLEKQHELIKLIIQKMEIISETEDDDSHC
SFQDRFKKEQMEQRNSRWNTVLRAVKAKTHHLEP.

In an embodiment of the method, the TRPA1 polypeptide has at least 85% identity to a TRPA1 polypeptide having the following sequence:

(SEQ ID NO: 6)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQQ
PYGLKNLRPEFMQMQQIKELVMDEDNDGCTPLHYA
CRQGGPGSVNNLLGFNVSIHSKSKDKKSPLHFAAS
YGRINTCQRLLQDISDTRLLNEGDLHGMTPLHLAA
KNGHDKVVQLLLKKGALFLSDHNGWTALHHASMGG
YTQTMKVILDTNLKCTDRLDEDGNTALHFAAREGH
AKAVALLLSHNADIVLNKQQASFLHLALHNKRKEV
VLTIIRSKRWDECLKIFSHNSPGNKCPITEMIEYL
PECMKVLLDFCMLHSTEDKSCRDYYIEYNFKYLQC
PLEFTKKTPTQDVIYEPLTALNAMVQNNRIELLNH
PVCKEYLLMKWLAYGFRAHMMNLGSYCLGLIPMTI
LVVNIKPGMAFNSTGIINETSDHSEILDTTNSYLI
KTCMILVFLSSIFGYCKEAGQIFQQKRNYFMDISN
VLEWIIYTTGIIFVLPLFVEIPAHLOWQCGAIAVY
FYWMNFLLYLQRFENCGIFIVMLEVILKTLLRSTV
VFIFLLLAFGLSFYILLNLQDPFSSPLLSIIQTFS
MMLGDINYRESFLEPYLRNELAHPVLSFAQLVSFT
IFVPIVLMNLLIGLAVGDIAEVQKHASLKRIAMQV
ELHTSLEKKLPLWFLRKVDQKSTIVYPNKPRSGGM
LFHIFCFLFCTGEIRQEIPNADKSLEMEILKQKYR
LKDLTFLLEKQHELIKLIIQKMEIISETEDDDSHC
SFQDRFKKEQMEQRFCYENE.

In an embodiment of the method, the TRPA1 polypeptide has at least 85% identity to a TRPA1 polypeptide having the following sequence:

(SEQ ID NO: 7)
MKRSLRKMWRPGEKKEPQGVVYEDVPDDTEDFKES
LKVVFEGSAYGLQNFNKQKKLKRCDDMDTFFDYGN
TPLHCAVEKNQIESVKFLLSRGANPNLRNFNMMAP
LHIAVQGMNNEVMKVLLEHRTIDVNLEGENGNTAV
IIACTTNNSEALQILLKKGAKPCKSNKWGCFPIHQ
AAFSGSKECMEIILRFGEEHGYSRQLHINFMNNGK
ATPLHLAVQNGDLEMIKMCLDNGAQIDPVEKGRCT
AIHFAATQGATEIVKLMISSYSGSVDIVNTTDGCH
ETMLHRASLFDHHELADYLISVGADINKIDSEGRS
PLILATASASWNIVNLLLSKGAQVDIKDNFGRNFL
HLTVQQPYGLKNLRPEFMQMQQIKELVMDEDNDGC
TPLHYACRQGGPGSVNNLLGFNVSIHSKSKDKKSP
LHFAASYGRINTCQRLLQDISDTRLLNEGDLHGMT
PLHLAAKNGHDKVVQLLLKKGALFLSDHNGWTALH
HASMGGYTQTMKVILDTNLKCTDRLDEDGNTALHF
AAREGHAKAVALLLSHNADIVLNKQQASFLHLALH
NKRKEVVLTIIRSKRWDECLKIFSHNSPGNKCPIT
EMIEYLPECMKVLLDFCMLHSTEDKSCRDYYIEYN
FKYLQCPLEFTKKTPTQDVIYEPLTALNAMVQNNR
IELLNHPVCKEYLLMKWLAYGFRAHMMNLGSYCLG
LIPMTILVVNIKPGMAFNSTGIINETSDHSEILDT
TNSYLIKTCMILVFLSSIFGYCKEAGQIFQQKRNY
FMDISNVLEWIIYTTGIIFVLPLFVEIPAHLQWQC
GAIAVYFYWMNFLLYLQRFENCGIFIVMLEVILKT
LLRSTVVFIFLLLAFGLSFYILLNLQDPFSSPLLS
IIQTFSMMLGDINYRESFLEPYLRNELAHPVLSFA
QLVSFTIFVPIVLMNLLIGLAVGDIAEVQKHASLK
RIAMQVELHTSLEKKLPLWFLRKVDQKSTIVYPNK
PRSGGMLFHIFCFLFCTGEIRQEIPNADKSLEMEI
LKQKYRLKDLTFLLEKQHELIKLIIQKMEIISETE
DDDSHCSFQDRFKKEQMEQRNSRWNTVLRAVKAKT
HHLEPFCYENE.

In an embodiment of the method, the TRPA1 polypeptide comprises a sequence selected from the group consisting of SEQ ID NO: 1-7. In an embodiment of the method, the cell expresses a functional fragment of the TRPA1 polypeptide. In an embodiment, the fragment comprises at least about 20 amino acids from the N-terminus of the TRPA1 polypeptide. In an embodiment of the method, the expressed TRPA1 polypeptide or fragment thereof is a heterologous polypeptide.

In another aspect, a method of inducing cation influx in a cell is provided, in which the method comprises expressing a heterologous TRPA1 polypeptide or fragment thereof in a cell, and applying ultrasound to the cell, thereby inducing cation influx in the cell. In an embodiment of the method, the TRPA1 polypeptide comprises an amino acid sequence selected from the group consisting of SEQ ID NOs: 1-7, or a functional fragment thereof, e.g., an N-terminal fragment or portion comprising at least about 10-20 amino acids, or at least about 15-20 amino acids, or at least about 20 amino acids.

In an embodiment of the above-delineated aspects and embodiments, the cell is a mammalian cell, for example, a human cell. In an embodiment, of the above-delineated aspects and embodiments, the cell is a bacterial cell. In an embodiment of the above-delineated aspects and embodiments, the TRPA1 polypeptide is a human polypeptide. In embodiments of the methods, the cell is muscle cell, cardiac muscle cell, neuron, motor neuron, sensory neuron, interneuron, or insulin secreting cell. In an embodiment of the above-delineated aspects and embodiments, the ultrasound frequency is about 0.8 MHz to about 4 MHz. In an embodiment of the above-delineated aspects and embodiments, the ultrasound frequency is about 6.91 MHz. In an embodiment of the above-delineated aspects and embodiments, the ultrasound comprises an ultrasonic wave comprising a focal zone of about 1 cubic millimeter to about 1 cubic centimeter. In an embodiment of the above-delineated aspects and embodiments, the method further comprises contacting the cell with a microbubble prior to applying ultrasound. In an embodiment of the above-delineated aspects and embodiments, the cell is in vitro, in vivo, ex vivo, or in situ.

In another aspect, a method of treating a disease or disorder in a subject in need thereof is provided, in which the method involves (i) expressing in a cell of the subject a heterologous nucleic acid molecule encoding a TRPA1 polypeptide or fragment thereof; and (ii) applying ultrasound to the cell, thereby treating the disease or disorder in the subject. In an embodiment of the method, the disease or disorder is a neurological disease or disorder. In embodiments, the neurological disease or disorder is selected from the group consisting of Parkinson Disease, depression, obsessive-compulsive disorder, chronic pain, epilepsy or cervical spinal cord injury. In an embodiment of the method, the disease or disorder is muscle weakness. In an embodiment of the method, the subject is a mammalian subject. In an embodiment of the method, the subject is a human subject. In an embodiment of the method, the expressed heterologous TRPA1 polypeptide comprises a sequence selected from the group consisting of SEQ ID NOS: 1-7. In an embodiment of the method, the expressed heterologous TRPA1 polypeptide comprises a sequence selected from the group consisting of SEQ ID NOS: 4-7. An embodiment embraces a functional fragment of the polypeptide of SEQ ID NOS: 1-7, e.g., an N-terminal fragment or portion comprising at least about 10-20 amino acids, or at least about 15-20 amino acids, or at least about 20 amino acids.

In an embodiment of the above-delineated methods and embodiments thereof, the expressed TRPA1 polypeptide confers ultrasound sensitivity to the cell upon application of ultrasound. In an embodiment of the above-delineated methods and embodiments thereof, the ultrasound stimulates or triggers a response by the TRPA1-expressing cell. In an embodiment of the above-delineated methods and embodiments thereof, the cellular response comprises an influx of calcium ions into the cell.

In another aspect, a non-naturally occurring TRPA1 polypeptide comprising the amino acid sequence of SEQ ID NO: 4 is provided. An embodiment embraces a functional fragment of the polypeptide of SEQ ID NO: 4, e.g., an N-terminal fragment or portion comprising at least about 10-20 amino acids, or at least about 15-20 amino acids, or at least about 20 amino acids.

In another aspect, a non-naturally occurring TRPA1 polypeptide comprising the amino acid sequence of SEQ ID NO: 5 is provided. An embodiment embraces a functional fragment of the polypeptide of SEQ ID NO: 5, e.g., an N-terminal fragment or portion comprising at least about 10-20 amino acids, or at least about 15-20 amino acids, or at least about 20 amino acids.

In another aspect, a non-naturally occurring TRPA1 polypeptide comprising the amino acid sequence of SEQ ID NO: 6 is provided. An embodiment embraces a functional fragment of the polypeptide of SEQ ID NO: 6, e.g., an N-terminal fragment or portion comprising at least about 10-20 amino acids, or at least about 15-20 amino acids, or at least about 20 amino acids.

In another aspect, a non-naturally occurring TRPA1 polypeptide comprising the amino acid sequence of SEQ ID NO: 7 is provided. An embodiment embraces a functional fragment of the polypeptide of SEQ ID NO: 7, e.g., an N-terminal fragment or portion comprising at least about 10-20 amino acids, or at least about 15-20 amino acids, or at least about 20 amino acids.

In an aspect, a viral vector comprising a polynucleotide encoding a TRPA1 polypeptide or a functional fragment thereof is provided. In an embodiment, the TRPA1 polypeptide or the functional fragment thereof comprises an amino acid sequence selected from the group consisting of SEQ ID NOs: 1-7, or a functional fragment thereof. In an embodiment, the TRPA1 polypeptide or the functional fragment thereof comprises an amino acid sequence selected from the group consisting of SEQ ID NOs: 4-7, or a functional fragment thereof, e.g., an N-terminal fragment or portion comprising at least about 10-20 amino acids, or at least about 15-20 amino acids, or at least about 20 amino acids. In an embodiment, the vector is a lentiviral vector or an adeno-associated viral vector.

In an aspect, a cell comprising the TRPA1 polypeptide of any of the above-delineated aspects and/or embodiments thereof is provided.

In an aspect, a cell comprising the viral vector of any of the above-delineated aspects and/or embodiments thereof is provided.

In an aspect, a composition comprising the TRPA1 polypeptide of any of the above-delineated aspects and/or embodiments thereof is provided.

In an aspect, a composition comprising the viral vector of any of the above-delineated aspects and/or embodiments thereof is provided.

In an aspect, a composition comprising the cell of any of the above-delineated aspects and/or embodiments thereof is provided.

In an embodiment of any of the above-delineated aspects of the composition and/or embodiments thereof, the composition further comprises a pharmaceutically acceptable carrier, excipient, or diluent.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of the aspects and embodiments described herein. The following definitions are provided to facilitate understanding of certain terms used frequently herein and are not meant to limit the scope of the present disclosure.

By “TRPA1 polypeptide” is meant a human transient receptor potential cation channel or fragment thereof capable of conferring ultrasound sensitivity on a neuron and having at least about 85% amino acid sequence identity to NCBI Ref. Seq. NP_015628.2, XP_016869435.1, XP_011515927.1, XP_011515926.1, GenBank: EAW869861, or a human ortholog thereof. In embodiments, the TRPA1 polypeptide has at least 88%, at least 90%, at least 91%, at least 92%, at least 93%, at least 94%, at least 95%, at least 96%, at least 97%, at least 98%, at least 99%, or 100% amino acid sequence identity to the above-noted sequences. An exemplary sequence of TRPA1 is NCBI Reference Sequence: XP_016869435.1, which is reproduced below:

```
(SEQ ID NO: 1)
      1 MKRSLRKMWR PGEKKEPQGV VYEDVPDDTE DFKESLKVVF EGSAYGLQNF NKQKKLKRCD
    61 DMDTFFLHYA AAEGQIELME KITRDSSLEV LHEMDDYGNT PLHCAVEKNQ IESVKFLLSR
  121 GANPNLRNEN MMAPLHIAVQ GMNNEVMKVL LEHRTIDVNL EGENGNTAVI IACTTNNSEA
  181 LQILLKKGAK PCKSNKWGCF PIHQAAFSGS KECMEIILRF GEEHGYSRQL HINFMNNGKA
  241 TPLHLAVQNG DLEMIKMCLD NGAQIDPVEK GRCTAIHFAA TQGATEIVKL MISSYSGSVD
  301 IVNTTDGCHE TMLHRASLFD HHELADYLIS VGADINKIDS EGRSPLILAT ASASWNIVNL
  361 LLSKGAQVDI KDNFGRNFLH LTVQQPYGLK NLRPEFMQMQ QIKELVMDED NDGCTPLHYA
  421 CRQGGPGSVN NLLGFNVSIH SKSKDKKSPL HFAASYGRIN TCQRLLQDIS DTRLLNEGDL
  481 HGMTPLHLAA KNGHDKVVQL LLKKGALFLS DHNGWTALHH ASMGGYTQTM KVILDTNLKC
  541 TDRLDEDGNT ALHFAAREGH AKAVALLLSH NADIVLNKQQ ASFLHLALHN KRKEVVLTII
  601 RSKRWDECLK IFSHNSPGNK CPITEMIEYL PECMKVLLDF CMLHSTEDKS CRDYYIEYNF
  661 KYLQCPLEFT KKTPTQDVIY EPLTALNAMV QNNRIELLNH PVCKEYLLMK WLAYGFRAHM
  721 MNLGSYCLGL IPMTILVVNI KPGMAFNSTG IINETSDHSE ILDTTNSYLI KTCMILVFLS
  781 SIFGYCKEAG QIFQQKRNYF MDISNVLEWI IYTTGIIFVL PLFVEIPAHL QWQCGAIAVY
  841 FYWMNFLLYL QRFENCGIFI VMLEVILKTL LRSTVVFIFL LLAFGLSFYI LLNLQDPFSS
  901 PLLSIIQTFS MMLGDINYRE SFLEPYLRNE LAHPVLSFAQ LVSFTIFVPI VLMNLLIGLA
  961 VGDIAEVQKH ASLKRIAMQV ELHTSLEKKL PLWFLRKVDQ KSTIVYPNKP RSGGMLFHIF
1021 CFLFCTGEIR QEIPNADKSL EMEILKQKYR LKDLTFLLEK QHELIKLIIQ KMEIISETED
1081 DDSHCSFQDR FKKEQMEQRN SRWNTVLRAV KAKTHHLEP.
```

An exemplary sequence of NP 015628.2 follows:

| (SEQ ID NO: 2) | |
|---|---|
| 1 | MKRSLRKMWR PGEKKEPQGV VYEDVPDDTE DFKESLKVVF EGSAYGLQNF NKQKKLKRCD |
| 61 | DMDTFFLHYA AAEGQIELME KITRDSSLEV LHEMDDYGNT PLHCAVEKNQ IESVKFLLSR |
| 121 | GANPNLRNFN MMAPLHIAVQ GMNNEVMKVL LEHRTIDVNL EGENGNTAVI IACTTNNSEA |
| 181 | LQILLKKGAK PCKSNKWGCF PIHQAAFSGS KECMEIILRF GEEHGYSRQL HINFMNNGKA |
| 241 | TPLHLAVQNG DLEMIKMCLD NGAQIDPVEK GRCTAIHFAA TQGATEIVKL MISSYSGSVD |
| 301 | IVNTTDGCHE TMLHRASLFD HHELADYLIS VGADINKIDS EGRSPLILAT ASASWNIVNL |
| 361 | LLSKGAQVDI KDNFGRNFLH LTVQQPYGLK NLRPEFMQMQ QIKELVMDED NDGCTPLHYA |
| 421 | CRQGGPGSVN NLLGFNVSIH SKSKDKKSPL HFAASYGRIN TCQRLLQDIS DTRLLNEGDL |
| 481 | HGMTPLHLAA KNGHDKVVQL LLKKGALFLS DHNGWTALHH ASMGGYTQTM KVILDTNLKC |
| 541 | TDRLDEDGNT ALHFAAREGH AKAVALLLSH NADIVLNKQQ ASFLHLALHN KRKEVVLTII |
| 601 | RSKRWDECLK IFSHNSPGNK CPITEMIEYL PECMKVLLDF CMLHSTEDKS CRDYYIEYNF |
| 661 | KYLQCPLEFT KKTPTQDVIY EPLTALNAMV QNNRIELLNH PVCKEYLLMK WLAYGFRAHM |
| 721 | MNLGSYCLGL IPMTILVVNI KPGMAFNSTG IINETSDHSE ILDTTNSYLI KTCMILVFLS |
| 781 | SIFGYCKEAG QIFQQKRNYF MDISNVLEWI IYTTGIIFVL PLFVEIPAHL QWQCGAIAVY |
| 841 | FYWMNFLLYL QRFENCGIFI VMLEVILKTL LRSTVVFIFL LLAFGLSFYI LLNLQDPFSS |
| 901 | PLLSIIQTFS MMLGDINYRE SFLEPYLRNE LAHPVLSFAQ LVSFTIFVPI VLMNLLIGLA |
| 961 | VGDIAEVQKH ASLKRIAMQV ELHTSLEKKL PLWFLRKVDQ KSTIVYPNKP RSGGMLFHIF |
| 1021 | CFLFCTGEIR QEIPNADKSL EMEILKQKYR LKDLTFLLEK QHELIKLIIQ KMEIISETED |
| 1081 | DDSHCSFQDR FKKEQMEQRN SRWNTVLRAV KAKTHHLEP. |

In some embodiments, a TRPA1 polypeptide comprises a fragment of NCBI Reference Sequence: XP_016869435.1. In some embodiments, the TRPA1 fragment comprises at least about 10, 15, 20, 30, 40, 50, 60 or more amino acids from the N-terminal region of TRPA1. In other embodiments, the TRPA1 fragment comprises a cytoplasmic ankyrin portion of the TRPA1 polypeptide.

For specific proteins described herein (e.g., TRPA1 or hsTRPA1), the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

By “TRPA1 polynucleotide” is meant a nucleic acid molecule encoding a TRPA1 polypeptide. In particular embodiments, the codons of the TRPA1 polynucleotide are optimized for expression in an organism of interest (e.g., optimized for human expression, bacterial expression, murine expression). The sequence of an exemplary TRPA1 polynucleotide is provided at NCBI Ref. Seq.: NM_007332.3, which is reproduced herein below:

| (SEQ ID NO: 3) | |
|---|---|
| 1 | ccagaagttc tccagggctt ccgcagagcg actttttcgc tgcctgtgag ctgcagcgcg |
| 61 | ggagagctcg ggctcgcgcg gaccccagcg cctggcaggc tgacagcgct ctctcgcccc |
| 121 | aggtgcccgc gegcgtggtg agcagctgca ccaggtggcg tccggggtgg ggtcaatgaa |
| 181 | gegcagcctg aggaagatgt ggcgccctgg agaaaagaag gagccccagg gcgttgtcta |
| 241 | tgaggatgtg ccggacgaca cggaggattt caaggaatcg cttaaggtgg tttttgaagg |
| 301 | aagtgcatat ggattacaaa actttaataa gcaaaagaaa ttaaaaagat gtgacgatat |
| 361 | ggacaccttc ttcttgcatt atgctgcagc agaaggccaa attgagctaa tggagaagat |
| 421 | caccagagat tcctctttgg aagtgctgca tgaaatggat gattatggaa atacccctct |
| 481 | gcattgtgct gtagaaaaaa accaaattga aagcgttaag tttcttctca gcagaggagc |
| 541 | aaacccaaat ctccgaaact tcaacatgat ggctcctctc cacatagctg tgcagggcat |
| 601 | gaataatgag gtgatgaagg tcttgcttga gcatagaact attgatgtta atttggaagg |
| 661 | agaaaatgga aacacagctg tgatcattgc gtgcaccaca aataatagcg aagcattgca |
| 721 | gattttgctt aaaaaaggag ctaagccatg taaatcaaat aaatggggat gtttccctat |
| 781 | tcaccaagct gcattttcag gttccaaaga atgcatggaa ataatactaa ggtttggtga |
| 841 | agagcatggg tacagtagac agttgcacat taactttatg aataatggga aagccacccc |
| 901 | tctccacctg gctgtgcaaa atggtgactt ggaaatgatc aaaatgtgcc tggacaatgg |
| 961 | tgcacaaata gacccagtgg agaagggaag gtgcacagcc attcattttg ctgccaccca |
| 1021 | gggagccact gagattgtta aactgatgat atcgtcctat tctggtagcg tggatattgt |
| 1081 | taacacaacc gatggatgtc atgagaccat gcttcacaga gcttcattgt ttgatcacca |
| 1141 | tgagctagca gactatttaa tttcagtggg agcagatatt aataagatcg attctgaagg |
| 1201 | acgctctcca cttatattag caactgcttc tgcatcttgg aatattgtaa atttgctact |
| 1261 | ctctaaaggt gcccaagtag acataaaaga taattttgga cgtaattttc tgcatttaac |
| 1321 | tgtacagcaa ccttatggat taaaaaatct gcgacctgaa tttatgcaga tccaacagat |
| 1381 | caaagagctg gtaatggatg aagacaacga tgggtgtact cctctacatt atgcatgtag |
| 1441 | acaggggggc cctggttctg taaataacct acttggcttt aatgtgtcca ttcattccaa |
| 1501 | aagcaaagat aagaaatcac ctctgcattt tgcagccagt tatgggcgta tcaatacctg |
| 1561 | tcagaggctc ctacaagaca taagtgatac gaggcttctg aatgaaggtg accttcatgg |
| 1621 | aatgactcct ctccatctgg cagcaaagaa tggacatgat aaagtagttc agcttcttct |
| 1681 | gaaaaaaggt gcattgtttc tcagtgacca caatggctgg acagctttgc atcatgcgtc |
| 1741 | catgggcggg tacactcaga ccatgaaggt cattcttgat actaatttga agtgcacaga |
| 1801 | tcgcctggat gaagacggga acactgcact tcactttgct gcaagggaag gccacgccaa |
| 1861 | agccgttgcg cttcttctga gccacaatgc tgacatagtc ctgaacaagc agcaggcctc |
| 1921 | ctttttgcac cttgcacttc acaataagag gaaggaggtt gttcttacga tcatcaggag |
| 1981 | caaaagatgg gatgaatgtc ttaagatttt cagtcataat tctccaggca ataaatgtcc |
| 2041 | aattacagaa atgatagaat acctccctga atgcatgaag gtacttttag atttctgcat |
| 2101 | gttgcattcc acagaagaca agtcctgccg agactattat atcgagtata atttcaaata |
| 2161 | tcttcaatgt ccattagaat tcaccaaaaa aacacctaca caggatgtta tatatgaacc |
| 2221 | gattacagcc ctcaacgcaa tggtacaaaa taaccgcata gagcttctca atcatcctgt |
| 2281 | gtgtaaagaa tatttactca tgaaatggtt ggcttatgga tttagagctc atatgatgaa |
| 2341 | tttaggatct tactgtcttg gtctcatacc tatgaccatt ctcgttgtca atataaaacc |
| 2401 | aggaatggct ttcaactcaa ctggcatcat caatgaaact agtgatcatt cagaaatact |
| 2461 | agataccacg aattcatatc taataaaaac ttgtatgatt ttagtgtttt tatcaagtat |
| 2521 | atttgggtat tccaaagaag cggggcaaat tttccaacag aaaaggaatt attttatgga |
| 2581 | tataagcaat gttcttgaat ggattatcta cacgacgggc atcatttttg tgctgccctt |
| 2641 | gtttgttgaa ataccagctc atctgcagtg gcaatgtgga gcaattgctg tttacttcta |
| 2701 | ttggatgaat ttcttattgt atcttcaaag atttgaaaat tgtggaattt ttattgttat |
| 2761 | gttggaggta attttgaaaa ctttgttgag gtctacagtt gtatttatct tccttcttct |
| 2821 | ggcttttgga ctcagctttt acatcctcct gaatttacag gatcccttca gctctccatt |
| 2881 | gctttctata atccagacct tcagcatgat gctaggagat atcaattatc gagagtcctt |
| 2941 | cctagaacca tatctgagaa atgaattggc acatccagtt ctgtcctttg cacaacttgt |
| 3001 | ttccttcaca atatttgtcc caattgtcct catgaattta cttattggtt tggcagttgg |
| 3061 | cgacattgct gaggtccaga aacatgcatc attgaagagg atagctatgc aggtggaact |
| 3121 | tcataccagc ttagagaaga agctgccact ttggtttcta cgcaaagtgg atcagaaatc |
| 3181 | caccatcgtg tatcccaaca aacccagatc tggtgggatg ttattccata tattctgttt |
| 3241 | tttattttgc actggggaaa taagacaaga aataccaaat gctgataaat ctttagaaat |
| 3301 | ggaaatatta aagcagaaat accggctgaa ggatcttact tttctcctgg aaaaacagca |
| 3361 | tgagctcatt aaactgatca ttcagaagat ggagatcatc tctgagacag aggatgatga |
| 3421 | tagccattgt tcttttcaag acaggtttaa gaaagagcag atggaacaaa ggaatagcag |
| 3481 | atggaatact gtgttgagag cagtcaaggc aaaaacacac catcttgagc cttagctcct |
| 3541 | cagaccttca gtgaggcttc taatgggggg tgcatgactt getggttcta actttcaatt |
| 3601 | taaaaagagt gaggaagaag cagaatgatt cattttgctg cgtgtgaaat catggttcct |
| 3661 | gcatgctgta taaaagtaaa ccatctttta tectctattc atattttcta ccaatcacta |
| 3721 | tgtattgggg atatctttgc agatatgttc aaattggact ggactttgat gagatataat |
| 3781 | ctcattattt gaatgggtag aaaatgaatt tgctagaaca cacattttta atgaaaagaa |
| 3841 | gtaataaatg taactattaa gctaaaatgc aaatgtcagt actgaattcc tgcttgttaa |
| 3901 | ttacataata tgtgatgctc tagaaaatag tcacaagtat taataatgcc ttagatgata |
| 3961 | gtcttaaata ttaggttgag gtctacctaa cctaagctgc ttcctggaaa gcttcatgtt |
| 4021 | gaaagaacct atgggtggca ccatgtggac ttttctgtcc ctactgtgat gaatagcccc |
| 4081 | acccttcttg ctgtccccaa cacacctgat gtcactttga gccatatagt tgaagtacaa |
| 4141 | attaataggc cttatgatat gcacgaattt tactatagat aatatatgtt gtttctggtt |
| 4201 | ttgtttgcca atgagcataa taaatgtaaa acctatatag tatccctgtg attattgtat |
| 4261 | gagcctttgt ttgagatttg aaaacaacat ggctccatca catattccct tttttctttt |
| 4321 | gatgtctact caaatcatga attaatcaca tacctcatca ttaatctttt caaggtcctt |
| 4381 | ctattgtttt gtctgatttt ctccatcatc ctgattagca tctttattcc ctcactaccc |
| 4441 | ccaggagata ttcactgtaa tgaatatgtc tttggctatg tatgtgtcct tgtgttatgt |
| 4501 | tgtacagtgt tcttttgagt ctgttattat ttacacagat gttattatgc tatagcttct |
| 4561 | atttctgttt ttgcttctta tttctcttat aattctcact tatttcctat tttttctact |
| 4621 | catttctatt tgttactcct ttttactgga catgatgttt acaagataca actgtgttac |
| 4681 | tgtattccat ctagtacggg gcctttggtg tggcttacta tttcattgtg tgcacccacc |
| 4741 | cacccaccac actggacttt tctagagatg gacagcttgg ttacctccac cttcctgcac |
| 4801 | tcattctcaa acatactgat gttcatacaa accagcagag tgctgaggga cgatatgtac |
| 4861 | tattacaaaa ccagacactt ttacattcat ggtccaacag atcacatggc ctagaggcaa |
| 4921 | tcttgcatat accttaatct ttgatatgaa taatatcttt gttctttata tttcttaaaa |
| 4981 | cagaaagggt ggaaaatcac tatacagaag caatatccaa agatctcctg atcataaaga |
| 5041 | caaggggtct tttcagtctt ccctctcctc aaaccttgtg tagcattgca caatatagat |
| 5101 | ctcagtcaac attcactgag tcccaagaat gtgagaaaca ctgtaccatg cctgtcatgc |
| 5161 | gaaatattta aataaacaga ttgtcttaca a. |

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide is implicit in each described sequence with respect to the expression product, but not with respect to actual probe sequences.

By “altered” is meant an increase or decrease. An increase is any positive change, e.g., by at least about 5%, 10%, or 20%; preferably by about 25%, 50%, 75%, or even by 100%, 200%, 300% or more. A decrease is a negative change, e.g., a decrease by about 5%, 10%, or 20%; preferably by about 25%, 50%, 75%; or even an increase by 100%, 200%, 300% or more.

The terms “comprises”, “comprising”, and are intended to have the broad meaning ascribed to them in U.S. Patent Law and can mean “includes”, “including” and the like.

“Contacting” is used in accordance with its plain ordinary meaning and refers to the process of allowing at least two distinct species (e.g. chemical compounds including biomolecules, or cells) to become sufficiently proximal to react, interact, affect or physically touch. It should be appreciated, however, that the resulting reaction product can be produced directly from a reaction between the added reagents or from an intermediate from one or more of the added reagents, which can be produced in the reaction mixture. Contacting may include allowing two species to react, interact, or physically touch, wherein the two species may be a recombinant viral particle as described herein and a cell. In embodiments, the two species are an ultrasound contrast agent that is exposed to ultrasound and a cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

By “mammal” is meant any warm-blooded animal including but not limited to a human, cow, horse, pig, sheep, goat, bird, mouse, rat, dog, cat, monkey, baboon, or the like. Preferably, the mammal is a human.

“Nucleic acid” refers to deoxyribonucleotides or ribonucleotides and polymers thereof in either single- or double-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and 2-O-methyl ribonucleotides.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

By “positioned for expression” is meant that a polynucleotide (e.g., a DNA molecule) is positioned adjacent to a DNA sequence, which directs transcription, and, for proteins, translation of the sequence (i.e., facilitates the production of, for example, a recombinant polypeptide of the aspects and embodiments described herein, or an RNA molecule).

The term “plasmid” or “vector” refers to a nucleic acid molecule that encodes for genes and/or regulatory elements necessary for the expression of genes. Expression of a gene from a plasmid or vector can occur in cis or in trans. If a gene is expressed in cis, the gene and the regulatory elements are encoded by the same plasmid and vector. Expression in trans refers to the instance where the gene and the regulatory elements are encoded by separate plasmids or vectors.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” or “control” is meant a standard condition. For example, an untreated cell, tissue, or organ that is used as a reference. In some embodiments, a cell over-expressing a recombinant TRPA1 polypeptide is compared to a cell that is not expressing any TRPA1 or that is not expressing recombinant TRPA1 (i.e., a cell that is only expressing endogenous TRPA1).

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods. The term heterologous may be used interchangeably with the terms exogenous, non-native, non-naturally occurring, or recombinant herein.

The term “subject” as used herein refers to a vertebrate, preferably a mammal (e.g., dog, cat, rodent, horse, bovine, rabbit, goat, or human). In particular embodiments, a subject is a human subject or a patient.

By “transformed cell” is meant a cell into which (or into an ancestor of which) has been introduced, by means of recombinant DNA techniques, a polynucleotide molecule encoding (as used herein) a polypeptide of the described aspects and embodiments.

As used herein, the terms “treat,” treating,” “treatment,” and the like refer to reducing, abating, decreasing, diminishing, allaying, alleviating, or ameliorating a disorder and/or symptoms associated therewith. It will be appreciated that, although not precluded, treating a disorder or condition does not require that the disorder, condition or symptoms associated therewith be completely eliminated.

The terms “transfection”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein (e.g. Tat, Rev) relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, monkeys, goat, sheep, cows, deer, and other non-mammalian animals. In embodiments, a patient is human.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1I. hsTRPA1 confers sensitivity to single, short duration ultrasound pulses in HEK cells. FIG. 1A: Schematic showing the 6.91 MHz lithium niobate transducer delivering ultrasound stimuli to cells. Plot showing FIG. 1B: The percent of transfected versus percent of transfected cells that were activated cells after ultrasound stimulation for 191 cDNAs and FIG. 1C: the top responders and their homologs compared to reported ultrasound-sensitive candidates. FIG. 1D: Representative image showing hsTRPA1 expression co-localized with membrane-targeted EGFP-CAAX membrane marker in HEK 293 cells. FIG. 1E: GCaMP6f signal in HEK cells expressing hsTRPA1 before and after ultrasound stimulation. ROIs identify transfected cells (dTomato positive, dTom+). Scale bar 20 μm. GCaMP6f peak amplitude in hsTRPA1- or dTom-expressing (control) HEK cells stimulated with ultrasound. FIG. 1F: TRPA1 agonist (NMM, 100 μM), ultrasound alone or TRPA1 antagonist (HC-030031, 40 μM). FIG. 1G: Schematic showing the cell-attached configuration for electrophysiology with a DIC image of a representative HEK cell. FIG. 1H: Representative gap-free voltage-clamp trace of dTom control- or hsTRPA1-expressing HEK to 100 ms, 0.15 MPa ultrasound stimuli. FIG. 1I: Mean peak amplitude (pA) from HEK cells expressing hsTRPA1 alone, and hsTRPA1 treated with vehicle or TRPA1 antagonist (HC-030301 40 μM). Numbers of cells analyzed is indicated above each bar. FIG. 1C: **p<0.01, ***p<0.001, ****p<0.0001 by logistic regression, FIGS. 1F, 1I: **p<0.01, ****p<0.0001 by Mann-Whitney test.

FIGS. 2A-2J. The N-terminal region of hsTRPA1, actin cytoskeleton and cholesterol contribute to ultrasound sensitivity. FIG. 2A: Mammalian and non-mammalian alignments of the TRPA1 N-terminal tip region (amino acids (aa) 1-25) from homologs tested for ultrasound sensitivity. GCaMP6f peak amplitude upon FIG. 2B. FIG. 2B: Ultrasound stimulation or FIG. 2C. FIG. 2C: Treatment with AITC (33 μM) in HEK cells transfected with either full-length hsTRPA1 or channels containing deletions of the whole N-terminal tip (Δ(1-61)), an initial subsection of the N-tip (Δ(1-25)) or only ankyrin repeat 1 (ΔANK1) without altering the pore or transmembrane regions. GCaMP6f peak amplitude upon FIG. 2D. FIG. 2D: Ultrasound stimulation or treatment with AITC (33 μM), (FIG. 2E), in HEK cells transfected with either full-length hsTRPA1 or chimeras in which the N-tip from alligator TRPA1 (N-tip a.m) or from zebrafish (N-tip d.r) was swapped in FIG. 2F. FIG. 2F: GCaMP6f peak amplitude following ultrasound stimulation in cells expressing hsTRPA1 after treatment with agents that either stabilize (green) or destabilize (red) microtubules and actin filaments compared to vehicle control. FIG. 2G: Transmembrane 2 domain sequence alignment across species tested for ultrasound sensitivity with Cholesterol Recognition/interaction Amino acid Consensus (CRAC) domain outlined. FIG. 2H: GCaMP6f peak amplitude in hsTRPA1-expressing HEK cells upon ultrasound stimulation or AITC treatment (33 μM) after incubation with MCD (5 mM) or control. FIG. 2I: GCaMP6f peak amplitude in HEK cells expressing either WT hsTRPA1 or a mutant with TM2 CRAC domain disrupted (Y785S) upon ultrasound stimulation or FIG. 2J. FIG. 2J: AITC treatment (33 μM); Numbers on each bar indicate numbers of cell analyzed. p>0.05, **p<0.01, ***p<0.001, ****p<0.0001 by Kruskal-Wallis rank test and Dunn's test for multiple comparisons.

FIGS. 3A-3M. hsTRPA1 potentiates calcium responses and evoked action potentials upon ultrasound stimulation in rodent primary neurons in vitro. FIG. 3A: Representative images showing mouse primary neurons day in vitro (DIV) 12, expressing hsTRPA1 or controls. FIG. 3B: GCaMP6f fluorescence in hsTRPA1 expressing neurons before and after ultrasound stimulus. Plots showing peak amplitude of GCaMP6f fluorescence upon FIG. 3C. FIG. 3C: 2.5 MPa ultrasound stimuli of 100 ms duration, and FIG. 3D. FIG. 3D: 100 ms stimuli at different pressures. FIG. 3E: Average ratio of change in fluorescence to baseline fluorescence in neurons expressing hsTRPA1 or control plasmids during repetitive 100 ms, 2.5 MPa ultrasound stimulation. The number of GCaMP6f-expressing neurons analyzed is indicated above each bar. FIG. 3C: ****p<0.0001, by Mann-Whitney U test; FIG. 3D: *p<0.05, **p<0.01, ****p<0.0001 by two-way ANOVA with Geisser-Greenhouse correction. FIG. 3F: Schematic showing whole cell patch electrophysiology of neurons expressing hsTRPA1 used for both voltage-clamp and current-clamp recordings. Representative gap-free voltage-clamp traces of control neurons (FIG. 3G) or neurons expressing hsTRPA1 (FIG. 3H) upon ultrasound stimuli in the 0.25 MPa range. FIG. 3I: Plot showing peak amplitude response to ultrasound stimuli in neurons expressing hsTRPA1 or controls (Cre). 6.91 MHz 0.25 MPa ultrasound in DIV 11-14 rat primary neurons under current-clamp mode elicits subthreshold voltage changes in controls (FIG. 3J) and action potentials (FIG. 3K) in TRPA1 expressing cells. FIG. 3L: Percent of trials in which an action potential was elicited by ultrasound in controls and TRPA1-expressing neurons. FIG. 3M: Results showing that resting membrane potential is not altered in primary neurons upon expression of TRPA1. n.s. p>0.05, ***p<0.001, ****p<0.0001 by unpaired, two-tailed Mann-Whitney U test.

FIGS. 4A-4K. hsTRPA1 enables sonogenetic activation of mouse layer V motor cortex neurons in vivo. FIG. 4A: Schematic showing expression of hsTRPA1 or GFP controls in the left motor cortex of Npr3-Cre transgenic mice innervating the right fore and hindlimbs allowing these to be controlled with ultrasound stimuli. FIG. 4B: Images showing expression of hsTRPA1 and GFP (co-injection marker) in layer 5 cortical neurons in vivo. FIG. 4C: Representative EMG responses to 10 ms and 100 ms ultrasound stimuli from animals expressing hsTRPA1 and controls. FIG. 4D Visible right limb movements were scored in response to 100 ms ultrasound pulses of varying intensities. Plots showing percent of right fore and hindlimb (FIG. 4E) and left fore and hindlimb (FIG. 4F) EMG responses relative to number of stimulations pooled across all intensities. Plots showing (FIG. 4G) latency between the start of the ultrasound pulse and subsequent EMG response, and (FIG. 4H) duration from the start of the EMG response until the signal returned to baseline. FIG. 4I: Percent of c-fos+ GFP+ neurons quantified from sections taken at −700 μM intervals throughout the GFP+ region of the cortex. Representative images showing co-localization of FIG. 4J, c-fos and GFP and FIG. 4K, c-fos and DAPI within GFP positive neurons. FIGS. 4D-F, n=5/group; FIGS. 4G and H, n=39-138; FIG. 4I, n=3-4/group *p<0.05, **p<0.01, ****p<0.0001 compared to GFP control by two-way ANOVA followed Tukey's multiple comparisons.

FIGS. 5A-5M: Characterization of TRPA1 calcium responses in HEK cells. FIG. 5A: Plot showing maximum temperature increases under different ultrasound stimulation parameters. n=3 assays/condition. FIG. 5B: Time series of ultrasound-evoked temperature changes in the cell culture dish during stimulation. FIG. 5C: % active hsTRPA1 and dTom transfected cells in response to ultrasound at different pressure and durations. n=3 coverslips/condition. FIG. 5D: Image showing dTom+ ROIs in HEK cells expressing hsTRPA1 and change in GCaMP fluorescence upon ultrasound stimulation (FIG. 5E) or FIG. 5F. FIG. 5F: application of NMM in individual cells. FIG. 5G: Image showing HEK cells expressing dTom control and change in GCaMP fluorescence upon FIG. 5H. FIG. 5H: Ultrasound stimulation in individual cells or FIG. 5I. FIG. 5I: Application of NMM in individual cells. FIG. 5J: HEK cells expressing hsTRPA1 respond to TRPA1 agonists, N-methyl maleimide (NMM, 100 μM). and allyl isothiocyanate (AITC 33 μM). n=3 coverslips/condition. FIG. 5K: HEK cells expressing mouse-Piezol respond to yoda-1(10 μM), but not GsMTx-4-4 or ultrasound. n=3 coverslips/condition. FIG. 5L: HEK cells expressing human-TRPV1 respond to capsaicin (3 μM), but not AITC, GsMTx-4-4 or ultrasound. n=3 coverslips/condition. Number of cells analyzed is shown on each bar. ****p<0.0001, by Kruskal-Wallis rank test and Dunn's test for multiple comparisons. Scale bar, 20 μm. FIG. 5M: Response to AITC in HEK cells expressing TRPA1 from tested species.

FIGS. 6A-6J: Ultrasound stimulation at 2.5 MPa is safe as assessed by intracellular uptake of propidium iodide. FIG. 6A: Image showing bright field (BF) image for GCaMP6f-HEK cells, and the corresponding GFP channel (FIG. 6B) and propidium iodide channel (FIG. 6C) before ultrasound stimulation. Multiple trials with ultrasound stimulation at 2.5 MPa 100 ms had no effect on the intracellular levels of propidium iodide (FIG. 6D) n=3 stims. FIG. 6E: Image showing HEK cells used for the positive control, including GFP channel (FIG. 6F) and propidium iodide channel before treatment (FIG. 6G). Addition of 0.1% Triton-X induced a significant increase of intracellular propidium iodide (FIG. 6H). Time course for the propidium iodide signal for an ultrasound stimulated cell highlighted in FIG. 6A, shown in FIG. 6I, and for a cell treated with propidium iodide highlighted in FIG. 6E, shown in FIG. 6J. Scale bar, 20 μm.

FIGS. 7A-7D: Electrophysiological properties of HEK cells expressing hsTRPA1. FIG. 7A: Representative traces in HEK cells expressing dTom only (control) or hsTRPA1 before ultrasound stimulation, showing increased spontaneous activity in hsTRPA1-expressing cells. FIG. 7B: I-V plot of HEK cells expressing dTom control or hsTRPA1. FIG. 7C: HEK cells expressing hsTRPA1 have more frequent ultrasound-triggered membrane events compared to dTom controls. FIG. 7D: Summary of relative peak amplitude responses (I/Imax) in HEK cells expressing dTom or hsTRPA1. Events were sampled from N=8 cells/group. ****p<0.0001 compared to control by unpaired, two-tailed t-test.

FIGS. 8A-8C: TRPA1 sequence alignment across homologs tested for ultrasound sensitivity. FIG. 8A: Schematic of TRPA1 showing the N-terminal region (green), 16 ankyrin repeats (orange) and the 6 transmembrane domains (pink). Mammalian and non-mammalian alignments of TRPA1 homologs tested for ultrasound sensitivity, depicting different domains and % identity compared to hsTRPA1 for the whole protein (FIG. 8B) and for Ankyrin 1 (FIG. 8C). % indicates % identity between 65% consensus sequence and hsTRPA1.

FIGS. 9A-9D: Expression of TRPA1 mutants in HEK293T cells. Immunohistochemistry showing expression and correct trafficking of myc-tagged TRPA1 constructs with CRAC Y785S mutation (FIG. 9A); FIG. 9B: N-terminal tip (aa 1-25) deletion; FIG. 9C: amTRPA1 N-terminal tip swapped into hsTRPA1; and FIG. 9D: drTRPA1 N-terminal tip swapped into hsTRPA1.

FIGS. 10A-10C: Cytoskeletal inhibitors alter hsTRPA1 cell morphology and function. FIG. 10A: HEK293 cells expressing hsTRPA1 have disrupted microtubules after treatment with nocodazole. FIG. 10B: actin filaments after cytochalasin-D treatment, but not vehicle controls. Microtubules are labeled using anti-alpha tubulin, while actin filaments are assessed by phalloidin staining. FIG. 10C: Treating HEK293 cells expressing hsTRPA1 with cytochalasin D or nocodazole has no significant effect on AITC responses compared to vehicle controls. In contrast, HEK293-hsTRPA responses to AITC were reduced after treatment latrunculin A and j asplakinolide and paclitaxel, presumably due to poor cell health. n=3 coverslips/condition. Numbers of cells analyzed are shown in each bar. **p<0.01, ****p<0.0001 Kruskal-Wallis rank test and Dunn's test for multiple comparisons.

FIGS. 11A-11F: hsTRPA1 RNA is not detected in the E18 or adult mouse cortex. Results from a Base Scope in situ hybridization experiment in adult dorsal root ganglia (DRG) and cortex taken from (FIGS. 11A, 11B) wild-type (WT) C57Bl6/J mouse, or (FIGS. 11C, 11D) TRPA1−/− mice, as well as E18 (FIG. 11E), DRG and (FIG. 11F) cortex taken from a WT C57Bl6 embryo. Positive signal is detected as magenta puncta within cell bodies and was only detected in the adult WT DRG, as expected.

FIGS. 12A-12F: Ultrasound-evoked responses in primary neurons are independent of TRPA1. FIG. 12A: Dose response curve of hsTRPA1-, and Cre-control expressing neurons to AITC. n=3 coverslips/condition. FIG. 12B: Image showing GCaMP fluorescence in primary neurons infected with control Cre virus before and after ultrasound stimulation. FIGS. 12C-12E: Representative traces and graphs showing magnitude of ultrasound-induced responses in representative control (Cre) or hsTRPA1-expressing neurons. Primary neurons from TRPA1 knockout mice (TRPA1−/−) responded to ultrasound (FIG. 12F). n=3 coverslips/condition. Number of cells analyzed is shown in each bar.

FIGS. 13A-13H: Characterizing ultrasound responses in hsTRPA1 expressing primary neurons. FIG. 13A: Distribution of ultrasound responses to 100 ms 2.5 MPa in control and TRPA1-myc primary neurons (FIG. 13B). FIG. 13C: Removing outliers reduces the maximum value observed for TRPA1-myc infected neurons but a statistically significant difference between controls and TRPA1 (pv<0.001) was observed; thus confirming the robustness of the effect. Plot showing time to 60% of peak response (latency), (FIG. 13D) and time between 63% rise and 63% decay (response width) (FIG. 13E) after ultrasound stimulation at 100 msecs and different peak negative pressures in hsTRPA1 expressing primary neurons. Plots showing distribution of latency (FIG. 13F) and response width after ultrasound stimulation in hsTRPA1 expressing neurons (FIG. 13G). FIG. 13H: Plot showing GCaMP6f peak amplitude in hsTRPA1 expressing neurons after ultrasound stimulation and treatment with either TRPV1 antagonist (A784168, 2 μM), Calcium chelator (BAPTA, 30 μM) or vehicle (DMSO). n=3 coverslips/condition. Numbers of cells analyzed is shown in each bar. *p<0.05, **p<0.01 by one-way ANOVA, (FIG. 13H) ****p<0.0001, n.s, not significant p>0.05 by Kruskal-Wallis rank test and Dunn's test for multiple comparisons.

FIGS. 14A-14L Electrophysiological properties of primary neurons. Functional and membrane properties are similar between TRPA1 and Cre-control infected neurons. Current-Voltage (IV) plots (FIG. 14A), for AAV9-hsTRPA1 versus AAV9-Cre control primary neurons elicit similar responses. Membrane resistance can be used as a proxy for patch and recording quality. FIG. 14B: Similar Rm was observed for both groups. Other response characteristics including inter-event interval (FIG. 14C) and response slopes (FIG. 14D) were not significantly altered between TRPA1 and Cre-control infected neurons. FIG. 14E: Relative response to ultrasound was significantly increased in TRPA1-expressing neurons, as was FIG. 14F: AUC of the response. N=5 cells/group. Ultrasound induced action potentials show similar metrics both in control and TRPA1 expressing neurons, including the peak voltage (FIG. 14G), latency relative to ultrasound stimulus (FIG. 14H) and time to peak (FIG. 14I).

FIGS. 15A-15F: myc-TRPA1 expresses in forelimb and hindlimb motor cortex, innervating lumbar and cervical spinal cord. FIG. 15A: Brain sections taken every ˜350 μM were immunolabeled for myc, GFP and DAPI to evaluate the rostro caudal extent of viral expression. Approximate AP coordinates are taken from Allen Brain Atlas (Sunkin, S. M. et al., Nucleic Acids Research 41, D996-D1008, doi:10.1093/nar/gks1042 (2012)). FIG. 15B: Spinal cord sections taken every ˜875 μM were immunolabeled for GFP and NeuN to evaluate the projection pattern of Npr3-Cre neurons that took up injected virus. Images are from a mouse that received co-injection of 4E13 myc-hsTRPA1 and 1 E12 GFP. Images were collected at 10×. FIG. 15C: A 20× confocal image of the inset from L5 showing GFP+ axons innervating the ventral horn. FIG. 15D: C6 spinal cord from the same mouse showing GFP+ axons in the ipsilateral (FIG. 15E) and contralateral (FIG. 15F) ventral horns.

FIGS. 16A-16J: Pressure-temperature profile of ultrasound delivery in vivo. FIG. 16A: Pressure profile of the ultrasound transducer used for in vivo experiments. Peak negative pressure was measured at a consistent location relative to the face of the transducer either through ultrasound gel, or in the cortex while the ultrasound transducer was coupled to the skull with ultrasound gel. Transducer pressure output increased as a function of changing the % gain on the amplifier. FIG. 16B: Peak temperature change measured 1 mm from the face of the transducer or in the cortex in response to 10 and 100 ms ultrasound stimulation at increasing pressures (reported pressures are those measured within the cortex). FIG. 16C: Representative temperature traces recorded within the cortex in response to stimulation at 0.70 MPa peak negative pressure at 10 or 100 ms stimulus durations. (FIG. 16c′) Inset from (FIG. 16C) showing the temperature rise during the stimulus. FIG. 16D: Schematic of hydrophone recordings in ex vivo mouse brain, with skull intact and palate removed. FIG. 16E: Dot in upper center indicates hydrophone location at optic chiasm, ventral-most part of the brain. Left upper dot and mid lower dots indicate subsequent measurements at constant power and variable depth. FIG. 16F: Transducer can deliver >1.5 MPa to deepest portions of the brain for sonogenetic applications. FIG. 16G: Representative midbrain coronal section, with dots representing hydrophone measurement locations. FIG. 16H: Ultrasound pressure delivered to midbrain; increased power can compensate for mid-range pressures. FIG. 16I: Representative hindbrain coronal section, with dots representing hydrophone measurement locations. FIG. 16J: Ultrasound pressure delivered to midbrain; increased power can compensate for mid-range pressures.

FIGS. 17A-17F: Data and Results from the in vivo experiments. FIG. 17A: Rotarod performance in mice injected with 1E12 AAV9-hSyn-DIO-GFP or 1E14 AAV9-hSyn-DIO-myc:TRPA1. N=6-7 per group. No significant differences were found between groups by two-way ANOVA and Sidak's multiple comparisons test. Both groups showed significant improvement in rotarod performance over the 5 days. P<0.0003 Day 5 compared to Day 1 by two-way ANOVA and Tukey's multiple comparisons test. FIG. 17B: Correlation between % c-fos+/GFP+ neurons and % myc+/GFP+ neurons across adjacent individual sections from mice that received ultrasound treatment. R2=0.626. P=0<0.001. Images of c-fos in the auditory cortex from myc-TRPA1-expressing mice that received sham stimulation (FIG. 17C) or 1 hour (1 hr) of 100 msec 1.05 MPa stimulation (FIG. 17D) delivered every 10 secs. FIG. 17E: Anatomical localization of auditory cortex in DAPI-labelled tissue. FIG. 17F: Quantification of % area of auditory cortex containing c-fos+ signal normalized to % area of the DAPI signal. No significant differences were detected across groups by One-way ANOVA.

FIGS. 18A-18D: The blood brain barrier is not disrupted by 1 hour of intermittent 100 ms ultrasound delivered at 1.0 MPa. Representative images of cortical fluorescent dextran (FIG. 18A) and mouse IgG immunolabeling across conditions (FIG. 18B). FIG. 18C: Quantification of 10 kDa fluorescent dextran in each cortical hemisphere from mice that were treated with either ultrasound (100 ms, 1.0 MPa every 10 s) or sham stimulation for 1 hour, or that had received a cortical stab wound condition, normalized to the cortical fluorescence of uninfected naïve mice. FIG. 18D: Quantification of mouse IgG in each cortical hemisphere from mice that were treated with either ultrasound (100 ms, 1.0 MPa every 10 s) or sham stimulation for 1 hour, or that had received a cortical stab wound condition, normalized to the cortical fluorescence of uninjected naïve mice. *p<0.05, **p<0.01, ****p<0.0001 by two-way ANOVA followed by Sidak's multiple comparison's test. N=3-5/group.

FIG. 19: Sonogenetics uses ultrasound (US) to non-invasively activate neurons. US can penetrate bone and tissue to perturb membranes in even the deepest part of the brain and has neuromodulatory effects. By expressing in cells, e.g., neuronal cells, an US-sensitive channel, termed Clone 63 herein, using a viral expression vector containing a polynucleotide sequence encoding the channel protein, namely, the Clone 63 channel protein, neuronal responsiveness to US stimulation was increased, leading to putative neuronal excitation via influx of cations.

FIG. 20 provides microscope images, diagrams, and graphs demonstrating that the US-sensitive channel protein Clone 63 confers US-sensitivity in neuronal cells (neurons) molecularly engineered to express Clone 63.

FIG. 21 provides microscope images and map graphs demonstrating that Clone 63 increases US responsiveness of primary neurons transfected with a vector (AAV) that expresses Clone 63 protein in the cells.

FIG. 22 presents images demonstrating that native Clone 63 (nonmutated) mediates an US-response in control neurons molecularly engineered to express native Clone 63 protein.

FIG. 23 presents schematic diagrams of brain, ultrasound-evoked electromyography (EMG) response readouts, graphs and microscope images demonstrating the sonogenetic activation of Layer V motor neurons molecularly engineered to express the Clone 63 channel protein.

FIG. 24 provides images of HEK cells expressing hsTRPA1 protein following transfection with an expression vector harboring hsTRPA1-encoding polynucleotide before and after ultrasound stimulation. Time-locked responses to a 100 ms 3 MPa 6.91 MHz pulse as assessed by GcaMP6f intensity are shown.

FIGS. 25A and 25B present a bar graph and microscope image. The graph in FIG. 25A shows the response to a 100 ms ultrasound pulse at 3 MPa 6.91 MHz in HEK cells transfected with different TRPA1 homologs. The bar plot presents the % of cells showing a response after ultrasound stimulation. N=3 coverslips/clone **p<0.01, ***p<0.001, ****p<0.0001. hs—Homo sapiens, cc—Castor canadiensis, ef—Eptesicus fuscus, vp—Vicugna pacos, ea—Equus asinus, dr—Danio rerio, pf—Poeciha formosa, am—Aligator mississippiensis. FIG. 25B shows a representative image of immunostaining against hsTRPA1 in hsTRPA1-expressing transfected HEK cells. Scale bar, 20 μm.

FIGS. 26A-26C present bar graphs. FIG. 26A presents a bar graph showing percent UV activation of cells molecularly engineered to express various TRPA1 mutant proteins or the human TRPA Clone 63 protein. FIG. 26B presents a summary bar graph showing GCaMP6f peak amplitude after ultrasound stimulation in cells transfected to express either wild type (WT/non-mutated) hsTRPA1 or representative mutant TRPA1 proteins. FIG. 26C presents a bar graph showing the % of cells showing a response after ultrasound stimulation for several different hsTRPA1 mutant proteins or the WT/non-mutated protein (Clone 63). The TRPA1 mutant proteins called mutant 7 and mutant 9 have modifications in ankyrin repeats, while the mutant 18 protein has modifications in the pore region. N=3 coverslips/clone. The number directly above each bar indicates the total number of cells (HEK cells transfected with hsTRPA1). Scale bar, 20 μm.

FIG. 27 presents a comparative alignment of amino acid residues in the relevant regions of the hsTRPA1 WT channel protein Clone 63 and the Mutant 18, Mutant 7 and Mutant 9 TRPA1 channel proteins. Differences between the amino acid sequences of the mutant proteins and the amino acid sequence of the WT hsTRPA1 Clone 63 protein are shown.

FIG. 28 presents tracings of ultrasound stimulation evaluated by patch clamp technique in excitable HEK cells expressing WT-Clone 63 channel polypeptide, the mutant 18 channel polypeptide, or control GFP polypeptide. Ultrasound stimuli is indicated by the light gray bar in the middle of the tracing.

FIG. 29 presents electromyography (EMG) traces of neurons expressing GFP, hsTRPA1 (native) and Mutant 18 (SonoChannel-1) in right forelimb motor cortex. Ultrasound stimulus is indicated by the lighter colored region.

FIGS. 30A-30C present a pictorial depiction, microscope images and a graph related to Mutant 18 TRPA1 channel polypeptide expression in dopaminergic neurons in the ventral tegumental area. FIG. 30A depicts that injecting a vector harboring mutant 18 TRPA1 channel polypeptide-encoding polynucleotide into the ventral tegumental area of mice, followed by ultrasound stimulation at the site, rendered the neurons sensitive to ultrasound. FIG. 30B shows microscope images of the neurons expressing Mutant 18 polypeptide following immunohistochemistry analysis. FIG. 30C: Monitoring c-fos-positive cells shows that significantly more neurons were activated by ultrasound when they expressed the mutant 18 channel polypeptide.

### BRIEF DESCRIPTION OF THE TABLES

Table 1 below shows a library of 89 clones from various protein families including DEG/ENaC, K2P, TRP, ASIC, Piezo, MscS, MscL and Prestin from multiple different species.

TABLE 1

| Protein Family | # Clones | # Species | Kingdom(s) |
|---|---|---|---|
| ASIC | 12 | 6 | Animals |
| KCNK | 2 | 1 | Animals (human) |
| MSCL | 13 | 4 | Bacteria |
| MSL | 4 | 1 | Plant |
| PIEZO | 8 | 2 | Animals (human, mouse) |
| PKD1 | 1 | 1 | Animals |
| Prestin | 7 | 5 | Animals |
| SCNN | 7 | 6 | Animals |
| TRPA1 | 10 | 10 | Animals |
| TRPC | 4 | 3 | Animals |
| TRPM | 9 | 4 | Animals |
| TRPN | 8 | 5 | Animals (zebrafish, cnidaria, drosophila) |
| TRPV | 8 | 5 | Animals (human, brown bat, killer whale) |
| Other | 99 | 21 | Animals, Plants, Bacteria |
| Total | 191 | 73 | |

Table 2 below shows the percent identity across all TRPA1 domains based on pair-wise alignment of consensus sequence for tested chordate, mammalian, and non-mammalian clades compared to human. Percent identity of A1-A16 in the table indicates regions that are particularly conserved or divergent between mammals and non-mammalian chordates. Threshold for consensus is bases matching to human reference in 65% of sequences in multiple sequence alignments of each clade.

TABLE 2

| | | | | | Percent |
|---|---|---|---|---|---|
| | | | Percent | Percent | Identity |
| | | | Identity | Identity | Human × |
| | | | Human × | Human × | Non- |
| | | | Chordate | Mammal | Mammal |
| | | Uniprot | Consensus | consensus | Consensus |
| ID | Domain | (start-stop) | 65% | 65% | 65% |
| TRPA1 | Protein | 1 | 65 | 79 | 46 |
| N | N-terminus | 1-61 | 25 | 58 | 13 |
| A1 | Ankyrin 1 | 62-92 | 44 | 46 | 32 |
| A2 | Ankyrin 2 | 97-126 | 73 | 87 | 58 |
| A3 | Ankyrin 3 | 130-160 | 50 | 67 | 36 |
| A4 | Ankyrin 4 | 164-193 | 61 | 71 | 45 |
| A5 | Ankyrin 5 | 197-226 | 66 | 69 | 52 |
| A6 | Ankyrin 6 | 238-267 | 69 | 72 | 53 |
| A7 | Ankyrin 7 | 271-301 | 78 | 84 | 60 |
| A8 | Ankyrin 8 | 308-337 | 74 | 80 | 66 |
| A9 | Ankyrin 9 | 341-370 | 82 | 97 | 55 |
| A10 | Ankyrin 10 | 374-403 | 71 | 90 | 49 |
| A11 | Ankyrin 11 | 412-441 | 82 | 93 | 52 |
| A12 | Ankyrin 12 | 445-474 | 92 | 97 | 74 |
| A13 | Ankyrin 13 | 481-510 | 94 | 100 | 71 |
| A14 | Ankyrin 14 | 513-542 | 84 | 93 | 57 |
| A15 | Ankyrin 15 | 547-576 | 74 | 88 | 61 |
| A16 | Ankyrin 16 | 579-609 | 58 | 80 | 44 |
| C1 | Cytoplasmic Domain 1 | 1-719 | NA | NA | NA |
| T1 | Transmembrane Domain 1 | 720-740 | 75 | 89 | 47 |
| E1 | Extracellular Domain 1 | 741-764 | 32 | 84 | 8 |
| T2 | Transmembrane Domain 2 | 765-785 | 59 | 86 | 26 |
| C2 | Cytoplasmic Domain 2 | 786-803 | 73 | 78 | 47 |
| T3 | Transmembrane Domain 3 | 804-824 | 68 | 73 | 34 |
| E2 | Extracellular Domain 2 | 825-829 | 62 | 62 | 5 |
| T4 | Transmembrane Domain 4 | 830-850 | 73 | 81 | 54 |
| C3 | Cytoplasmic Domain 3 | 851-873 | 67 | 96 | 53 |
| T5 | Transmembrane Domain 5 | 874-894 | 68 | 82 | 50 |
| E3 | Extracellular Domain 3 | 895-901 | 73 | 72 | 32 |
| P | Pore Region | 902-922 | 77 | 81 | 54 |
| E4 | Extracellular Domain 4 | 923-934 | 68 | 75 | 36 |
| T6 | Transmembrane Domain 6 | 935-956 | 61 | 78 | 38 |
| C4 | Cytoplasmic Domain 4 | 957-1119 | 59 | 74 | 45 |

### DETAILED DESCRIPTION OF THE DISCLOSURE AND EMBODIMENTS

Provided herein are compositions featuring TRPA1 polypeptides and polynucleotides, methods for expressing such polypeptides and polynucleotides in a cell type of interest, and methods for inducing the activation of the TRPA1 polypeptide in neurons and other cell types using ultrasound. In an embodiment, activation of the TRPA1 polypeptide in neurons and other cell types sensitizes the cell to ultrasound and can result in modulation or stimulation of cell function or activity. In an embodiment, the TRPA1 polypeptide expressed in neurons and other cell types is a heterologous or non-native protein.

Features of the aspects and embodiments described herein are based, at least in part, on the identification of human Transient Receptor Potential A1 (hsTRPA1), a channel polypeptide (protein) that confers sensitivity to non-invasive, low frequency ultrasound on millisecond timescales.

Our understanding of the nervous system has been fundamentally advanced by light- and small molecule-sensitive proteins that can be used to modify neuronal excitability. However, these require either invasive instrumentation or lack temporal control, respectively. Using a functional screen, it was found as described herein that human Transient Receptor Potential A1 (hsTRPA1) increased ultrasound-evoked intracellular calcium levels and membrane electrical events. hsTRPA1 ultrasound sensitivity, but not sensitivity to a chemical agonist, relied upon the N-terminal tip region, an intact actin cytoskeleton, and interactions with cholesterol, implicating these structures in the sonogenetic mechanism. Calcium imaging and electrophysiology were then used to confirm that primary neurons expressing hsTRPA1 potentiate their ultrasound-evoked responses. Finally, it was shown as described herein that inducing hsTRPA1 expression unilaterally in mouse layer V motor cortical neurons led to contralateral limb electromyography responses along with associated ‘twitch’ behaviors in response to ultrasound delivered through intact skull. Moreover, in some embodiments, ultrasound induced c-fos upregulation in hsTRPA1-expressing neurons, corroborating that these cells were selectively targeted. Together, the results described herein demonstrate the efficacy of sonogenetics for non-invasively modulating neurons within the intact mammalian brain, a method that could be extended to other cell types across species.

Accordingly, provided and featured herein are polynucleotides encoding a TRPA1 polypeptide, expression vectors comprising such polynucleotides, cells expressing a recombinant TRPA1 polypeptide, and methods for stimulating such cells with ultrasound. In an embodiment, the TRPA1 polypeptide is a human polypeptide.

### TRP4 and TRPA1

The present inventors previously showed that exogenous expression of the C. elegans TRP-4 mechanoreceptor enables ultrasound-sensitivity in neurons that are otherwise unresponsive to ultrasound stimulation. Similar ultrasound-sensitivity has also been observed in cells induced to express proteins belonging to the MSC, Piezo, Prestin, TRP, and TREK families in vitro. It was therefore hypothesized that mechanosensitive proteins would confer ultrasound sensitivity to mammalian cells and performed experiments to identify new ion channel family proteins possessing mechanosensitive properties that could confer ultrasound sensitivity to mammalian cells. In an embodiment, such mechanosensitive proteins confer ultrasound sensitivity to mammalian cells at higher frequencies. By way of example and without intending to be limiting, the frequencies used in studies with cells expressing the above-noted proteins was in the range of about 500 kHz-2 MHz, or 10 MHz, which may have limited spatial resolution and or require bulky transducers that restrict the ability to develop wearable devices. As described herein, higher ultrasound frequencies, e.g., greater than 2 MHz, e.g., 5-10 MHz may be used. For example, a frequency of 6.91 MHz is unlikely to induce cavitation, because its mechanical index range in the experiments described herein (0.37-0.95) is below the threshold value for cavitation onset of 1.9 in tissues.

A featured aspect as described herein was the identification and selection of new and beneficial ion channel polypeptides having mechanosensitive properties, wherein such polypeptides had the ability to confer ultrasound sensitivity to mammalian cells. In an embodiment, ultrasound sensitivity could be conferred to the cells at higher frequencies, e.g., 6.91 MHz, To identify the optimal candidate polypeptide, a functional readout-based assay was used to screen a library of more 191 putative mechanosensitive proteins and their homologs (Table 1). A combination of imaging, pharmacology, electrophysiology, and comparative sequence analysis, as well as behavioral, and histological analyses were used to demonstrate that a mammalian protein, Homo sapiens transient receptor potential A1 (hsTRPA1), conferred ultrasound sensitivity to cells in vitro and in vivo, establishing an advantageous sonogenetic tool, e.g., for use in mammals. In embodiments, an hsTRPA1 channel polypeptide as described herein encompasses an amino acid sequence of SEQ ID NOS: 4-7 or a functional fragment thereof.

### Ultrasound

Ultrasound is well suited for stimulating neuron populations as it focuses easily through intact thin bone and deep tissue (K. Hynynen and F. A. Jolesz, Ultrasound Med Biol 24 (2), 275 (1998)) to volumes of just a few cubic millimeters (G. T. Clement and K. Hynynen, Phys Med Biol 47 (8), 1219 (2002)). The non-invasive nature of ultrasound stimulation is particularly significant for manipulating vertebrate neurons including those in humans, as it eliminates the need for surgery to insert light fibers (required for some current optogenetic methods). Also, the small focal volume of the ultrasound wave compares well with light that is scattered by multiple layers of brain tissue (S. I. Al-Juboori, et al., PLoS ONE 8 (7), e67626 (2013)). Moreover, ultrasound has been previously used to manipulate deep nerve structures in human hands and reduce chronic pain (W. D. O'Brien, Jr., Prog Biophys Mol Biol 93 (1-3), 212 (2007); L. R. Gavrilov et al., Prog Brain Res 43, 279 (1976)). The aspects and embodiments described herein provide for novel non-invasive compositions for the expression of TRPA1 in cells, and methods to stimulate cells expressing TRPA1 using low-intensity ultrasound stimulation.

### Cellular Compositions Comprising Recombinant TRPA1

Provided and featured herein are cells comprising a recombinant nucleic acid molecule encoding a TRPA1 polypeptide. In one embodiment, a cardiac muscle cell comprising a TRPA1 polynucleotide under the control of a promoter suitable for expression in a cardiac cell (e.g., NCX1 promoter) is provided. In another embodiment, a muscle cell comprising a TRPA1 polynucleotide under the control of a promoter suitable for expression in a muscle cell (e.g., myoD promoter) is provided. In another embodiment, an insulin secreting cell (e.g., beta islet cell) comprising a TRPA1 polynucleotide under the control of a promoter suitable for expression in an insulin-secreting cell (e.g., Pdx1 promoter) is provided. In another embodiment, an adipocyte comprising a TRPA1 polynucleotide under the control of a promoter suitable for expression in an adipocyte (e.g., iaP2) is provided. Other embodiments provide a neuron or neuronal cell comprising a TRPA1 polynucleotide under the control of a promoter suitable for expression in a neuron (e.g., nestin, Tuj 1 promoter), in a motor neuron (e.g., H2b promoter), in an interneuron (e.g., Islet 1 promoter), in a sensory neuron (e.g., OMP promoter, T1R, T2R promoter, rhodopsin promoter, Trp channel promoter). The above-note promoters are provided by way of example and are not intended to be limiting. Such cells may be cells in vitro or in vivo.

### Expression of Recombinant TRPA1

In one approach, a cell of interest (e.g., a neuron, such as a motor neuron, sensory neuron, neuron of the central nervous system, e.g., an interneuron, or neuronal cell line) is molecularly engineered to express a TRPA1 polynucleotide whose expression renders the cell responsive to ultrasound stimulation. Ultrasound stimulation of such cells induces cation influx. Such cells express an heterologous or non-native TRPA1 polypeptide. In an embodiment, the expressed heterologous or non-native TRPA1 polypeptide is a human TRPA1 polypeptide (hsTRPA1) as described herein. In embodiments, the TRPA1 polypeptide comprises an amino acid sequence as set forth in any one of SEQ ID NOS: 1-3 or 4-7, or a functional fragment thereof.

TRPA1 may be constitutively expressed or its expression may be regulated by an inducible promoter or other control mechanism where conditions necessitate highly controlled regulation or timing of the expression of a TRPA1 protein. For example, heterologous DNA encoding a TRPA1 gene to be expressed is inserted in one or more pre-selected DNA sequences. This can be accomplished by homologous recombination or by viral integration into the host cell genome. The desired gene sequence can also be incorporated into a cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Calcium phosphate transfection can be used to introduce plasmid DNA containing a target gene or polynucleotide into cells and is a standard method of DNA transfer to those of skill in the art. DEAE-dextran transfection, which is also known to those of skill in the art, may be preferred over calcium phosphate transfection where transient transfection is desired, as it is often more efficient. Since the cells of the aspects and embodiments described herein are isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells can also be genetically modified using electroporation.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPA) can be added. Commercially available reagents for liposomal transfer include Lipofectin (Life Technologies). Lipofectin, for example, is a mixture of the cationic lipid N-[1-(2, 3-dioleyloxy)propyl]-N—N—N-trimethyl ammonia chloride and DOPE. Liposomes can carry larger pieces of DNA, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of DNA into primary and established mammalian cell lines using lipopolyamine-coated DNA can be used to introduce target DNA into the de-differentiated cells or reprogrammed cells described herein.

Naked plasmid DNA can be injected directly into a tissue comprising cells of interest. Microprojectile gene transfer can also be used to transfer genes into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors are used to genetically alter cells of the aspects and embodiments described herein, as well as their progeny. Viral vectors are used, as are the physical methods previously described, to deliver one or more polynucleotide sequences encoding TRPA1, for example, into the cells. Viral vectors and methods for using them to deliver DNA to cells are well known to those of skill in the art. Examples of viral vectors that can be used to genetically alter the cells as described herein include, but are not limited to, adenoviral vectors, adeno-associated viral vectors (AAV), such as AAV9, retroviral vectors (including lentiviral vectors), alpha-viral vectors (e. g., Sindbis vectors), and herpes virus vectors.

### Targeted Cell Types

TRPA1 can be expressed in virtually any eukaryotic or prokaryotic cell or cell line of interest. In one embodiment, the cell is a bacterial cell or other pathogenic cell type. In another embodiment, the cell is a mammalian cell, such as an adipocyte, muscle cell, cardiac muscle cell, insulin secreting cell (e.g., beta islet cell), or a neuronal or nerve cell (neuron). e.g., motor neuron, sensory neuron, neuron of the central nervous system, interneurons, primary neuron, and neuronal cell line. In an embodiment, the cell is a primary cell. In an embodiment, the cell is in vitro, ex vivo, in situ, or in vivo.

### Methods of Stimulating a Neural Cell

The methods provided herein are, inter alia, useful for the stimulation (activation) of cells. In particular, ultrasound stimulation induces cation influx, thereby altering cell activity. Expression of TRPA1 in a pathogen cell (bacteria) and subsequent ultrasound stimulation induces cation influx and bacterial cell killing. Ultrasound stimulation of a muscle cell expressing TRPA1 results in muscle contraction. This can be used to enhance muscle contraction or functionality in subjects in need thereof, including subjects suffering from muscle weakness, paralysis, or muscle wasting. Altering the intensity of the ultrasound modulates the extent of muscle activity.

The term “neural cell” as provided herein refers to a cell of the brain or nervous system. Non-limiting examples of neural cells include neurons, glia cells, astrocytes, oligodendrocytes and microglia cells. Where a neural cell is stimulated, a function or activity (e.g., excitability) of the neural cell is modulated by modulating, for example, the expression or activity of a given gene or protein (e.g., TRPA1) within said neural cell. The change in expression or activity may be 10%, 20%, 30%, 40%, 50%, 60%, 70%, 80%, 90% or more in comparison to a control (e.g., unstimulated cell). In certain instances, expression or activity is 1.5-fold, 2-fold, 3-fold, 4-fold, 5-fold, 10-fold or higher than the expression or activity in the absence of stimulation. In certain instances, expression or activity is 1.5-fold, 2-fold, 3-fold, 4-fold, 5-fold, 10-fold or lower than the expression or activity in the absence of stimulation. The neural cell may be stimulated by applying an ultrasonic wave to the neural cell.

The term “applying” as provided herein is used in accordance with its plain ordinary meaning and includes the meaning of the terms contacting, introducing and exposing. An “ultrasonic wave” as provided herein is an oscillating sound pressure wave having a frequency greater than the upper limit of the human hearing range. Ultrasound (ultrasonic wave) is thus not separated from ‘normal’ (audible) sound by differences in physical properties, only by the fact that humans cannot hear it. Although this limit varies from person to person, it is approximately 20 kilohertz (20,000 hertz) in healthy, young adults. Ultrasound (ultrasonic wave) devices operate with frequencies from 20 kHz up to several gigahertz. The methods provided herein use the energy of an ultrasonic wave to stimulate a neural cell expressing an exogenous mechanotransduction protein. A mechanotransduction protein as provided herein refers to a cellular protein capable of converting a mechanical stimulus (e.g., sound, pressure, movement) into chemical activity. Cellular responses to mechanotransduction are variable and give rise to a variety of changes and sensations. In embodiments, the mechanotransduction protein is a mechanically gated ion channel, which makes it possible for sound, pressure, or movement to cause a change in the excitability of a cell (e.g., a sensory neuron). The stimulation of a mechanotransduction protein may cause mechanically sensitive ion channels to open and produce a transduction current that changes the membrane potential of a cell.

In one aspect, a method of stimulating a cell is provided. The method includes (i) transfecting a cell with a recombinant vector including a nucleic acid sequence encoding an exogenous mechanotransduction polypeptide, thereby forming a transfected cell. (ii) To the transfected cell an ultrasonic wave is applied, thereby stimulating a cell. In embodiments, the mechanotransduction polypeptide is TRPA1 or a functional portion or homolog thereof. In embodiments, the ultrasonic wave has a frequency of about 0.8 MHz to about 4 MHz. In embodiments, the ultrasonic wave has a frequency of about 1 MHz to about 3 MHz. In embodiments, the ultrasonic wave has a focal zone of about 1 cubic millimeter to about 1 cubic centimeter.

In embodiments, the method further includes before the applying of step (ii) contacting the transfected neural cell with an ultrasound contrast agent. In embodiments, the ultrasound contrast agent is a microbubble. In embodiments, the microbubble has a diameter of about 1 μm to about 6 μm. In embodiments, the neural cell forms part of an organism. In embodiments, the organism is a bacterial cell or mammalian cell (e.g., human, murine, bovine, feline, canine).

### Methods of Treatment

In another aspect, a method of treating a neurological disease in a subject in need thereof is provided. The method includes (i) administering to a subject a therapeutically effective amount of a recombinant nucleic acid encoding an exogenous mechanotransduction polypeptide (e.g., TRPA1). In step (ii) an ultrasonic wave is applied to the subject, resulting in a change in TRPA1 conductance, i.e., cation influx. In one embodiment, the methods treat a cardiac disease by enhancing cardiac muscle activity or neurological disease by altering neural activity in the subject. In embodiments, the neurological disease is Parkinson Disease, depression, obsessive-compulsive disorder, chronic pain, epilepsy or cervical spinal cord injury. In embodiments, the neurological disease is retinal degeneration or atrial fibrillation. In embodiments, the mechanotransduction polypeptide is a TRPA1 polypeptide comprising an amino acid sequence as set forth in any one of SEQ ID NOS: 1-3 or SEQ ID NOS: 4-7 herein, or a functional fragment or portion thereof, e.g., an N-terminal fragment or portion. In an embodiment, the TRPA1 polypeptide is an hsTRPA1 polypeptide.

In embodiments, the mechanotransduction polypeptide is TRPA1 or a functional portion or homolog thereof. In embodiments, the mechanotransduction polypeptide is human TRPA1 or a functional portion or homolog thereof. In embodiments, the mechanotransduction polypeptide is human TRPA1 Clone 63 as described herein, or a functional portion or homolog thereof. In embodiments, the mechanotransduction polypeptide is a variant (mutant) TRPA1 polypeptide, or a functional portion or homolog thereof. In embodiments, the mechanotransduction polypeptide is a variant (mutant) of human TRPA1 Clone 63 as described herein, or a functional portion or homolog thereof. In embodiments, the mechanotransduction polypeptide is a human TRPA1 variant Mutant 7 as described herein, or a functional portion or homolog thereof. In embodiments, the mechanotransduction polypeptide is a human TRPA1 variant Mutant 9 as described herein, or a functional portion or homolog thereof. In embodiments, the mechanotransduction polypeptide is a human TRPA1 variant Mutant 18 as described herein, or a functional portion or homolog thereof. In an embodiment of the foregoing, the TRPA1, such as human TRPA1, or a functional portion or homolog thereof, recombinantly or molecularly expressed in a cell is a heterologous, non-native, non-naturally occurring, or exogenous polypeptide. In embodiments, the method further includes before the applying of step (ii) administering to the subject an ultrasound contrast agent. In embodiments, the ultrasound contrast agent is a microbubble. In embodiments, the microbubble has a diameter of about 1 μm to about 6 μm, and is injected into the body (e.g., the brain) where it enhances ultrasound stimulation.

### Pharmaceutical Compositions

The agents as described herein, e.g., TRPA1 polypeptides, polynucleotides encoding the TRPA1 polypeptides, and vectors, cells, and the like, comprising the foregoing, can be incorporated into a variety of formulations for therapeutic use (e.g., by administration) or in the manufacture of a medicament (e.g., for treating or preventing disease or disorder, such as a neurological disease or disorder) by combining the agents with appropriate pharmaceutically acceptable carriers, vehicles, or diluents, and may be formulated into preparations in solid, semi-solid, liquid or gaseous forms. Examples of such formulations include, without limitation, tablets, capsules, powders, granules, ointments, solutions, suppositories, injections, inhalants, gels, nanoparticles, microspheres, and aerosols.

Pharmaceutical compositions can include, depending on the formulation desired, pharmaceutically-acceptable, non-toxic carriers or diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents. toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents. stabilizers, and preservatives.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Pharmaceutical compositions can be prepared by any method known in the art of pharmacology. In general, such preparatory methods include the steps of bringing the agent (e.g., TRPA1, such as hsTRPA1) polypeptide, polynucleotide, or vector) described herein (i.e., the “active ingredient”) into association with a carrier or excipient, and/or one or more other accessory ingredients, and then, if necessary and/or desirable, shaping, and/or packaging the product into a desired single- or multi-dose unit. Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The agents and compositions provided herein can be administered by any route, including enteral (e.g., oral), parenteral, intravenous (into the CNS), intracerebroventricular, intramuscular, intra-arterial, intramedullary, intrathecal, subcutaneous, intracranial, ocular/intraocular, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation. Specifically contemplated routes are intravenous administration (e.g., systemic intravenous injection), regional administration via blood and/or lymph supply, and/or direct administration to an affected site. In general, the most appropriate route of administration will depend upon a variety of factors including the nature of the agent and/or the condition of the subject (e.g., whether the subject is able to tolerate a given route of administration). In certain embodiments, the agent or pharmaceutical composition described herein is suitable for topical administration to the eye of a subject.

### Kits

In one aspect, a kit for producing mechanosensitivity or sensitivity to ultrasound so as to modify the function or activity of a cell, a cell in a subject, or in a subject, e.g., ex vivo, in vitro, or in vivo, are provided. For example, the kit can be used to introduce into a subject or into a cell heterologous (non-native) TRPA1 polypeptide as described herein, e.g., hsTRPA1, so as to modulate or stimulate the activity of the cell following exposure to ultrasound. In an embodiment, the TRPA1 polypeptide is an exogenous human polypeptide. In an embodiment, the human TRPA1 polypeptide is the Clone 63 polypeptide as described herein. In embodiments, the TRPA1 polypeptide is a variant polypeptide, e.g., the mutant 18, 9, or 7 polypeptides as described herein. In embodiments, the kit comprises a vector, for example, without limitation, a viral vector, containing a polynucleotide encoding the TRPA1 polypeptide as described herein for introduction, e.g., by infection, injection, transfection, or transduction, into a cell, cell line, or a subject, wherein the TRPA1 polypeptide is expressed as a heterologous (non-native) channel protein in the cell or the subject. If desired, the kit may include a polynucleotide encoding a TRPA1 polypeptide or a TRPA1 polypeptide as described herein. In an embodiment, the kit may include a reporter or detection molecule (e.g., a detectably labeled molecule) to assess the expression of the TRPA1-encoding polynucleotide or the TRPA1 polypeptide in a cell or subject.

The kit may include instructions for the assay, reagents, testing equipment (test tubes, reaction vessels, needles, syringes, etc.), standards for calibrating the assay, and/or equipment provided or used to conduct the assay. The instructions provided in a kit according to the invention may be directed to suitable operational parameters in the form of a label or a separate insert.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology;” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides as described herein, and, as such, may be considered in making and practicing the various aspects and embodiments as described. Particularly useful techniques for particular embodiments will be discussed in the sections that follow.

The following examples are put forth so as to provide those of ordinary skill in the art with a complete disclosure and description of how to make and use the products, compositions, cells, vectors, therapeutic methods and the like as described herein, and are not intended to limit the scope of the aspects and embodiments described.

### EXAMPLES

### Example 1: Human Transient Receptor Potential A1 (hsTRPA1) Identified as a Sonogenetic Candidate

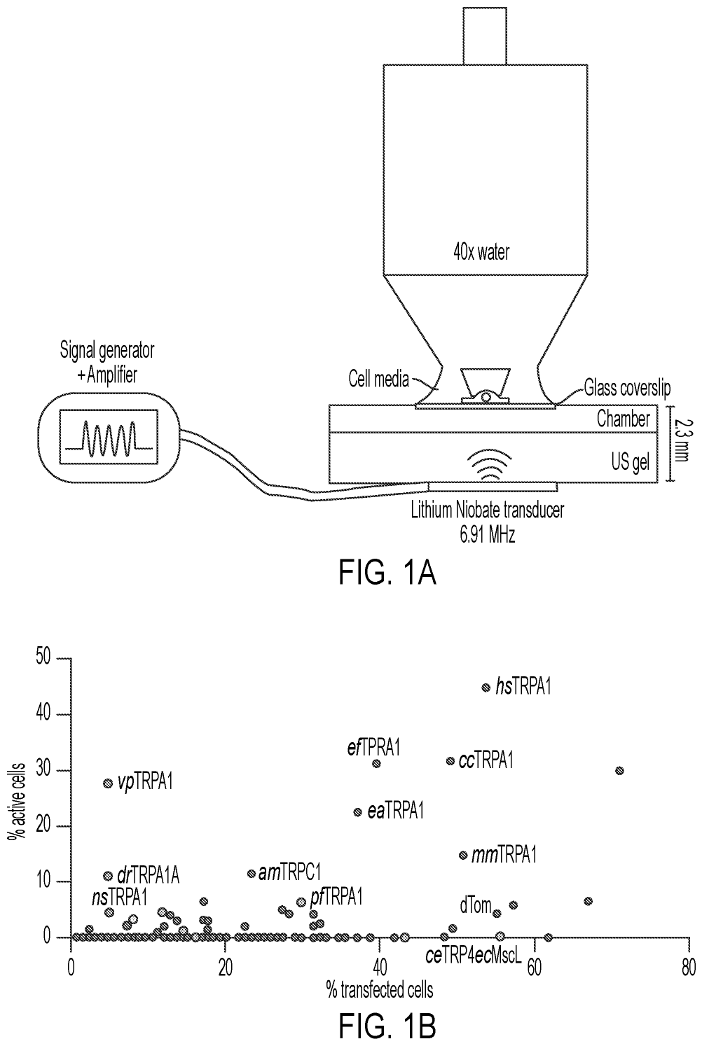

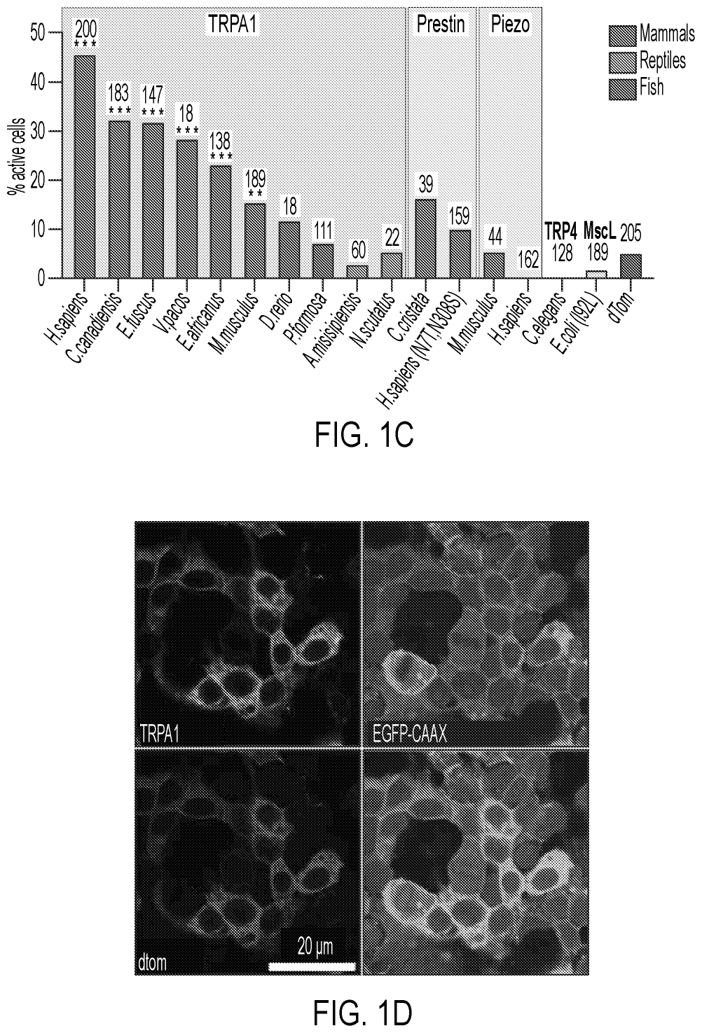

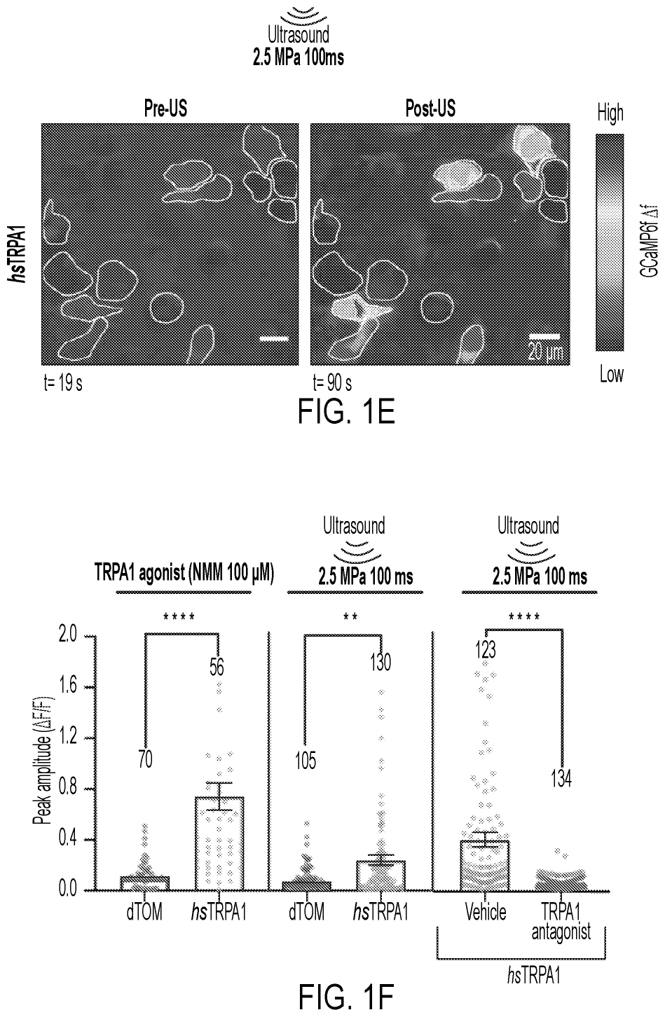

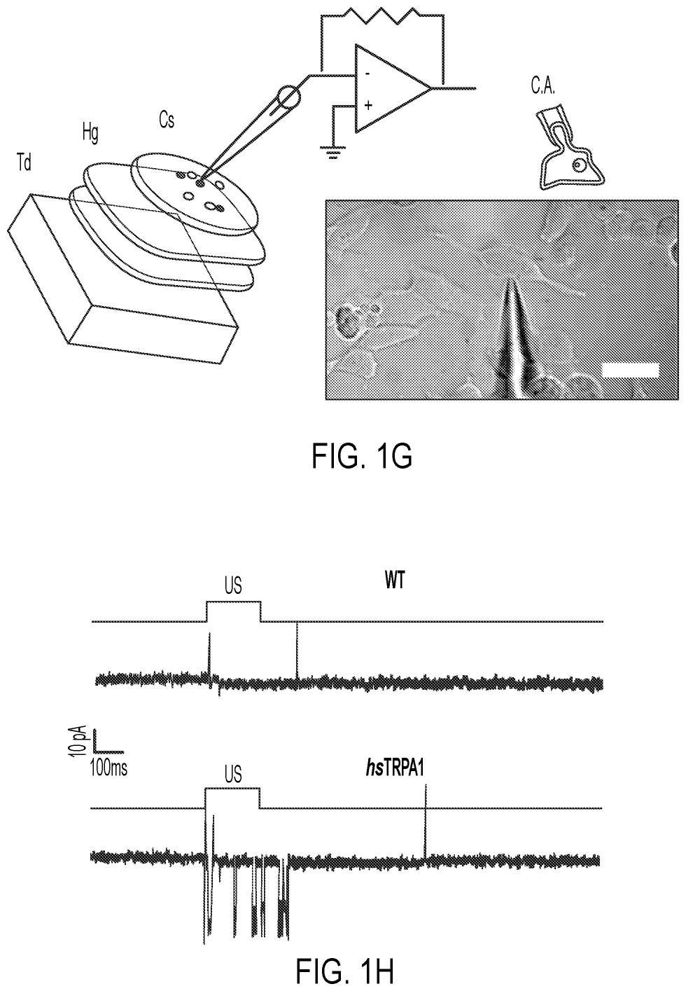

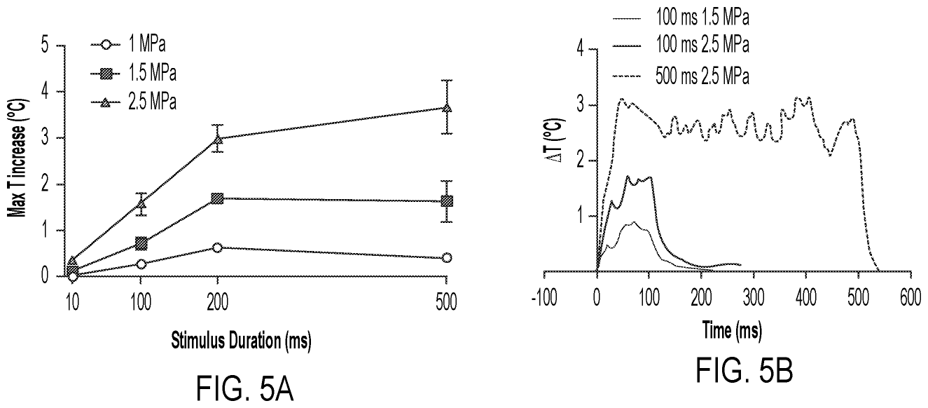

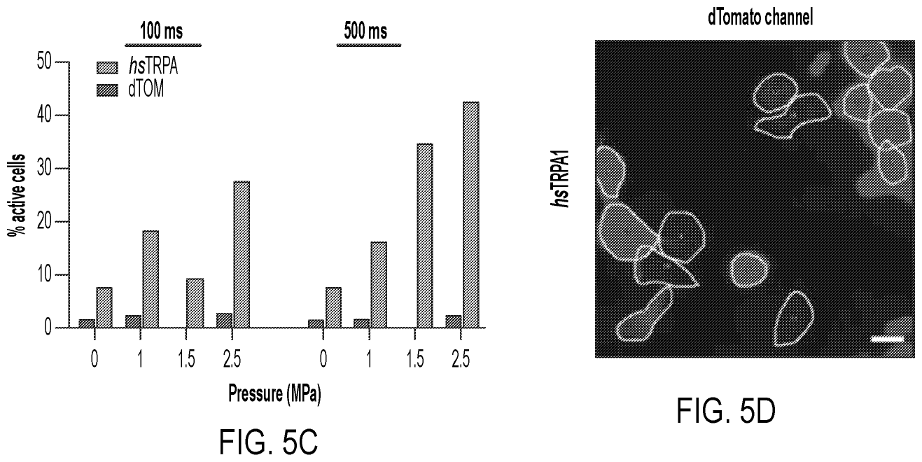

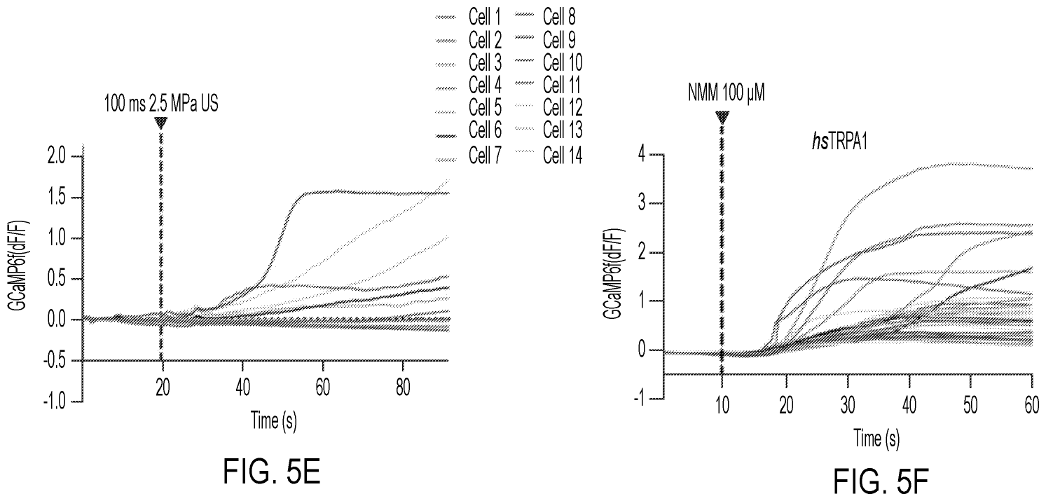

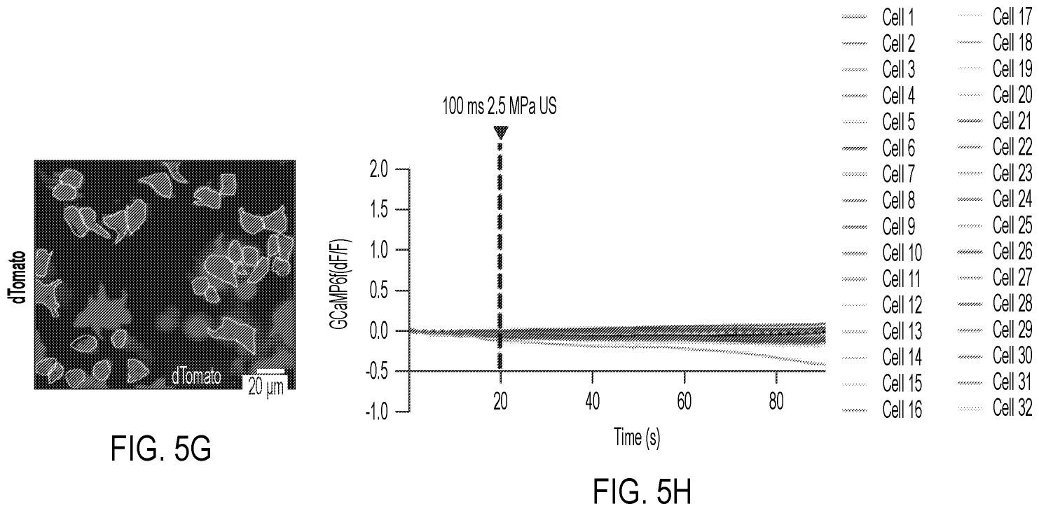

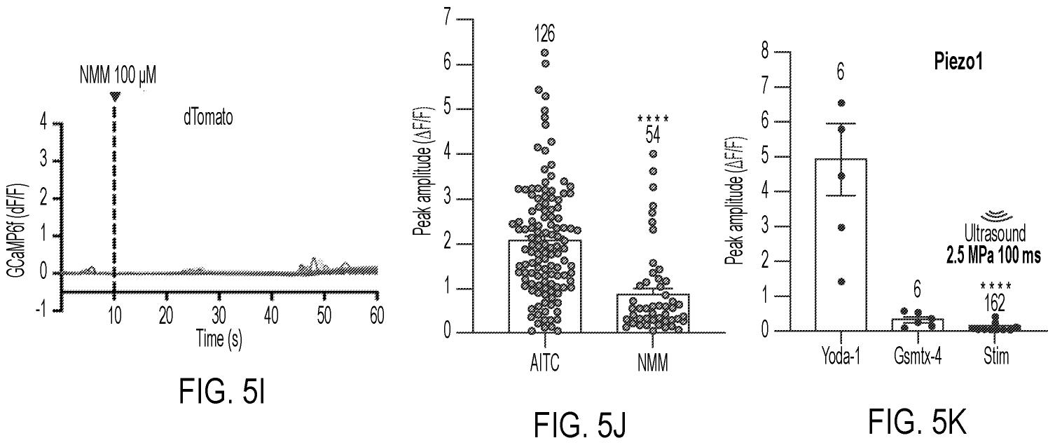

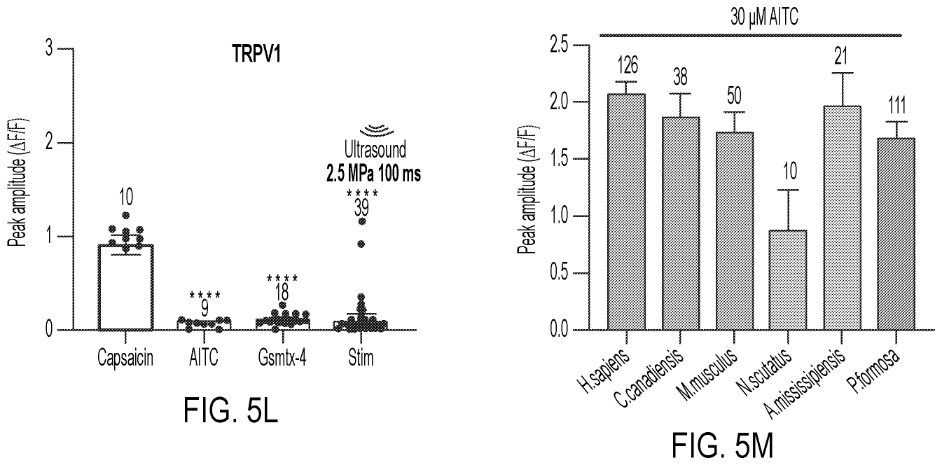

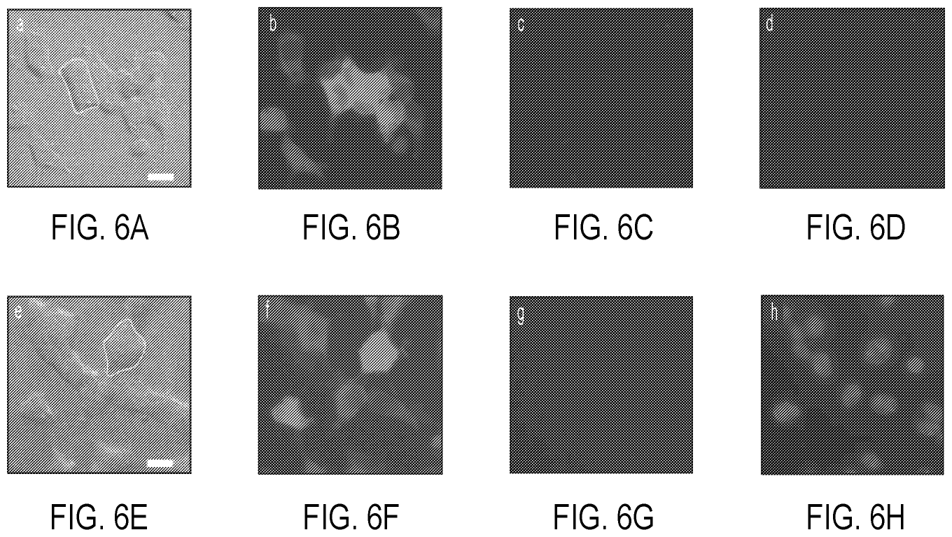

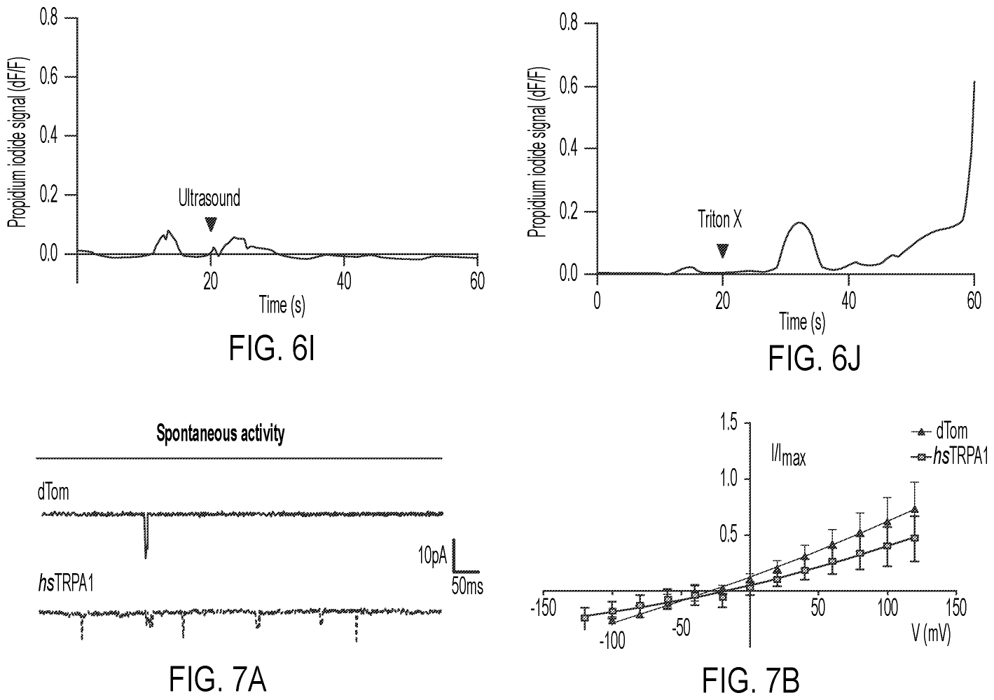

To facilitate rapid screening of ultrasound-triggered cellular responses, an optical imaging setup was aligned with a custom designed transducer. In particular, a single-crystal 6.91 MHz lithium niobate transducer (FIG. 1A) that lacks hysteresis and generates minimal heat as it converts electrical input into mechanical energy was designed. Such a transducer reduced ultrasound-triggered temperature changes. The pressure output and corresponding temperature changes in the imaging set up (or cell culture dish) were also profiled using a combined fiber optic probe (FIGS. 5A and 5B) and identified ultrasound parameters for the screen at a pressure and duration (100 msecs, 1.5 MPa) that caused minimal temperature change. Next, a dye-labelling approach was used to test if these ultrasound stimulus parameters were affecting the integrity of the plasma membrane. It was found that propidium iodide was unable to penetrate the cells exposed to ultrasound stimuli, confirming that these parameters were not disrupting cellular membranes (FIGS. 6A-6J).

We transiently transfected each candidate protein along with a fluorescent reporter (dTomato—dTom) into Human Embryonic Kidney 293T (HEK) cells expressing a genetically encoded calcium indicator (GCaMP6f) and monitored calcium changes upon ultrasound stimulation. (FIGS. 5D, 5E, 5G and 5H). It was found as described herein that cells expressing mammalian TRPA1 channels responded to ultrasound most frequently, ant that the human homolog was the best candidate (FIG. 1B). Moreover, a significant fraction of the hsTRPA1-expressing cells had robust responses (FIGS. 5A-5M). In contrast, the mouse homolog was only a third as responsive as hsTRPA1, and non-mammalian variants were insensitive to ultrasound (FIG. 1C). None of the other candidates tested in the screen showed significant sensitivity to the ultrasound parameters used in the screen, including channels previously shown to respond to ultrasound stimuli at different frequencies, pressures, or durations (FIG. 1C). While functional expression of Piezol and TRPV channels was confirmed (FIGS. 5K and 5L), issues with expression, trafficking, or folding may have affected the performance of the other candidate proteins.

Next, experiments were conducted to confirm that ultrasound responsiveness was due to direct activation of hsTRPA1. hsTRPA1 was first visualized using immunohistochemistry. It was found that hsTRPA1 protein was indeed expressed only in dTom+ cells and trafficked to the HEK cell membranes (FIG. 1D), where it co-localized with a membrane-labelled CAAX-GFP. Because a small fraction of the hsTRPA1 was detected on the membrane, it cannot be ruled out that the hsTRPA1 protein plays a role in other cellular compartments. Consistently, it was found as described herein that HEK cells expressing hsTRPA1 were selectively activated by ultrasound stimulation in a pressure- and duration-dependent manner (FIG. 5C), while dTom control HEK cells showed no response to ultrasound stimulation (FIGS. 5G and 5H). Moreover, the results showed that the TRPA1-selective agonists, N-methylmaleimide (NMM) and allyl isothiocyanate (AITC), also specifically activated TRPA1-expressing HEK cells, confirming that the channel was indeed functional (FIG. 1F and FIGS. 5F, 5I and 5J). Ultrasound responses also were inhibited when hsTRPA1-expressing cells were incubated with a TRPA1 antagonist, HC-030331 (FIG. 1F). Collectively, these results show that ultrasound responses require gating of hsTRPA1, which facilitated an increase in intracellular calcium.

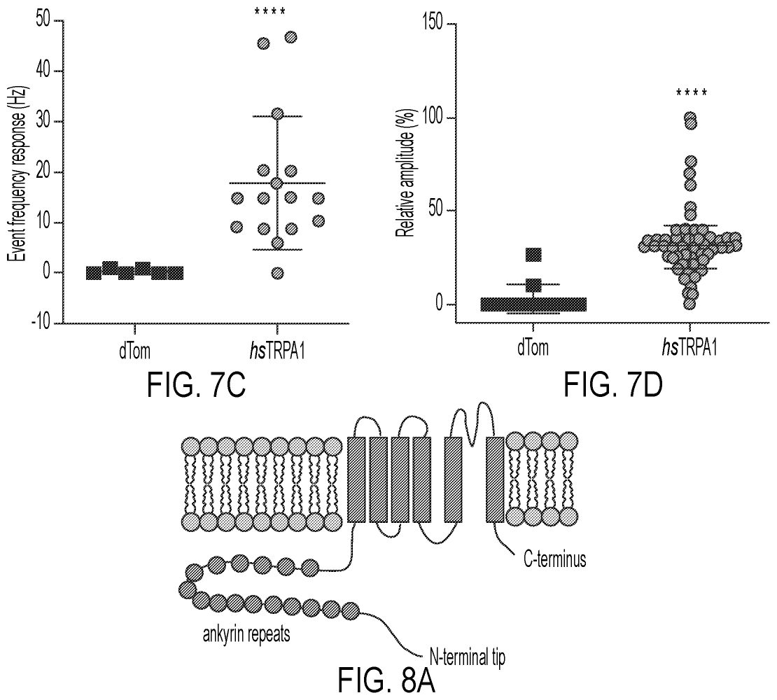

Next, electrophysiological methods were used to monitor changes in the membrane conductance of excitable HEK cells expressing hsTRPA1 or dTom-only control. To increase the recording efficiency, a cell-attached configuration (FIG. 1G) was used, wherein suitable access and membrane resistance were able to be maintained while the cells were exposed to ultrasound stimuli. It was found that cells expressing hsTRPA1 had had higher basal rates of activity (FIG. 7A) but had no significant disruptions in their electrical properties (I-V curve) compared to controls (FIG. 7B), confirming that channel expression did not alter membrane properties. Furthermore, inward currents in response to ultrasound were significantly larger and more numerous in hsTRPA1-expressing cells compared to those of controls (FIG. 1H and FIGS. 7C, 7D). These ultrasound-triggered currents were of similar magnitude as those previously observed for pharmacological activation of the TRPA1 channel (Wang, Y. Y. et al., J Biol Chem 283, 32691-32703, doi:10.1074/jbc.M803568200 (2008)). Furthermore, it was found herein that the ultrasound-triggered membrane events were attenuated by the presence of the TRPA1 antagonist, HC-030301 confirming a specific role for hsTRPA1 (FIG. 1I). Taken together, these results show that short ultrasound pulses can selectively lead to opening of hsTRPA1 channels, resulting in a rapid increase of intracellular calcium in HEK cells.

### Example 2: Putative Mechanisms Underlying Ultrasound Sensitivity of TRPA1

Multiple studies have shown that TRPA1 is a widely conserved calcium permeable, non-selective cation channel that is involved in detecting a wide-range of exogenous stimuli, including electrophilic compounds that interact with the nucleophilic amino acids in the channel, small peptides that partition in the plasma membrane, cold, heat, and others, although sensitivity to different stimuli varies across species. Despite this broad sensitivity and a resolved crystal structure, the underlying mechanisms of TRPA1 activation are only recently being discovered. For example, a scorpion toxin peptide (WaTx) has been shown to activate TRPA1 by penetrating the lipid bilayer to access the same amino acids bound by electrophiles, thereby stabilizing the channel in an active state and prolonging channel opening (King, J. V. L. et al., Cell, 178, 1362-1374. e1316 (2019)). In contrast, electrophilic irritants have been shown to activate the TRPA1 channel using a two-step cysteine modification that widens the selectivity filter to enhance calcium permeability and open the cytoplasmic gate. These studies suggest that the TRPA1 channel might interact with the cytoskeleton and components of the membrane bilayers, including cholesterol, to transduce signals into a cell.

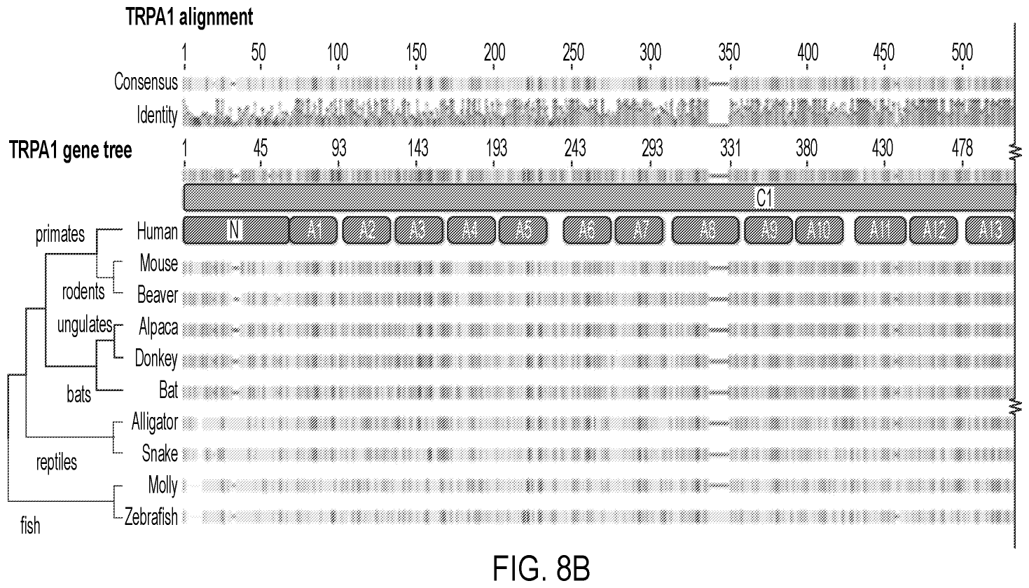

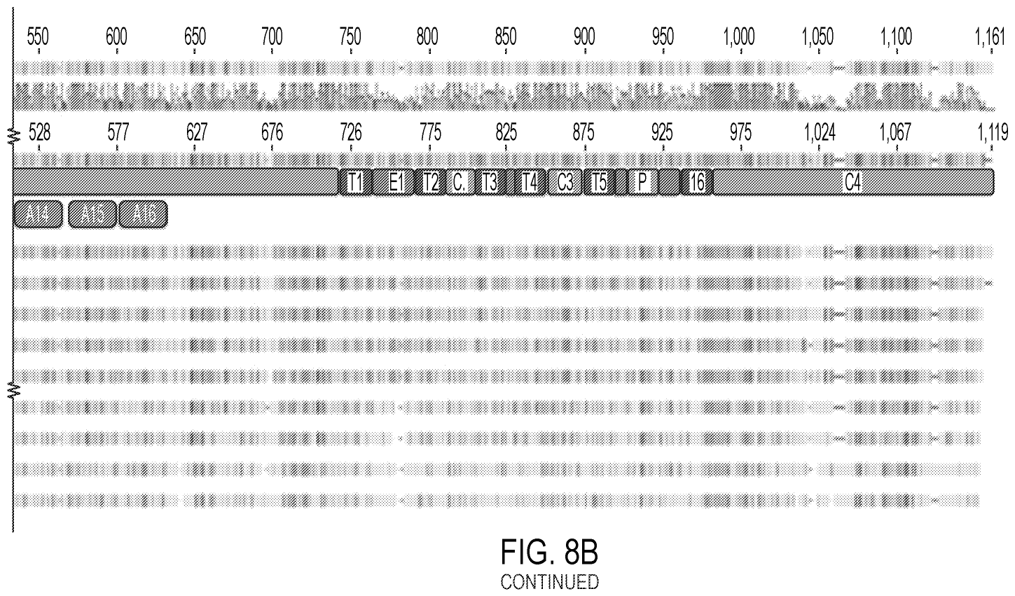

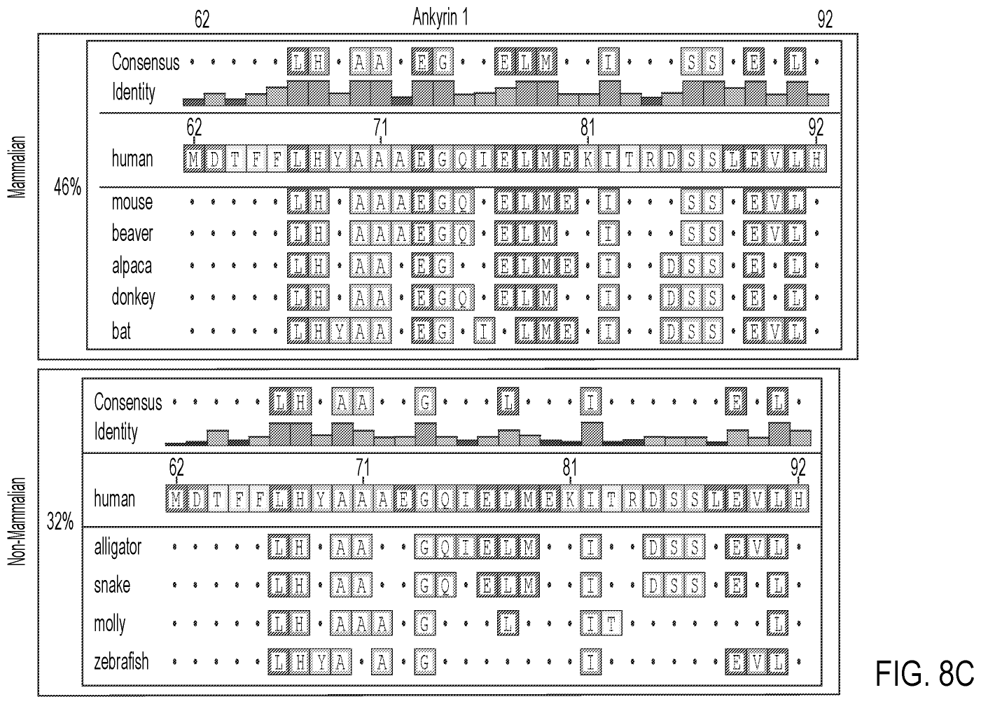

Structurally, TRPA1 comprises an intracellular N-terminal tip domain, 16 ankyrin repeats, 6 transmembrane domains and an intracellular C-terminal domain (FIG. 8A). To identify TRPA1 domains critical for ultrasound sensitivity, sequences of each domain in the human protein were compared to its ultrasound-sensitive mammalian and ultrasound-insensitive non-mammalian chordate TRPA1 homologs (FIG. 8B and Table 2). It was predicted that hsTRPA1 domains and motifs conserved among mammals are crucial to ultrasound sensitivity.

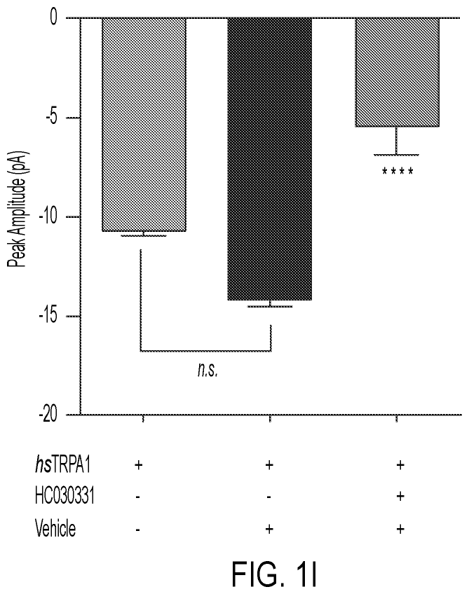

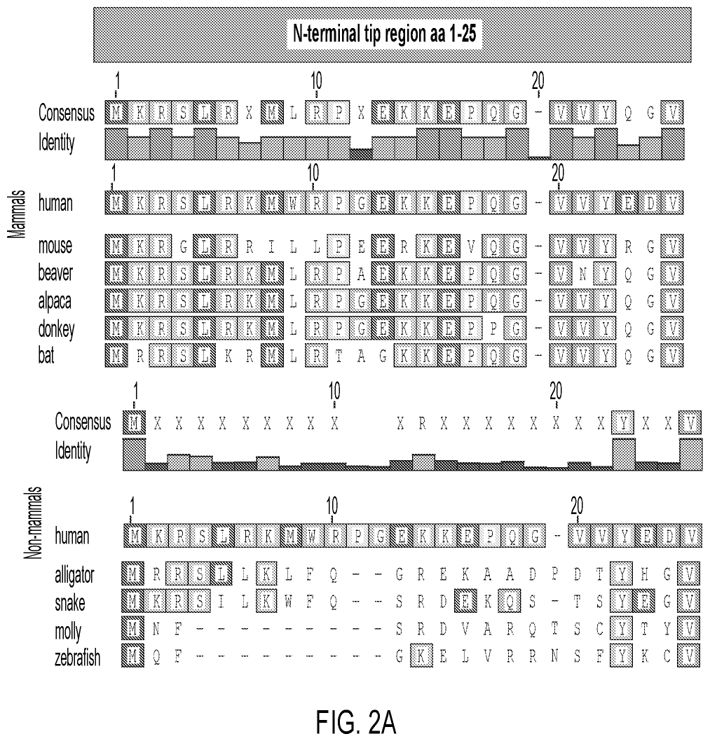

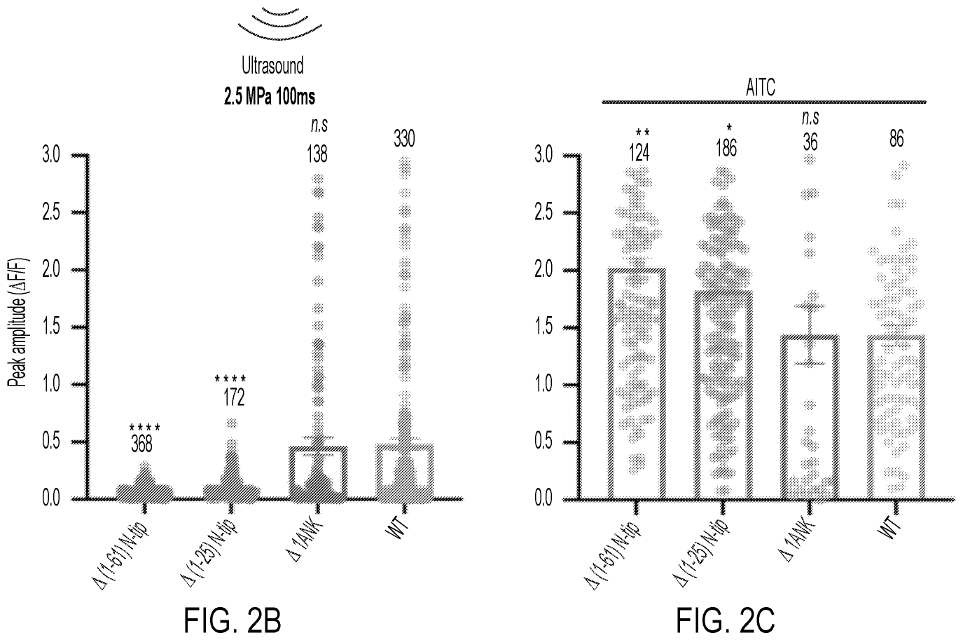

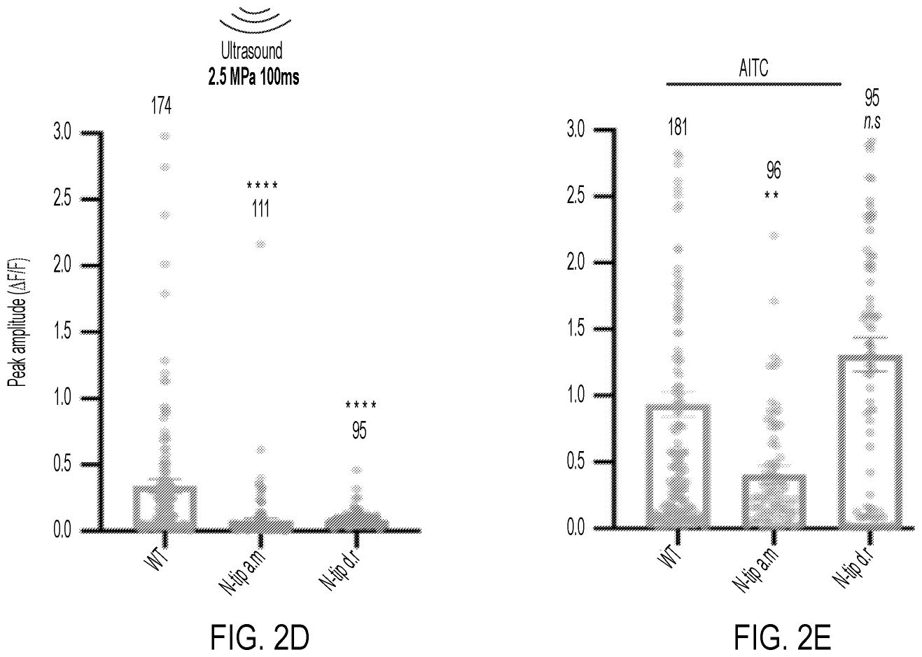

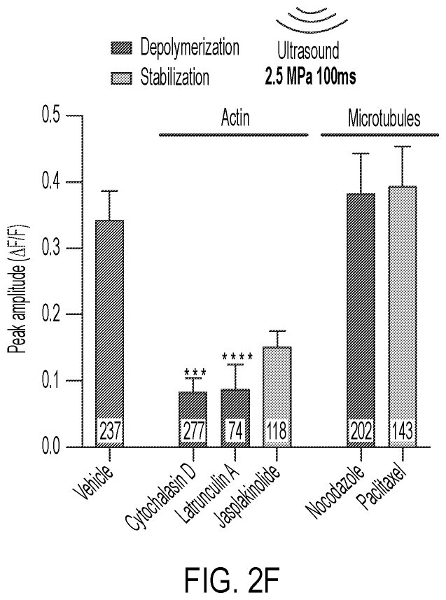

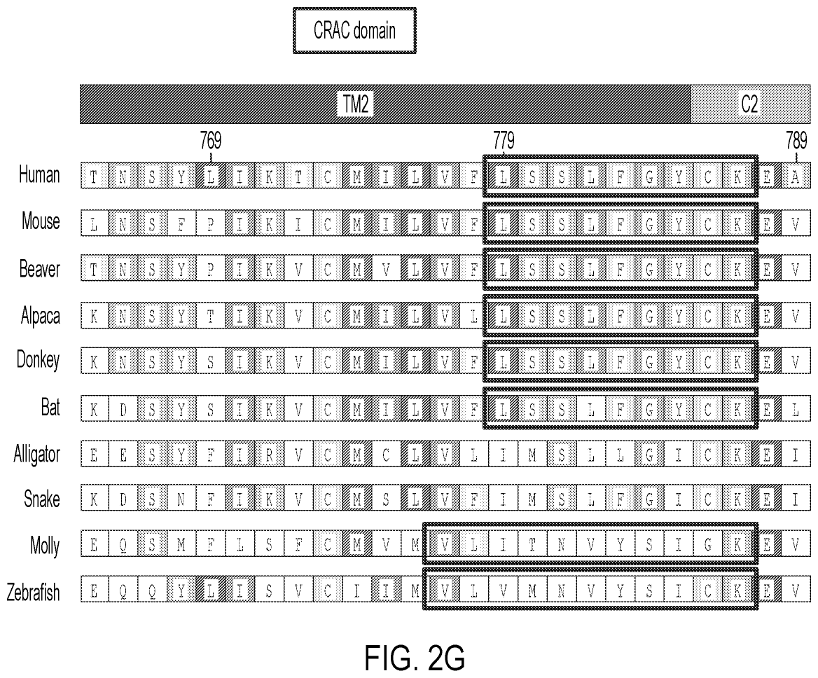

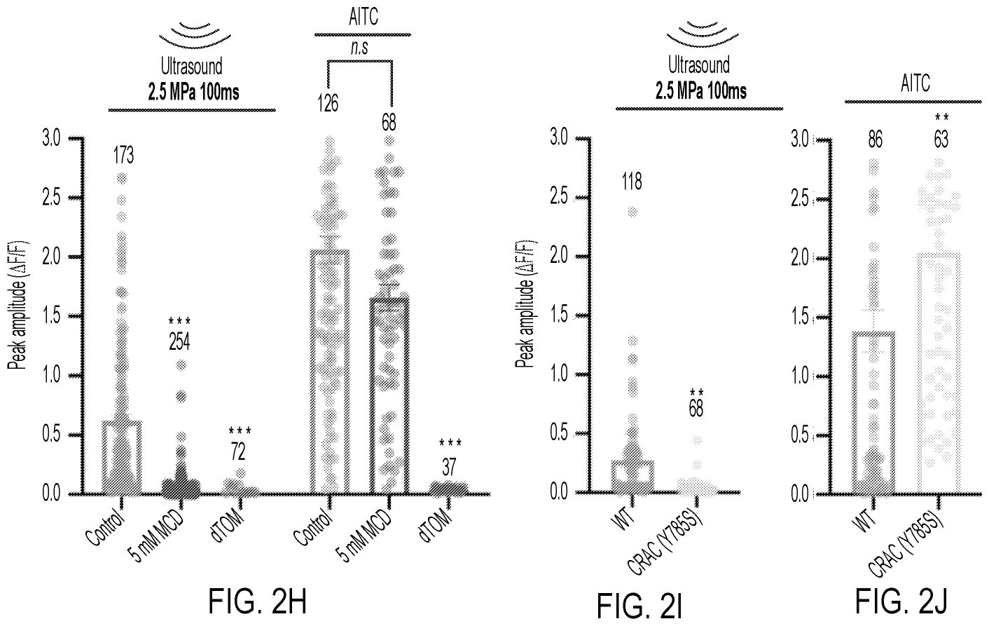

Sequence analysis of hsTRPA1 and its homologs (9 additional homologs) revealed that the 61 amino acid N-terminal tip region is highly conserved in mammalian compared to non-mammalian chordate species (58% vs 13% identity, respectively), particularly the first 22 amino acids (87% vs 17% identity, respectively FIG. 2A). Therefore, it was hypothesized that the N-terminal tip region might be important for mediating ultrasound sensitivity. Indeed, deletion of the entire N-terminal tip region (Δ1-61) and or the most highly conserved portion (Δ1-25) from hsTRPA1 completely abolished responses to ultrasound (FIG. 2B), while significantly increasing sensitivity to chemical agonist (FIG. 2C).

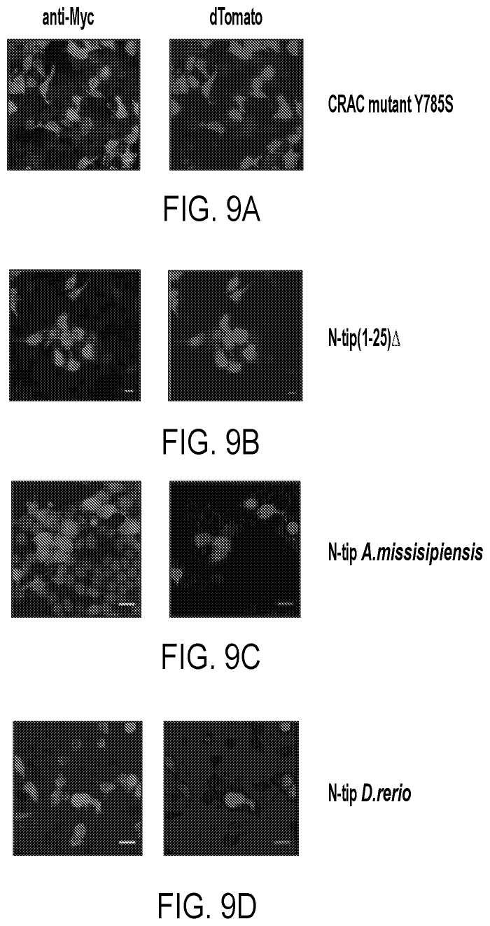

In contrast to the N-terminal tip region, the ankyrin repeat regions are highly conserved across both mammals (82% identity) and non-mammalian species (54% identity), with the exception of ankyrin 1, which is least conserved across mammals (46% identity; Table S2 and FIG. 8C). It was therefore hypothesized that ankyrin 1 would not be required for the ultrasound response. Indeed, deletion of only the first ankyrin repeat (ΔANK1) had no effect on either sensitivity to ultrasound or the chemical agonist (FIG. 2B, FIG. 2C). In order to further confirm the importance of the human N-terminal tip for mediating ultrasound sensitivity, chimeras containing the alligator or zebrafish N-terminal tip swapped into hsTRPA1 were created. These chimeras completely lost the ability to respond to ultrasound (FIG. 2D). However, the alligator/hsTRPA1 chimera had attenuated responses to AITC (FIG. 2E), suggesting that this channel may also have altered functionality. In contrast, the zebrafish/hsTRPA1 chimera had normal responses to AITC (FIG. 2E). Nevertheless, immunohistochemistry showed comparable expression and trafficking of mutated channels, indicating that their lack of ultrasound responses is not a consequence of poor expression (FIGS. 9A-9D). Taken together, the results suggest that the human N-terminal tip region is important for hsTRPA1 ultrasound sensitivity.

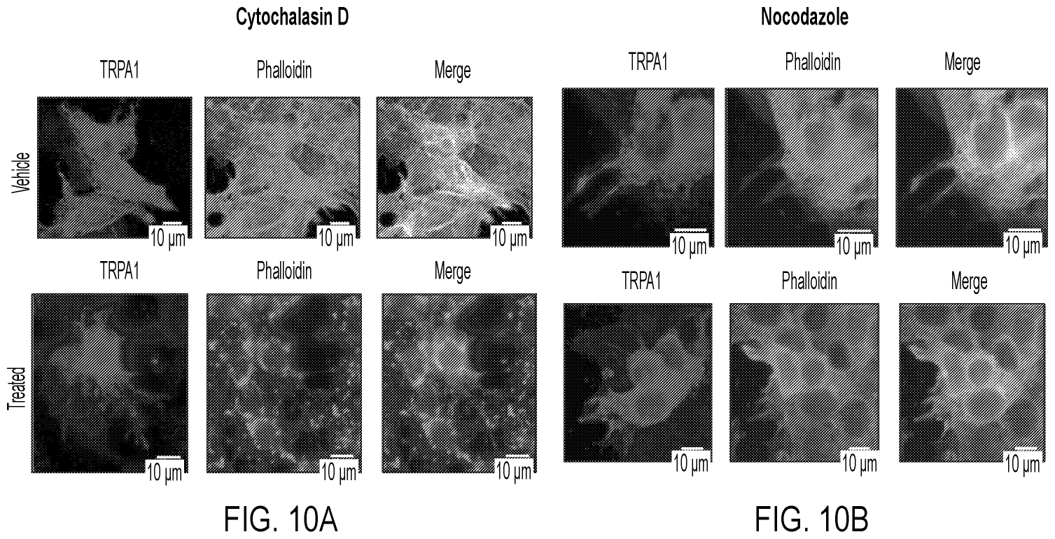

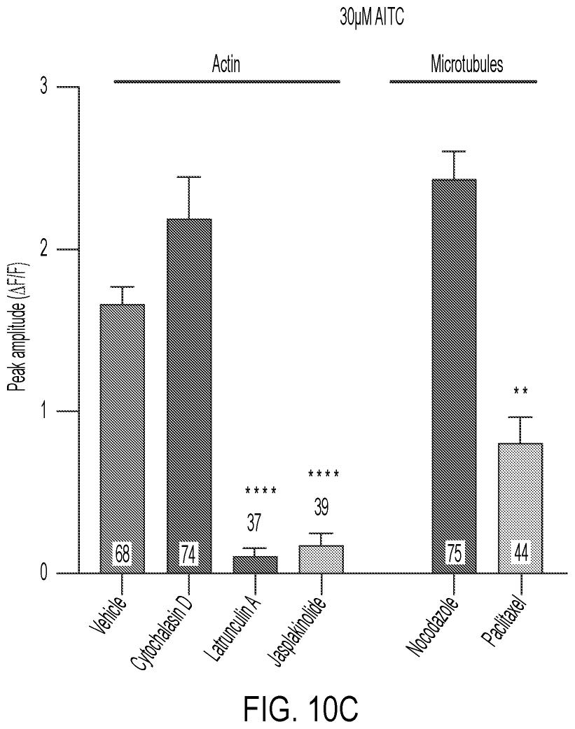

The N-terminal ankyrin repeats have also been hypothesized to interact with cytoskeletal elements and act as a gating spring in response to mechanical stimuli. For example, ankyrin repeat regions from Drosophila NOMPC (TRPN) are thought to be important in mechanosensation due to their interactions with microtubules. Therefore, experiments were performed as described herein to assess the involvement of cytoskeletal elements in ultrasound sensitivity of hsTRPA1. It was found that treating hsTRPA1-expressing HEK cells with the actin depolymerizing agents cytochalasin D and latrunculin A reduced the ultrasound responses of these cells compared to vehicle or an actin stabilizing agent, jasplakinolide (FIG. 2F). In contrast, disrupting or stabilizing microtubules with nocodazole or Taxol, respectively, had no significant effect on ultrasound-evoked hsTRPA1 responses (FIG. 2F). Immunohistochemistry confirmed that destabilizing treatments did indeed disrupt the actin cytoskeleton and microtubules (FIGS. 10A and 10B). Moreover, AITC-triggered responses were found not to be altered by treatment with either cytochalasin D or nocodazole, although they were significantly reduced by latrunculin A, jasplakinolide, and paclitaxel, confirming that these treatments did not completely disrupt TRPA1 function (FIG. 10C). Therefore, actin depolymerization by cytochalasin D treatment selectively blocked hsTRPA1 responses to ultrasound, but not chemical agonist, demonstrating a specific role for the actin cytoskeleton in ultrasound sensation.

Mouse TRPA1 has also been hypothesized to localize to lipid rafts through a mechanism governed by a Cholesterol Recognition/interaction Amino acid Consensus sequence (CRAC) domain within the transmembrane helix 2 (TM2) of TRPA1. Interestingly, as described herein, a CRAC motif (L/V-(X)(1-5)-Y-(X)(1-5)-R/K, where X are non-polar residues) was identified in transmembrane helix 2 of TRPA1 that was highly similar in all mammalian homologs tested, but was absent in reptiles and heavily modified in fish (FIG. 2G). Therefore, it was hypothesized that interactions with cholesterol might be important for ultrasound responsiveness of hsTRPA1 channels. To assess this, cellular membrane cholesterol was depleted with methyl-beta-cyclodextrin (MCD). It was found as described herein that this treatment attenuated hsTRPA1 responses to ultrasound, but did not affect the response to AITC (FIG. 2H). In order to further explore the importance of the TM2 CRAC motif, a mutant channel was created in which the required central amino acid residue tyrosine was replaced with a serine residue. This mutation caused a complete loss of ultrasound sensitivity without affecting responsiveness to AITC (FIGS. 2I and 2J), thus confirming the criticality of this TM2 CRAC motif. While the precise molecular mechanism of TRPA1 responsiveness to ultrasound remains elusive, the studies described herein suggest a role for the N-terminal tip region, the actin cytoskeleton, and interaction with cholesterol in driving ultrasound-evoked hsTRPA1 responses.

### Example 3: Primary Neurons Expressing hsTRPA1 are Ultrasound-Sensitive

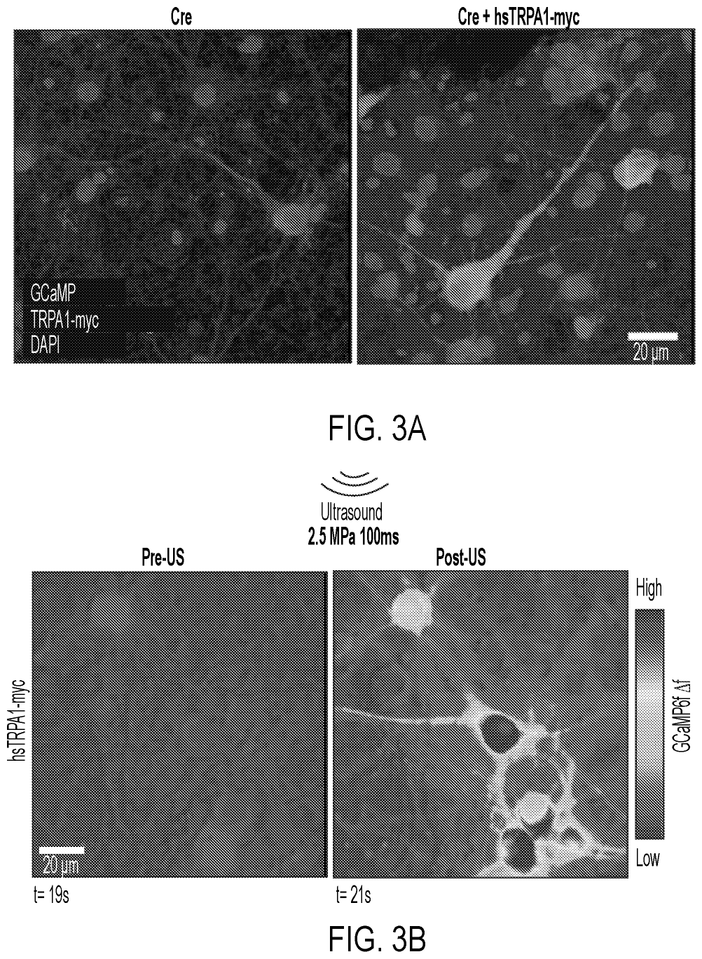

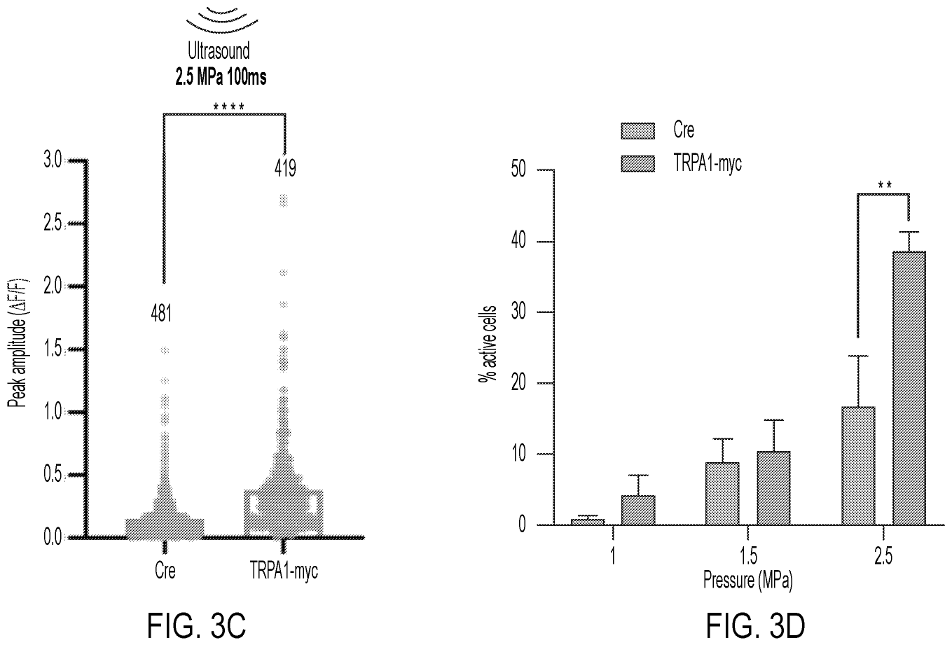

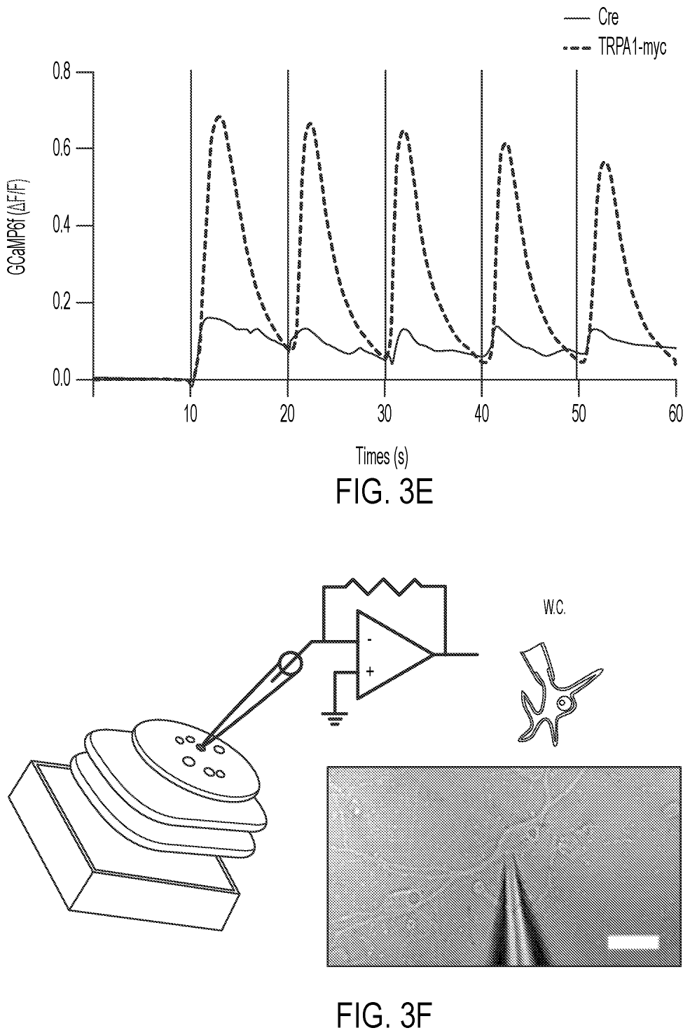

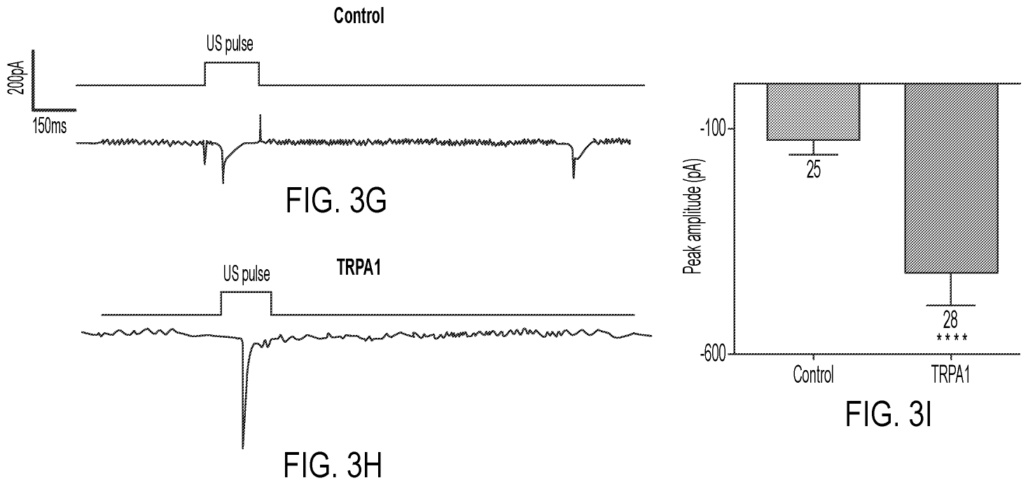

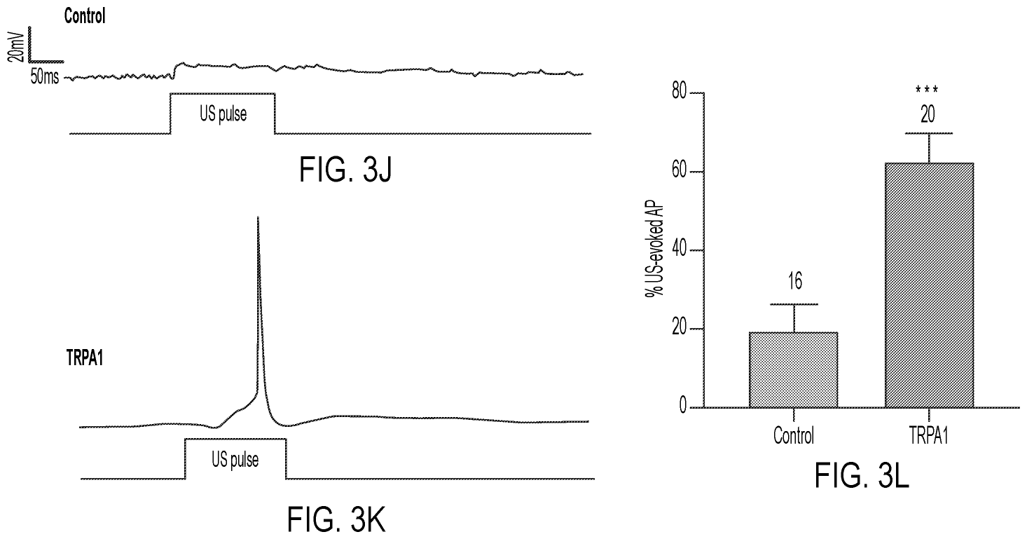

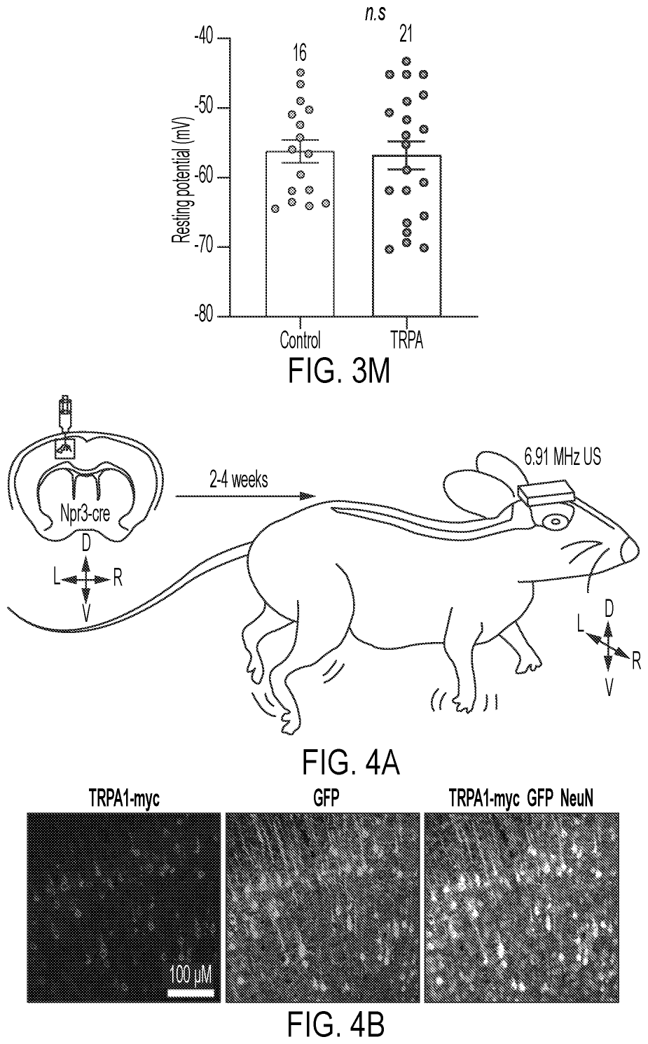

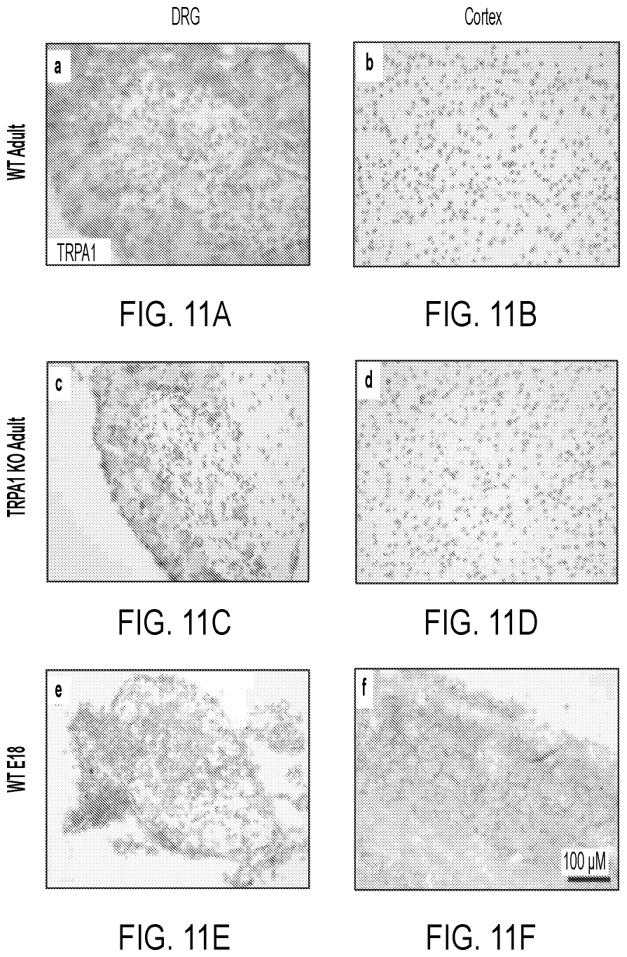

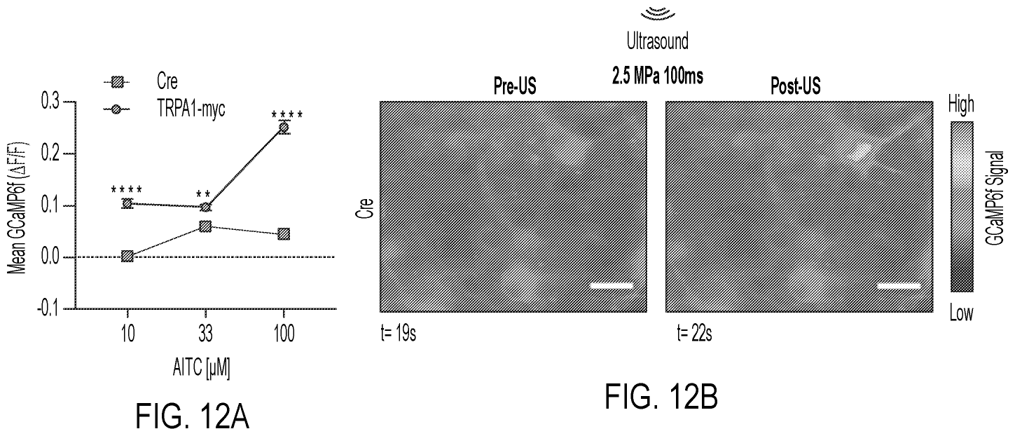

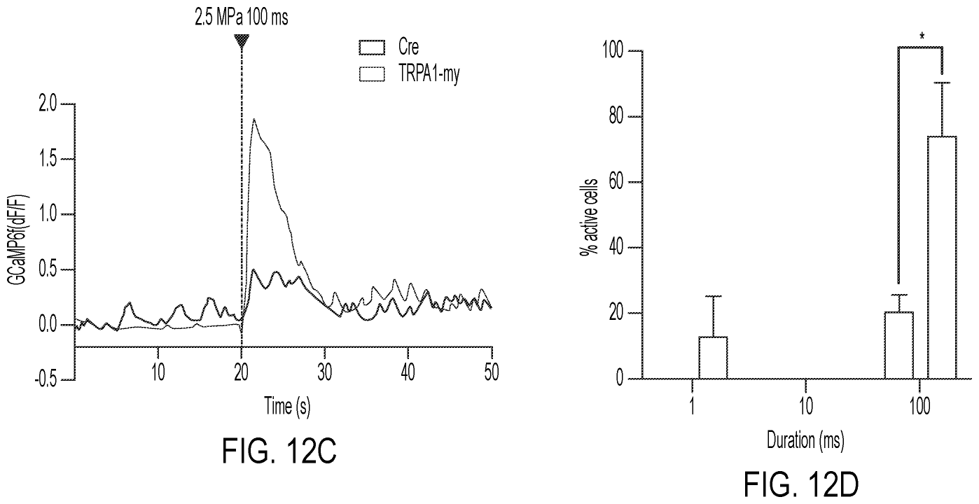

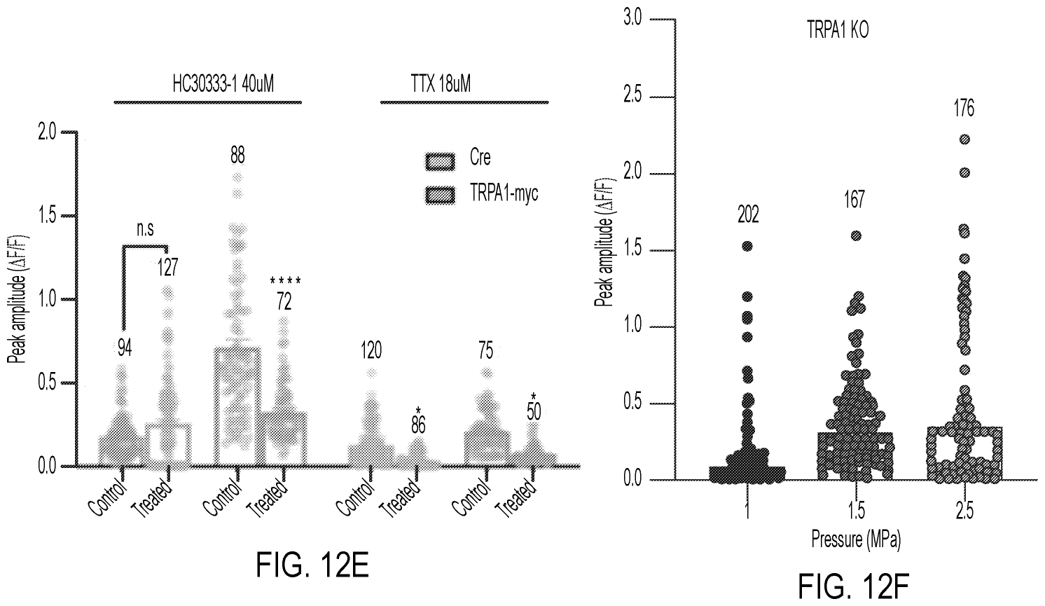

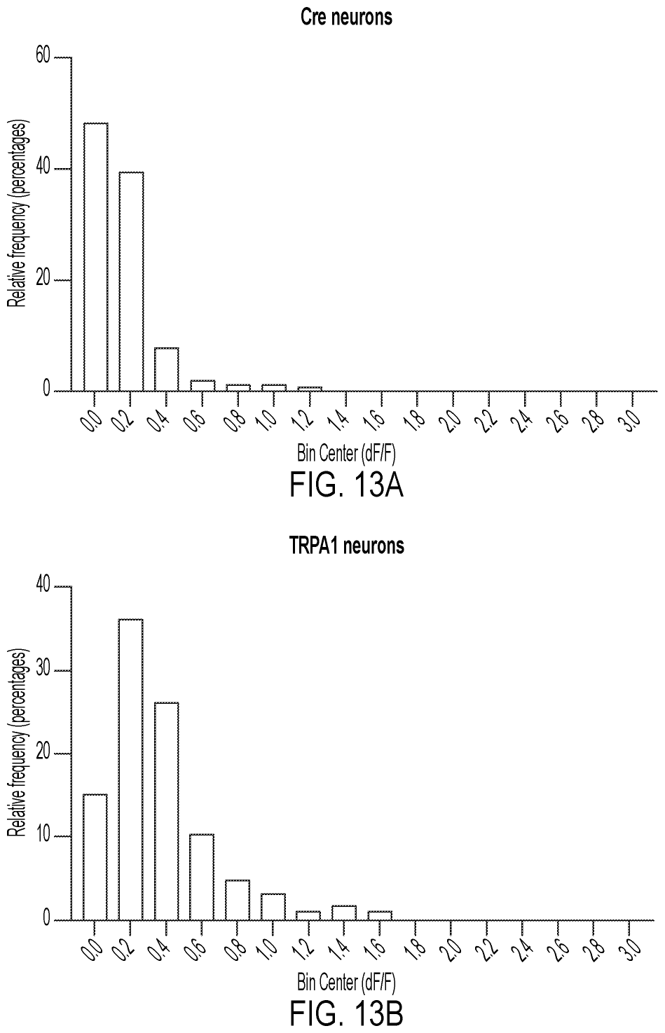

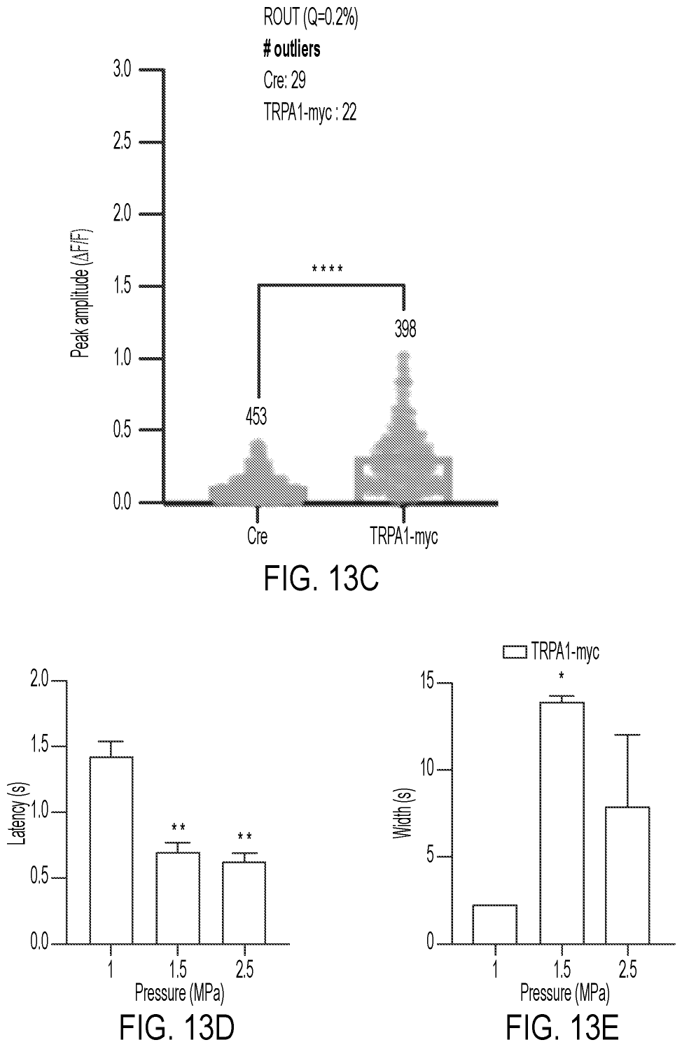

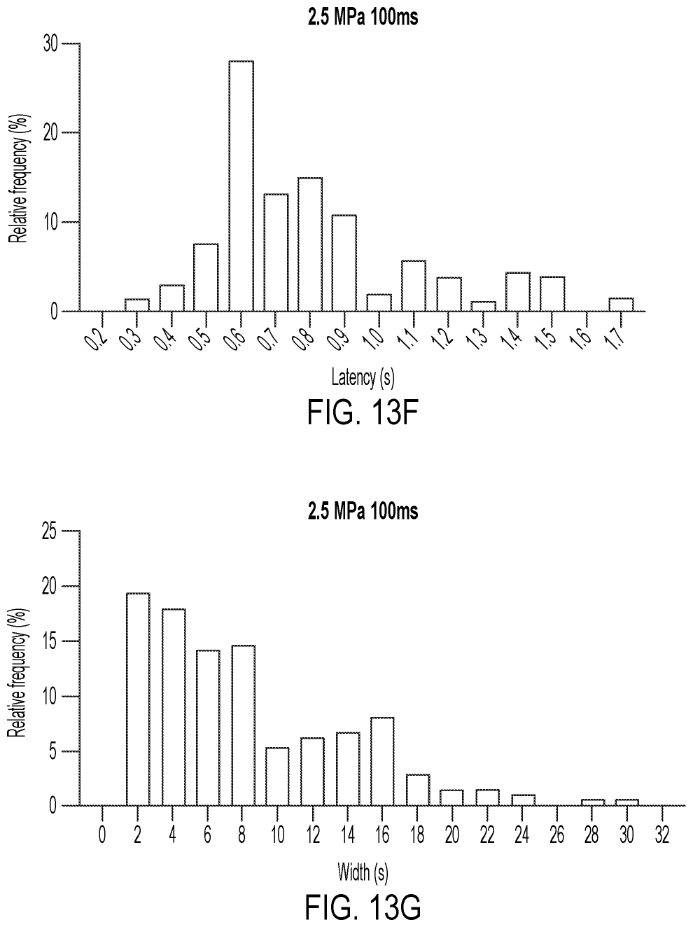

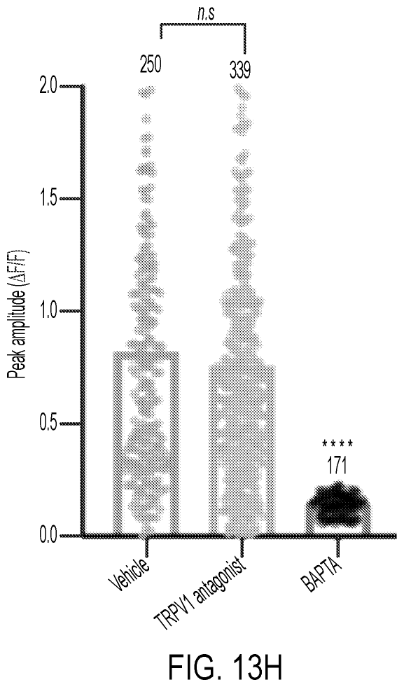

To test whether hsTRPA1 can also render neurons sensitive to ultrasound stimuli, embryonic day 18 (E18) mouse primary cortical neurons were infected with adeno-associated viral (AAV) vectors expressing either CRE-dependent hsTRPA1 or CRE-only control along with a genetically encoded calcium indicator, GCaMP6f (FIG. 3A). In this study, hsTRPA1 RNA was not detected in dorsal root ganglia (DRG) or in brains from E18 mouse (FIGS. 11A-11F/11E, 11F), consistent with previous studies. Functional expression of hsTRPA1 in infected neurons was then confirmed by monitoring their responses to a chemical agonist, AITC. It was observed that Cre-only control neurons did not respond to AITC (FIG. 12A). Consistent with the HEK cell results described herein, it was found that ultrasound triggered a significant increase in intracellular calcium in hsTRPA1-expressing neurons, but not in control CRE-expressing neurons (FIGS. 3B and 3C). Cre-expressing neurons showed some calcium responses to ultrasound, but these were significantly lower in magnitude than those observed in hsTRPA1-expressing neurons (FIGS. 12B and 12C and FIGS. 13A-13C). Both hsTRPA1 and control CRE-expressing neurons showed increased responses to longer (FIG. 12D) and more intense ultrasound stimuli (FIG. 3D). However, hsTRPA1-expressing neurons showed greater sensitivity and reduced response latency to ultrasound stimuli (FIGS. 13D and 13E), confirming that hsTRPA1 expression mediates responses to ultrasound. The majority of hsTRPA1-expressing neurons had a response latency within 500-900 ms of stimulus onset, while response durations ranged from 2-30 s (FIGS. 13F and 13G). Moreover, hsTRPA1-expressing neurons could be stimulated repeatedly without apparent deleterious effects on cell health or a substantial decrement in calcium flux (FIG. 3E), with cells returning to baseline after stimulation.

Next, studies were conducted to confirm whether ultrasound-mediated effects in control and hsTRPA1-expressing neurons were due to the TRPA1 channel. Treating hsTRPA1 expressing neurons with a TRPA1 antagonist, HC-030331, significantly attenuated their responses to ultrasound without affecting the ultrasound-evoked activity in control neurons (FIG. 12E). Furthermore, cortical neurons cultured from TRPA1−/− mice also responded to ultrasound (FIG. 12F), indicating that even undetectable levels of TRPA1 in neurons or astrocytes likely do not account for ultrasound responses in control neurons. Moreover, the sodium channel blocker, tetrodotoxin, partially blocked ultrasound responses in hsTRPA1 neurons, while completely abolishing responses in control neurons (FIG. 12E). As described herein, it was also found that sequestering extracellular calcium with BAPTA blocked neuronal responses to ultrasound (FIG. 13H). However, treating neurons with a TRPV1 antagonist had no effect on their ultrasound responses (FIG. 13H), thereby ruling out the TRPV1 heat-responsive channel's contribution to ultrasound sensitivity in TRPA1-expressing neurons either directly or through a synergistic interaction. These results show that ultrasound can directly activate AAV9-hsTRPA1 transduced neurons, leading to intracellular calcium influx, which may be amplified by voltage-gated sodium channels. In contrast, ultrasound responses in control neurons are due to a TRPA1-independent mechanism.

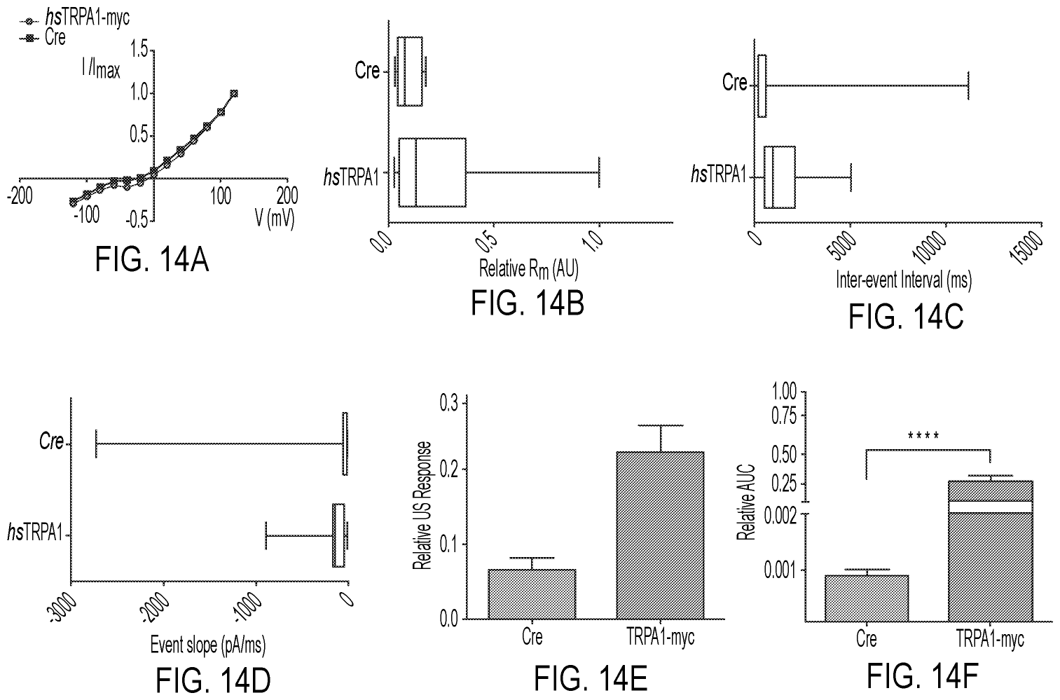

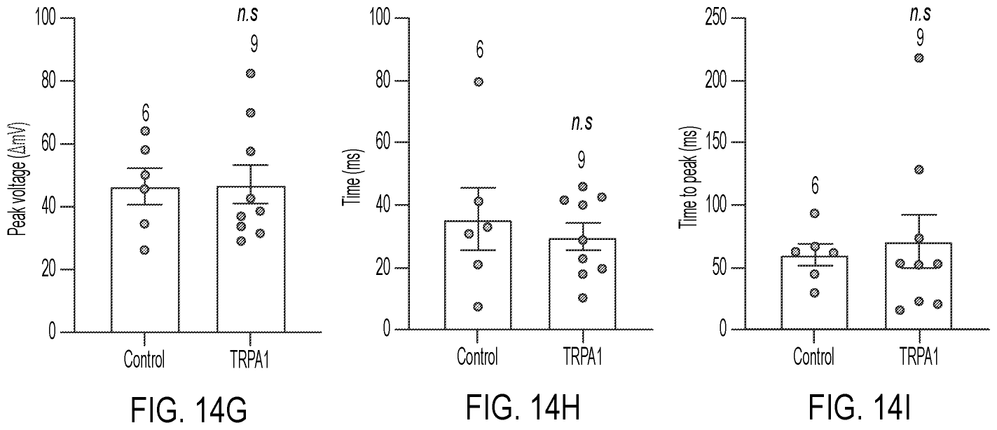

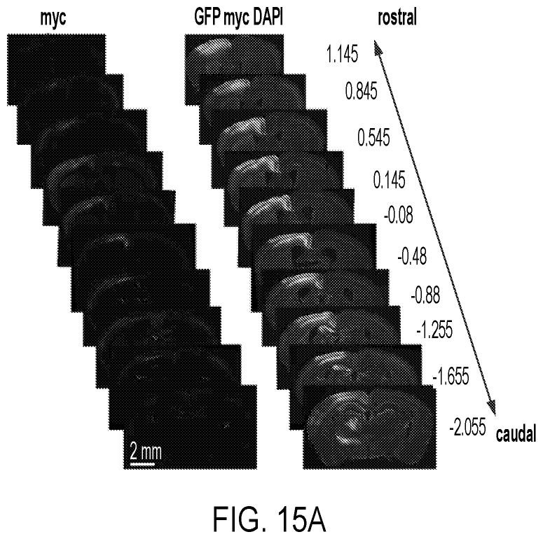

Experiments using electrophysiological methods were next performed to confirm the role of hsTRPA1 in mediating ultrasound-evoked neuronal responses. Stable membrane resistances and reliable measurements were obtained during ultrasound stimulation trials using the whole-cell patch clamp configuration (FIG. 3F). However, only responsiveness to pressures below 0.5 MPa was able to be assayed in order to ensure integrity of the patch. Similar to the HEK cell experiments described herein, it was found that AAV9-hsTRPA1 expression in transduced neurons did not alter neuronal membrane properties (FIG. 14A). In voltage clamp, Cre-expressing control neurons showed inward currents in response to ultrasound, consistent with previous studies. However, hsTRPA1-expressing neurons showed larger current responses (>400 pA) compared to controls within a few milliseconds of ultrasound stimulation (FIGS. 3G, 3H, 3I). Taken together, hsTRPA1-expressing neurons had enhanced responses to ultrasound relative to control neurons as assessed by their relative response, magnitude of peak responses, and area under the curve (AUC) metrics (FIGS. 14B-14F).

In addition, responsiveness to ultrasound was assessed in current clamp mode to evaluate action potential generation. Ultrasound stimulation triggered action potentials in neurons expressing hsTRPA1 (FIG. 3J) at pressures as low as 0.2 MPa. In contrast, control neurons showed subthreshold changes in membrane voltage that were insufficient to trigger action potentials during a majority of stimulation trials (FIG. 3K). Collectively, all assayed hsTRPA1-expressing neurons showed ultrasound-evoked action potentials, although not every ultrasound stimulus triggered an action potential, while control neurons had infrequent action potentials in response to ultrasound (FIG. 3L). Neither the latency, peak voltage, or time to peak of action potentials in response to ultrasound were altered by the expression of TRPA1 (FIGS. 14G-14I), and the membrane resting potential was similar between the two groups (FIG. 3M). The results demonstrate that ultrasound triggers increased currents and action potentials in hsTRPA1-expressing neurons even at ultrasound pressures well below those shown to elicit responses by calcium imaging.

### Example 4: hsTRPA1 Confers Ultrasound Sensitivity In Vivo

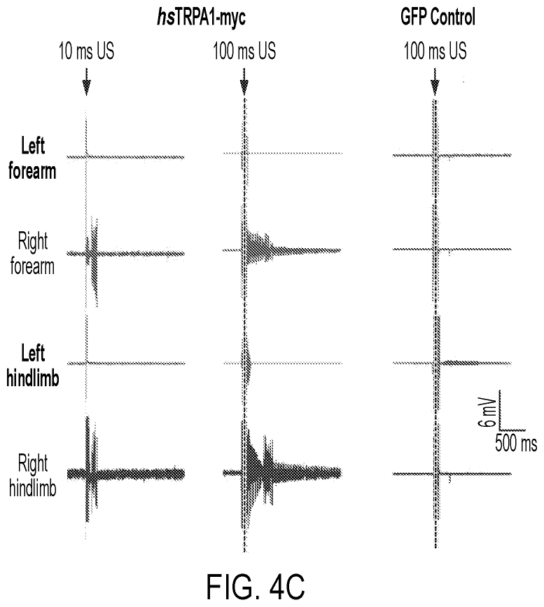

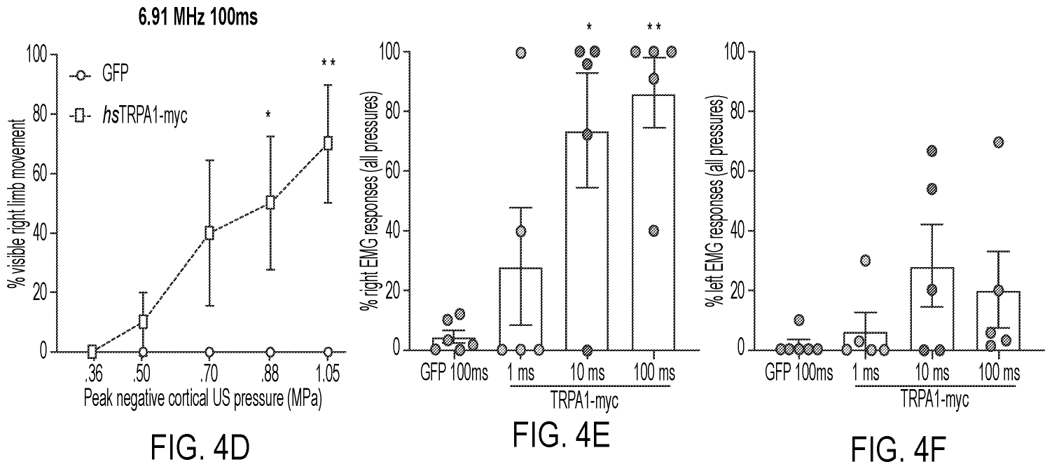

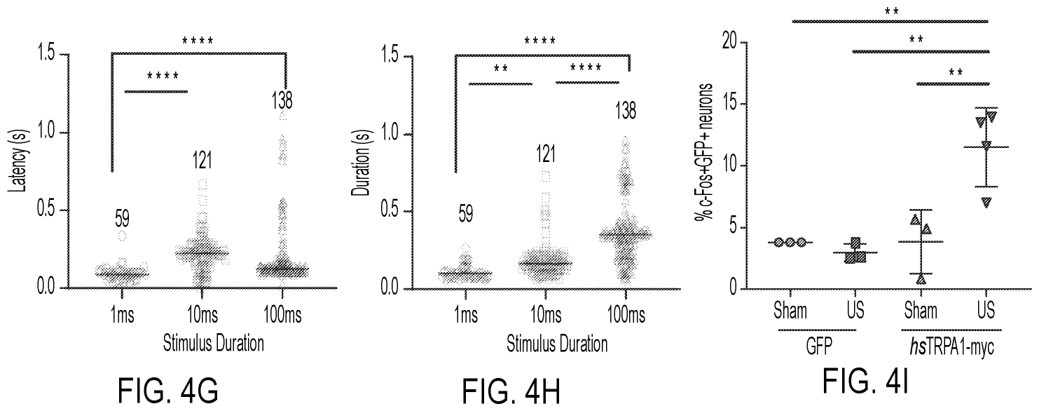

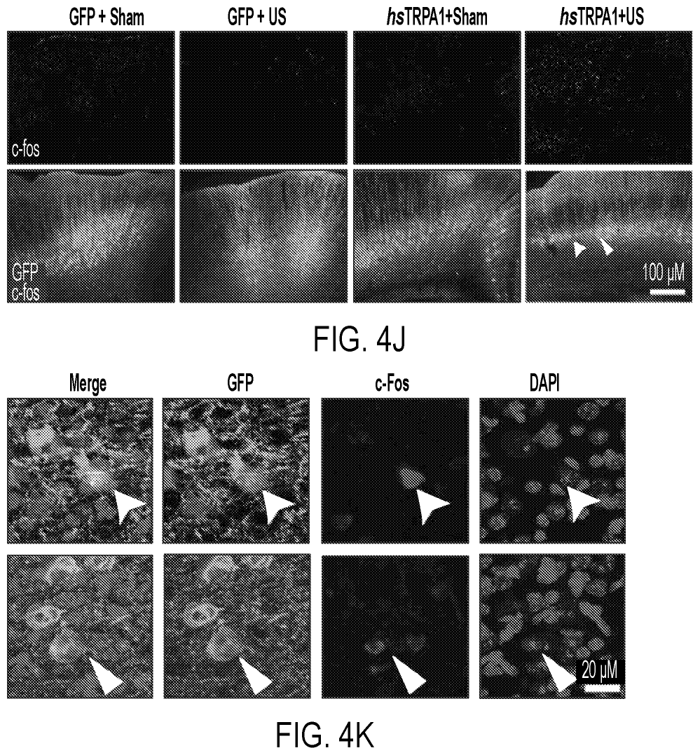

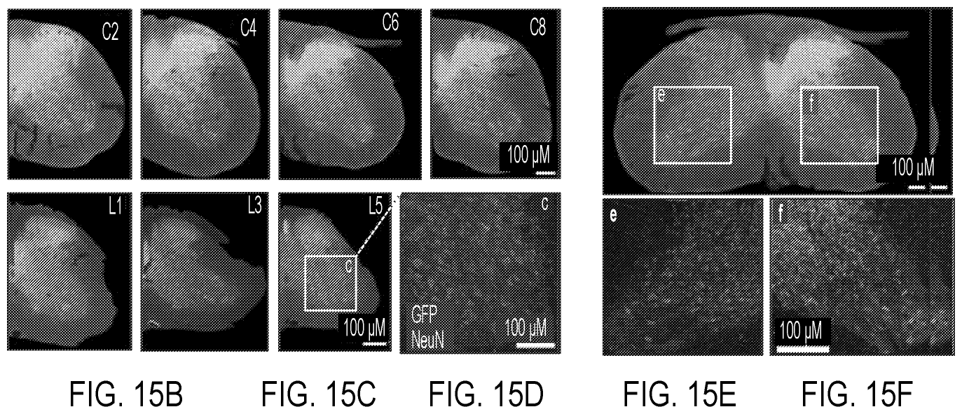

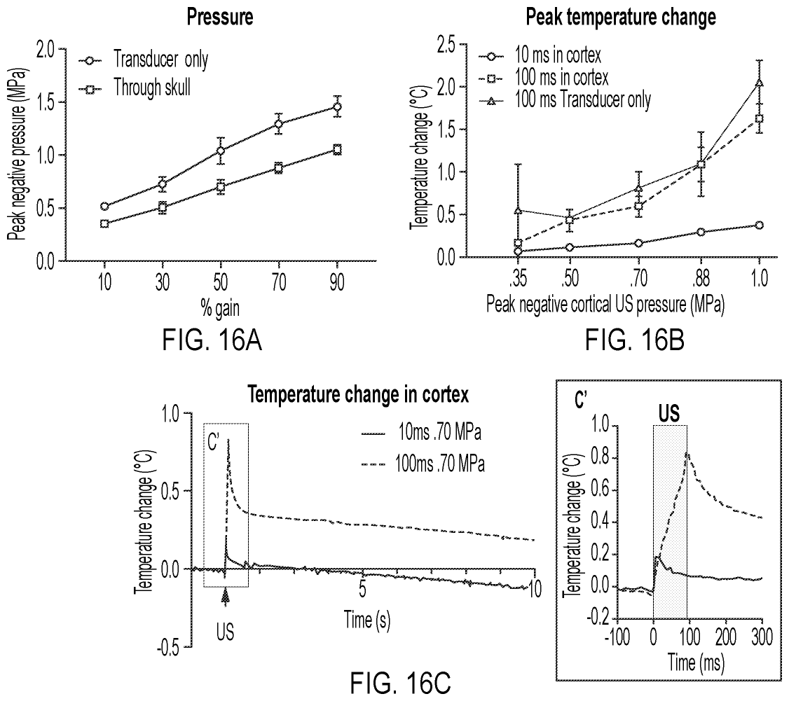

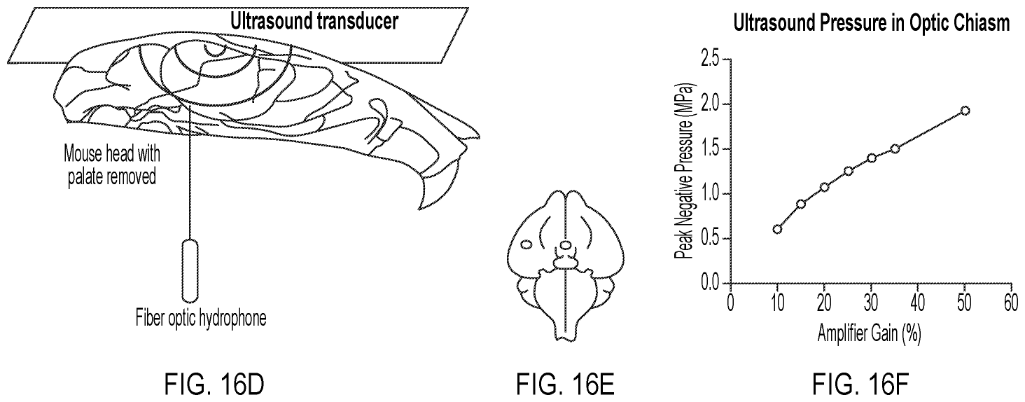

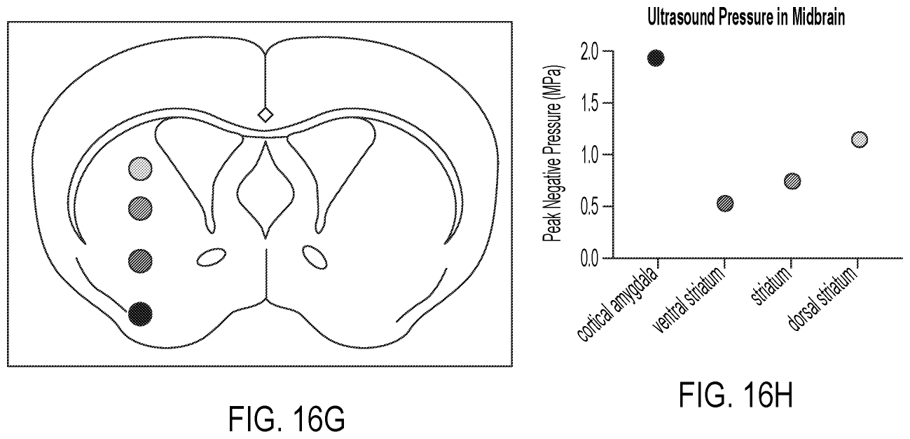

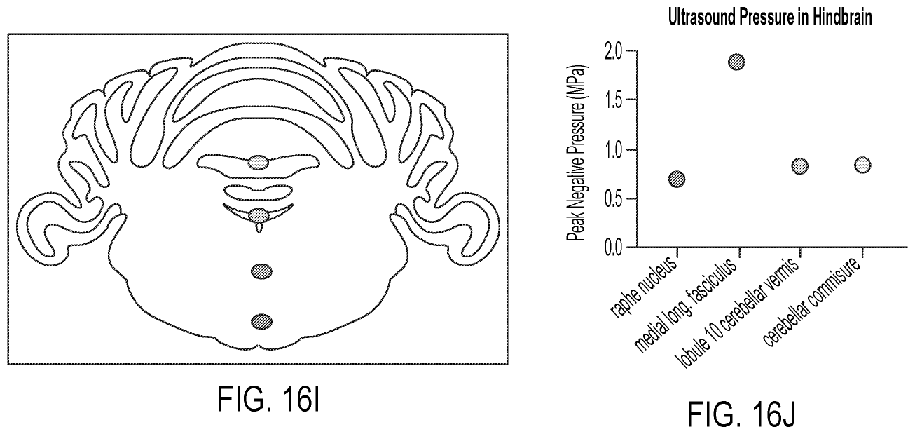

Studies were conducted as described herein to determine whether hsTRPA1 can be used as a sonogenetic tool for temporally-selective activation of neurons in vivo. To this end, CRE-dependent AAV was used to restrict the expression of hsTRPA1 to layer V motor cortical neurons in Npr-3 CRE transgenic mice (Daigle, T. L. et al., Cell 174, 465-480 e422, doi:10.1016/j.cell.2018.06.035 (2018)), (FIG. 4A). In situ hybridization was first used to confirm that cortical neurons do not express endogenous TRPA1 (FIGS. 11A-11F). Consistently, data from Allen Brain Atlas, Biogps and Brain-seq projects confirm that TRPA1 expression is undetectable in the brain (Sunkin, S. M. et al., Nucleic Acids Research 41, D996-D1008, doi:10.1093/nar/gks1042 (2012); Wu, C. et al., Genome Biol 10, R130, doi:10.1186/gb-2009-10-11-r130 (2009); Zhang, Y. et al., J Neurosci 34, 11929-11947, doi:10.1523/JNEUROSCI.1860-14.2014 (2014)). Using coordinates based on a previous study (Ueno, M. et al., Cell reports 23, 1286-1300 e1287, doi:10.1016/j.celrep.2018.03.137 (2018), AAV9 encoding myc-tagged hsTRPA1 were co-injected with AAV9 encoding GFP into the left motor cortex to visualize the transfected neurons. This approach robustly transduced layer V cortical neurons throughout both the forelimb and hindlimb motor cortices (FIG. 4B and FIG. 15A) and and their projections in the right cervical and lumbar spinal cord (FIG. 15B) Using the 6.91 MHz lithium niobate transducer coupled to the exposed skull through ultrasound gel, the ability to deliver ultrasound to cortical regions was verified. This in vivo approach delivered peak negative pressures ranging from 0.35-1.05 MPa with minimal temperature changes to cortex and other brain regions (FIGS. 16A-16J). The results showed that deeper brain regions were able to be targeted non-invasively with the ultrasound delivery system described herein.

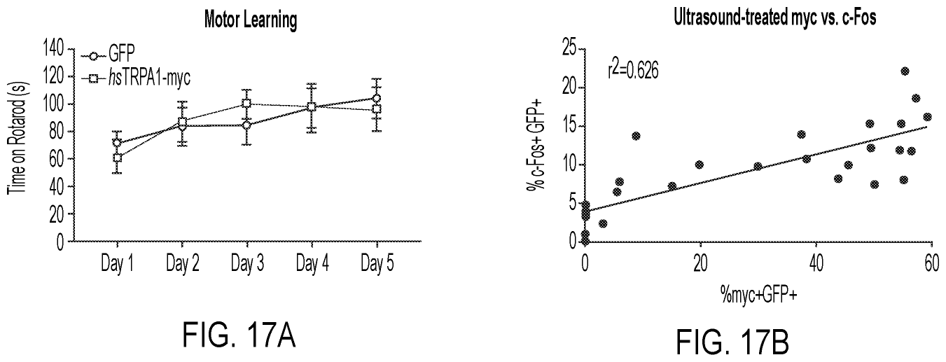

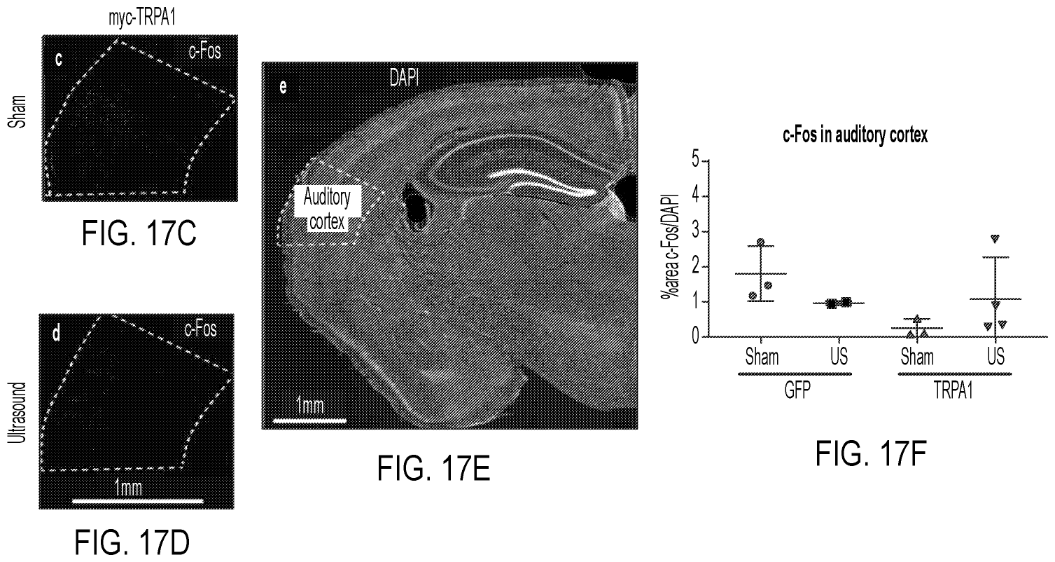

After 2-4 weeks following intracranial injection, ultrasound-evoked electromyography (EMG) responses were monitored in the bilateral biceps brachii and biceps femorii muscles. Ultrasound evoked few EMG responses and no visible movements in any of the limbs of the GFP-control mice (FIGS. 4D, 4E, 4F). In contrast, animals injected with AAV9-hsTRPA1 in the left motor cortex showed dose-dependent EMG responses and visible movement in their right fore- and/or hindlimbs (FIGS. 4D and 4E). EMG responses in the left forelimb occurred infrequently, suggesting circuit specific sensitivity to ultrasound (FIG. 4F). Consistently, some of the transduced cortical neuron processes innervating the left forelimb motor pools were observed (FIG. 15D-15F). Moreover, while most EMG responses occurred within 1 second of ultrasound stimulation, the latency and duration of these responses increased with stimulus duration (FIGS. 4G and 4H). To confirm functional activation of cortical neurons, it was tested whether ultrasound stimulation activated c-fos expression in motor cortical neurons expressing hsTRPA1. While ultrasound stimulation had no effect on the number of c-fos positive cells in animals expressing GFP, it significantly increased the number of c-fos positive cells in cortical motor neurons of hsTRPA1-expressing transgenic mice (FIGS. 4I, 4J and 4K and FIG. 17B). This upregulation was specific to the cortex and increased c-fos expression was not detected in the auditory cortex in these animals (FIGS. 17C-17F), suggesting that the ultrasound-mediated effect does not involve incidental activation of the auditory cortex as has been previously suggested.

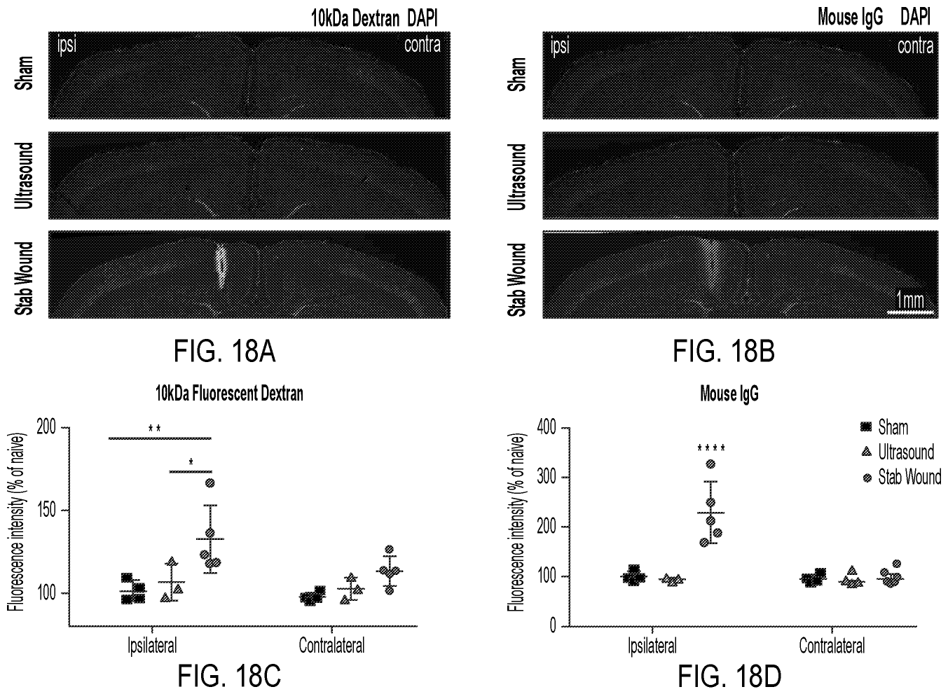

As described herein, both in vitro and in vivo neural circuits could be reliably activated using sonogenetics. To assess safety in-vivo, two metrics of safety, namely, the effect of cortical TRPA1 expression on a motor learning task and the effect of sustained ultrasound delivery on integrity of the blood-brain barrier, were assessed. It was found that both hsTRPA1- and GFP-expressing animals had comparable ability to learn the rotarod task (FIG. 17A). Similarly, it was found that animals receiving 1 hour of intermittent ultrasound stimulation had no damage to their blood brain barrier. In contrast to stab wound positive control animals in which both 10 kDa fluorescent dextran accumulated and mouse IgG showed elevated binding, no increase in fluorescence was observed in animals receiving ultrasound, indicating that neither large nor small proteins were able to leak through the blood brain barrier during sonication (FIGS. 18A-18D). Taken together, these results showed that ultrasound can be used to selectively modulate neurons infected with AAV9-hsTRPA1 through an intact mouse skull at a frequency and pressure that neither affects normal behavior nor causes blood-brain barrier impairment.

The studies and results described herein demonstrate that hsTRPA1 is a candidate sonogenetic protein that confers ultrasound sensitivity to mammalian HEK cells and rodent neurons in vitro and in vivo. Using an unbiased screen, hsTRPA1-expressing HEK cells were found to show ultrasound-evoked calcium influx and membrane currents. Moreover, critical components of hsTRPA1 ultrasound sensitivity were revealed, including the N-terminal tip region and interactions with the actin cytoskeleton and cholesterol. It was also shown that hsTRPA1 potentiated ultrasound-evoked calcium transients and enabled ultrasound-evoked action potentials in rodent primary neurons. The studies described herein provide the first report of ultrasound-induced action potentials using patch clamp at clinically relevant frequencies, lower than 25 MHz. In addition, hsTRPA1 was used to selectively activate neurons within an intact mouse skull using single pulses of ultrasound ranging from 1-100 msecs. These ultrasound parameters are below the range associated with cavitation effects. Accordingly, no damage to the blood-brain barrier was observed, even with intermittent ultrasound delivered over 60 minutes. Moreover, overexpressing hsTRPA1 did not cause behavioural changes on rotarod assays, confirming the viability of this candidate for sonogenetics use across species. The results obtained in the studies described herein are in contrast to a previous study showing that mouse TRPA1 functions in astrocytes and use a Best1 dependent pathway to release glutamate depolarizing neighboring neurons upon ultrasound (Oh, S. J. et al., Curr Biol 29, 3386-3401 e3388, doi:10.1016/j.cub.2019.08.021 (2019). The model of Oh et al. requires TRPA1 and Best1 expression in astrocytes, which is highly controversial. In addition, the results described herein confirm that TRPA1 is undetectable in the brain and that ultrasound can non-invasively activate neurons that express exogenous hsTRPA1 in vitro and in vivo, thus indicating the viability of the methods as described herein.

Ultrasound has been shown to have neuromodulatory effects in mice, non-human primates, and even human subjects, although the underlying mechanisms remain poorly understood. Overexpressing the mechanosensory receptor TRP-4 (a TRP-N homolog) in C. elegans neurons was previously shown to render them sensitive to short pulses of ultrasound, identifying the first putative sonogenetic candidate. Multiple groups have since identified additional ultrasound-sensitive candidates, including MscL, Prestin, Piezo, TREK, MEC-4, TRPC1, TRPP2 and TRPM4 using in vitro assays. Of these, a mutated form of MscL showed activity in vivo (Qiu, Z. et al., Cell reports 32, 108033, doi:https://doi.org/10.1016/j.celrep.2020.108033 (2020)). Notwithstanding, there exist both value and a need to identify new sonogenetic candidates to extend the toolset for use in mammals and humans, and to develop channels that respond to different ranges of frequency and pressure. As described herein, hsTRPA1 and its mammalian homologs were identified as top hits for high frequency sonogenetic candidates in an unbiased screen from a curated library of mechanosensory proteins, emphasizing the unique nature of this protein.

Previous studies have shown that ankyrin repeats form a super helical coil that could act as a spring mechanism for mechanosensitive gating in NOMPC/TRPN1 (Sotomayor, M. et al., Structure 13, 669-682, doi:10.1016/j.str.2005.03.001 (2005)). As described and demonstrated herein, the TRPA1 N-terminal tip domain, particularly the first 25 amino acids, may be critical for ultrasound sensitivity and is highly similar in mammalian TRPA1 variants that showed sensitivity to ultrasound, but varies across non-mammalian chordate TRPA1 homologs that were not ultrasound sensitive. Furthermore, a chimera composed of the amTRPA1 N-terminal tip on hsTRPA1 also lacks responses to ultrasound, providing support that this region is important for tuning ultrasound sensitivity in mammalian TRPA1 variants. It has been reported that variations in the N-terminal tip of TRPA1 affect its temperature sensitivity, suggesting that this region of TRPA1 can regulate channel function (Kang, K. et al., Nature, 481, 76-80, doi:10.1038/nature10715 (2011)). As also shown herein, an intact actin cytoskeleton is required for hsTRPA1 ultrasound responses. Consistently, it has been reported that the actin cytoskeleton can either directly interact with mechanosensitive channels or interact with the plasma membrane to modify mechanosensation.

Based on the studies and results described herein, and without wishing to be bound by theory, the hsTRPA1 N-terminal tip region may interact with the actin cytoskeleton to transduce ultrasound-induced membrane perturbations into changes in intracellular calcium. Analysis of TRPA1 sequences across homologs described herein further suggested that a CRAC domain that is thought to mediate interactions with cholesterol is heavily modified or missing from the second transmembrane domain of ultrasound-insensitive variants. Indeed, interaction with the lipid bilayer was found to be critical for ultrasound sensitivity of hsTRPA1, as treating hsTRPA1-expressing cells with MCD, which removes cholesterol, attenuated their responses to ultrasound, but not to a chemical agonist. Furthermore, mutation of the central tyrosine that is critical for cholesterol interaction of the CRAC domain likewise impaired ultrasound-sensitivity, but not responses to AITC. These results as described herein are consistent with reports of others showing that TRPA1 activation requires membrane lipid interactions.

Previous studies have shown that naïve neurons can respond to ultrasound, both in vitro and in vivo. Similarly, as described herein, it was found that the ultrasound parameters used in the studies described herein can also use can also trigger increased currents and intracellular calcium in naïve neurons in vitro. However, these responses are significantly smaller than those observed in hsTRPA1-expressing neurons, and ultrasound-evoked action potentials were rarely detected at the frequency and pressures tested. Additionally, responses in control neurons in vitro may be an artefact of the 2-dimensional cultures, interactions with the substrate, or interactions with the patch pipette in electrophysiology. Experiments using more physiologically relevant systems, such as brain slices and 3-dimensional neuronal cultures, can be conducted to determine the extent of the endogenous neuronal response to ultrasound at 6.91 MHz. Moreover, as described herein, it was found that ultrasound responses in control neurons were unlikely to be TRPA1-mediated, as these are not reduced upon treatment with TRPA1 antagonists, and because neurons cultured from TRPA1−/− mice also responded to ultrasound. Accordingly, endogenous TRPA1 was not detected in E18 brain tissue. Together, these results suggest that intrinsic neuronal responses to the ultrasound parameters described herein are unlikely to involve TRPA1 in neurons. Instead, a recent study found that knocking down TRPP1, TRPP2, Piezo, TRPC1 and TRPM4 each partially reduced ultrasound-evoked neuronal responses (Yoo, S., et al., bioRxiv (2020). As also demonstrated herein, blocking voltage-gated sodium channels eliminated neuronal ultrasound-evoked calcium responses. Therefore, intrinsic ultrasound neuromodulation may involve a number of mechanosensitive channels whose activity is further amplified by voltage-gated sodium channels. As described herein, hsTRPA1-expressing neurons were shown to maintain partial ultrasound sensitivity in the presence of a sodium-channel blocker, confirming that hsTRPA1-mediated ultrasound sensitivity is at least partially independent from the mechanism contributing to ultrasound activation in control neurons.

Finally, the studies and results described herein demonstrate that hsTRPA1 can be used to selectively activate a specific cell population in vivo with ultrasound pulses (1-100 msecs) from a 6.91 MHz transducer. The results described herein suggest that ultrasound might not act as a simple stretch force on the membrane and indicate that channels that likewise sense other perturbations, including lipid bilayer changes, may be good candidates for sonogenetics. Moreover, the identification of specific interactions (namely, actin and membrane cholesterol) and the N-terminal tip domain in hsTRPA1 as described herein allow for rapidly engineering this channel for enhanced ultrasound sensitivity and ion permeability. Advantageously, based on the studies and results described herein, hsTRPA1 and its variants could be used to non-invasively control neurons and other cell types across species.

### Example 5: TRPA1 (Clone 63) Expression Renders Neurons Responsive to Ultrasound Stimulation

Ultrasound is non-invasive and can cause a small amount of mechanical deformation in the focal zone. As described supra, non-native (non-naturally occurring) mechanosensitive channel proteins that respond to ultrasound-triggered mechanical deformation were identified, thus allowing for manipulation of specific cells involving the use of ultrasound. Clone 63 represents a channel protein that is not found in nature, is responsive to ultrasound stimulation, and is effective sonogenetically both in cell culture (in vitro) and in animals (in vivo).

To generate ultrasound responsive channel proteins, an expression screen was performed in HEK 293 cells (HEK cells). Individual candidate proteins were expressed in the HEK cells along with a calcium sensor and their sensitivity to ultrasound was assessed. If the candidate protein was ultrasound-sensitive, the cells would respond to ultrasound upon ultrasound stimulation. The screen included about 200 mechanosensitive proteins.

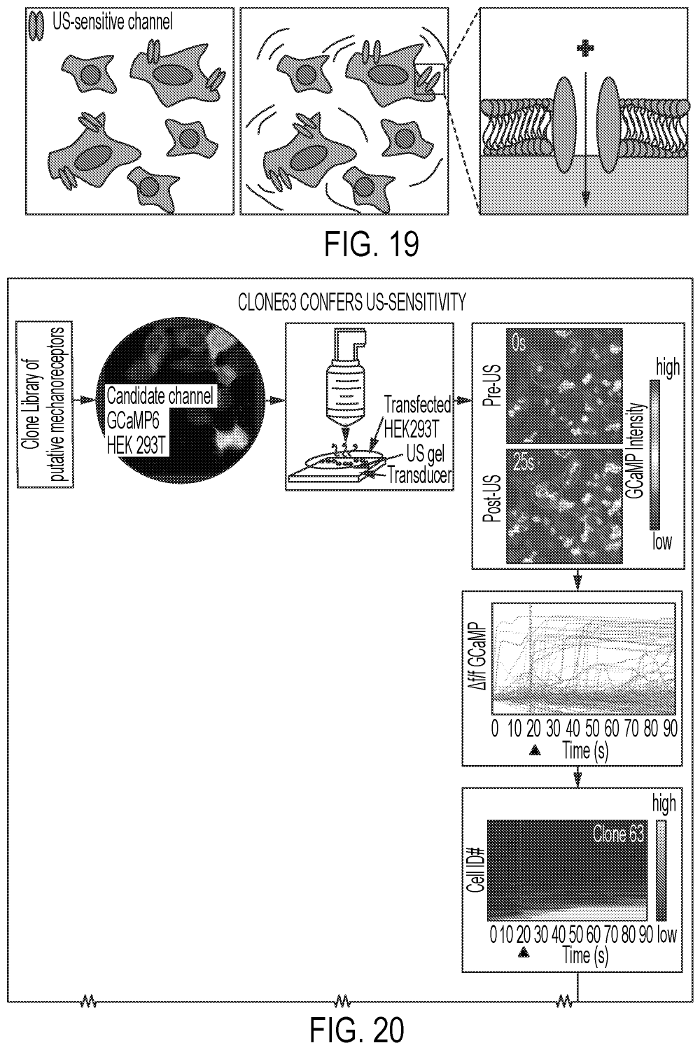

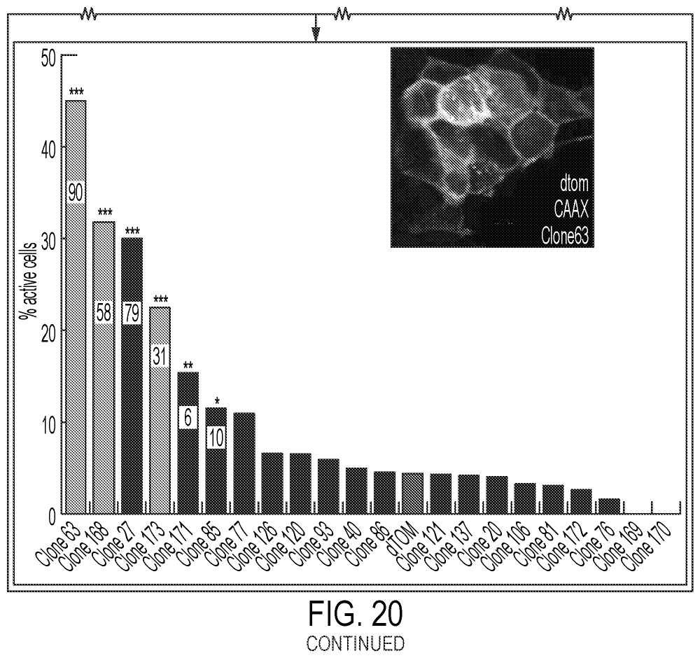

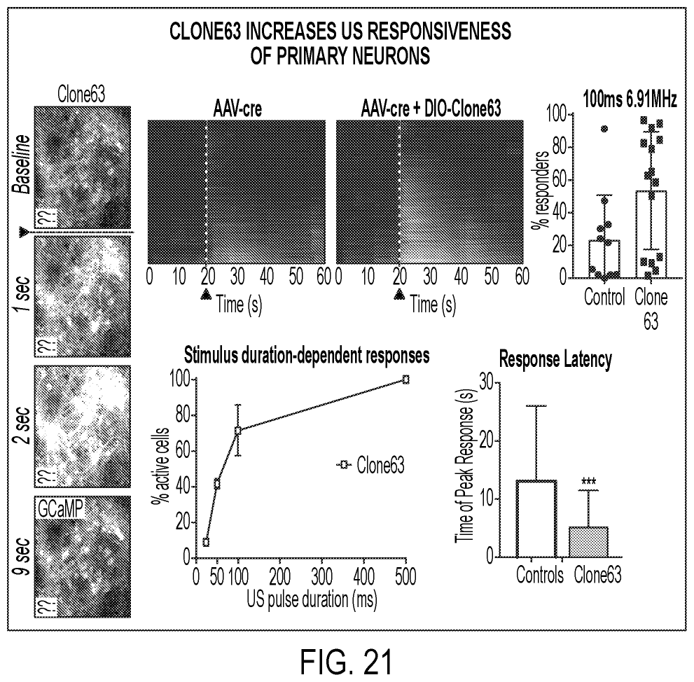

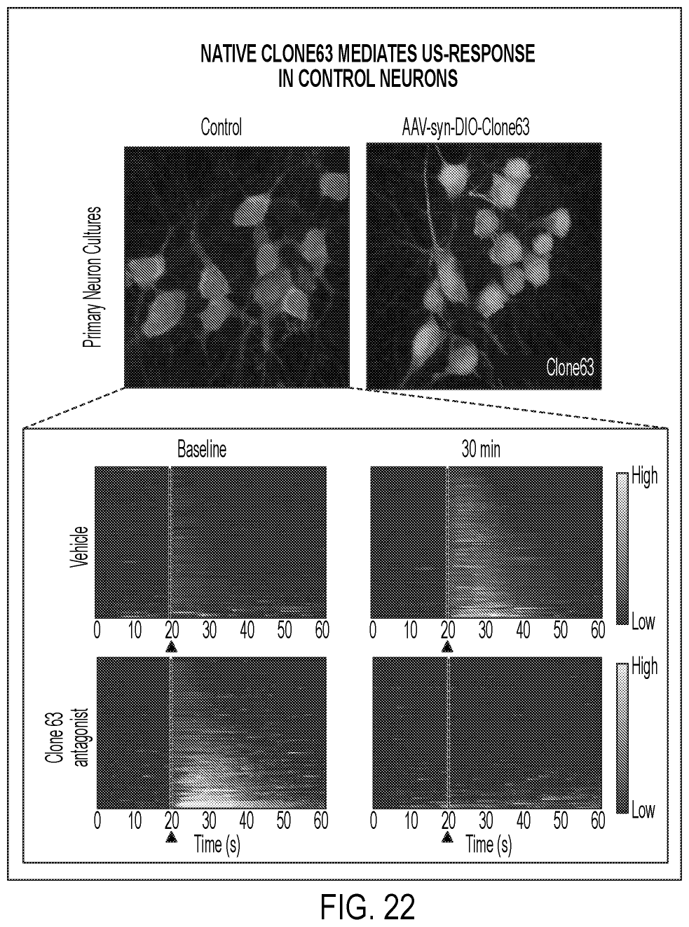

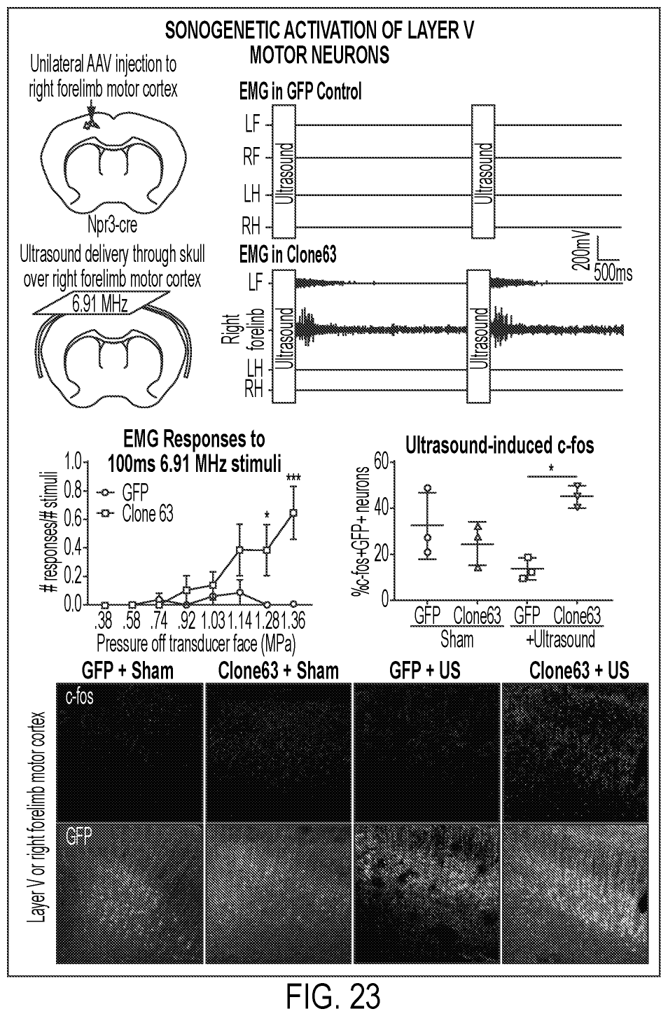

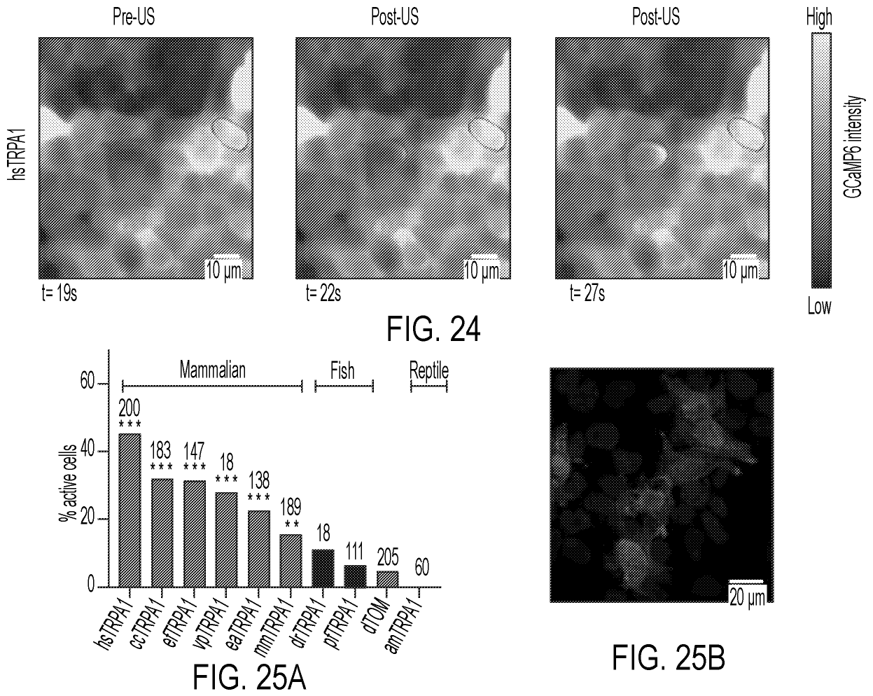

The human transient receptor potential A (hsTRPA1) channel protein was identified as an ultrasound sensitive channel protein (FIG. 24). Clone 63 refers to a human TRPA1 channel protein that was expressed on the membrane of human cells. FIGS. 19-23 show that cells transduced with a viral vector encoding human TRPA1 (e.g., Clone 63) were responsive to ultrasound stimulation.

TRPA channels from different species were assayed for their responsiveness to ultrasound stimulation/activation, and it was found that the human TRPA1 mechanosensitive protein (e.g., Clone 63), which was expressed on the cell membrane, was the most responsive to ultrasound stimulation. (FIGS. 25A and 25B).

### Example 6: Human TRPA1 (Clone 63) and Variant Channel Proteins

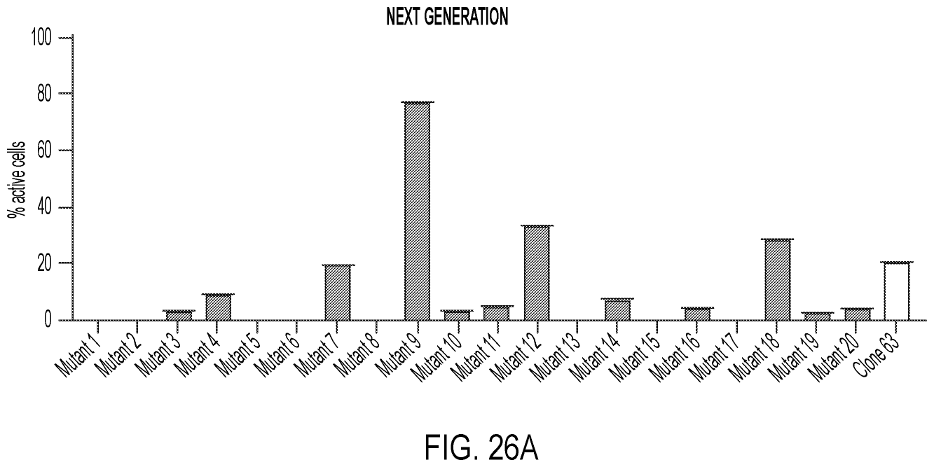

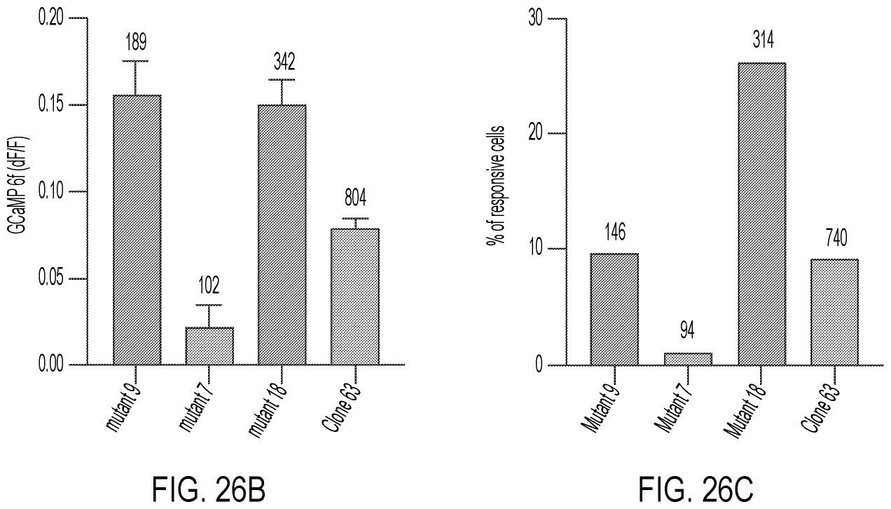

Clone-63 is a human TRPA1 channel protein that is expressed in human cells, namely, on the cell membrane. Variant (mutant) proteins were generated and tested them in the ultrasound activation assay (FIGS. 26A and 26B).

The amino acid sequence of the human TRPA1 channel protein Clone 63 (non-mutated/WT) is presented below:

WT Clone 63
(SEQ ID NO: 4)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDFNVSIHSKSKDKK
SPLHFAASYGRINTCQRLLQDISDTRLLNEGDLHG
MTPLHLAAKNGHDKVVQLLLKKGALFLSDHNGWTA
LHHASMGGYTQTMKVILDTNLKCTDRLDEDGNTAL
HFAAREGHAKAVALLLSHNADIVLNKQQASFLHLA
LHNKRKEVVLTIIRSKRWDECLKIFSHNSPGNKCP
ITEMIEYLPECMKVLLDFCMLHSTEDKSCRDYYIE
YNFKYLQCPLEFTKKTPTQDVIYEPLTALNAMVQN
NRIELLNHPVCKEYLLMKWLAYGFRAHMMNLGSYC
LGLIPMTILVVNIKPGMAFNSTGIINETSDHSEIL
DTTNSYLIKTCMILVFLSSIFGYCKEAGQIFQQKR
NYFMDISNVLEWIIYTTGIIFVLPLFVEIPAHLOW
QCGAIAVYFYWMNFLLYLQRFENCGIFIVMLEVIL
KTLLRSTVVFIFLLLAFGLSFYILLNLQDPFSSPL
LSIIQTFSMMLGDINYRESFLEPYLRNELAHPVLS
FAQLVSFTIFVPIVLMNLLIGLAVGDIAEVQKHAS
LKRIAMQVELHTSLEKKLPLWFLRKVDQKSTIVYP
NKPRSGGMLFHIFCFLFCTGEIRQEIPNADKSLEM
EILKQKYRLKDLTFLLEKQHELIKLIIQKMEIISE
TEDDDSHCSFQDRFKKEQMEQRNSRWNTVLRAVKA
KTHHLEP

In an embodiment, an amino acid sequence having at least 85%, at least 88%, at least 90%, at least 91%, at least 92%, at least 93%, at least 94%, at least 95%, at least 96%, at least 97%, at least 98%, at least 99%, or 100% identity to the amino acid sequence of Clone 63 above is encompassed.

The amino acid sequence of a variant human TRPA1 channel protein (Mutant 18) is presented below:

Clone 63-Mutant 18
(SEQ ID NO: 5)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQQ
PYGLKNLRPEFMQMQQIKELVMDEDNDGCTPLHYA
CRQGGPGSVNNLLGFNVSIHSKSKDKKSPLHFAAS
YGRINTCQRLLQDISDTRLLNEGDLHGMTPLHLAA
KNGHDKVVQLLLKKGALFLSDHNGWTALHHASMGG
YTQTMKVILDTNLKCTDRLDEDGNTALHFAAREGH
AKAVALLLSHNADIVLNKQQASFLHLALHNKRKEV
VLTIIRSKRWDECLKIFSHNSPGNKCPITEMIEYL
PECMKVLLDFCMLHSTEDKSCRDYYIEYNFKYLQC
PLEFTKKTPTQDVIYEPLTALNAMVQNNRIELLNH
PVCKEYLLMKWLAYGFRAHMMNLGSYCLGLIPMTI
LVVNIKPGMAFNSTGIINETSDHSEILDTTNSYLI
KTCMILVFLSSIFGYCKEAGQIFQQKRNYFMDISN
VLEWIIYTTGIIFVLPLFVEIPAHLOWQCGAIAVY
FYWMNFLLYLQRFENCGIFIVMLEVILKTLLRSTV
VFIFLLLAFGLSFYILLNLQDPFSSPLLSIIQTFS
MMLGDINYRESFLHPYLRNELAHPVLSFAQLVSFT
IFVPIVLMNLLIGLAVGDIAEVQKHASLKRIAMQV
ELHTSLEKKLPLWFLRKVDQKSTIVYPNKPRSGGM
LFHIFCFLFCTGEIRQEIPNADKSLEMEILKQKYR
LKDLTFLLEKQHELIKLIIQKMEIISETEDDDSHC
SFQDRFKKEQMEQRNSRWNTVLRAVKAKTHHLEP

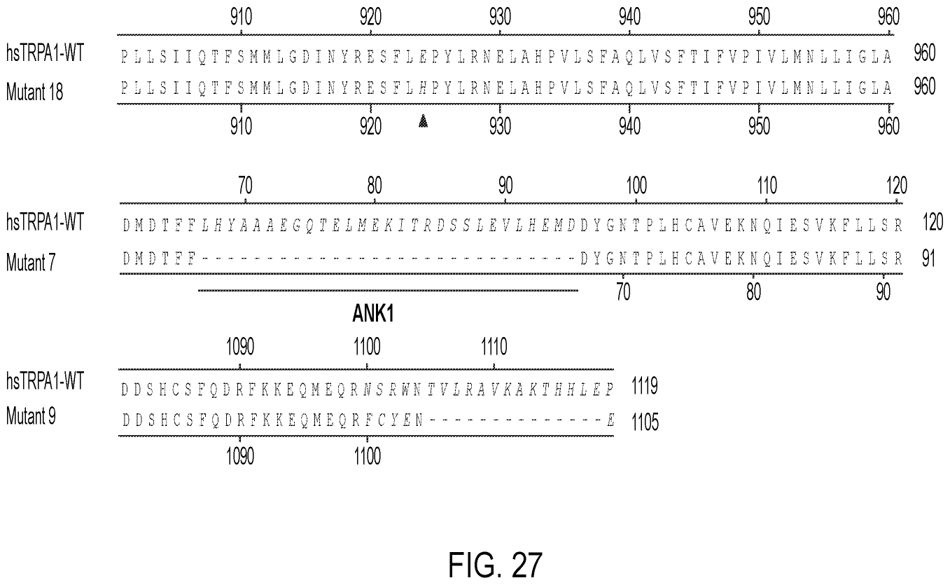

In the above amino acid sequence of Clone 63-Mutant 18, position 924 is a histidine (H) residue (bold underline in the above sequence), while this position in the hsTRPA1 WT Clone 63 amino acid sequence is a glutamate (E) residue. (FIG. 27). In an embodiment, an amino acid sequence having at least 85%, at least 88%, at least 90%, at least 91%, at least 92%, at least 93%, at least 94%, at least 95%, at least 96%, at least 97%, at least 98%, at least 99%, or 100% identity to the amino acid sequence of Mutant 18 above is encompassed.

The amino acid sequence of a variant human TRPA1 channel protein (Mutant 9) is presented below:

Clone 63-Mutant 9
(SEQ ID NO: 6)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDFNVSIHSKSKDKK
SPLHFAASYGRINTCQRLLQDISDTRLLNEGDLHG
MTPLHLAAKNGHDKVVQLLLKKGALFLSDHNGWTA
LHHASMGGYTQTMKVILDTNLKCTDRLDEDGNTAL
HFAAREGHAKAVALLLSHNADIVLNKQQASFLHLA
LHNKRKEVVLTIIRSKRWDECLKIFSHNSPGNKCP
ITEMIEYLPECMKVLLDFCMLHSTEDKSCRDYYIE
YNFKYLQCPLEFTKKTPTQDVIYEPLTALNAMVQN
NRIELLNHPVCKEYLLMKWLAYGFRAHMMNLGSYC
LGLIPMTILVVNIKPGMAFNSTGIINETSDHSEIL
DTTNSYLIKTCMILVFLSSIFGYCKEAGQIFQQKR
NYFMDISNVLEWIIYTTGIIFVLPLFVEIPAHLOW
QCGAIAVYFYWMNFLLYLQRFENCGIFIVMLEVIL
KTLLRSTVVFIFLLLAFGLSFYILLNLQDPFSSPL
LSIIQTFSMMLGDINYRESFLEPYLRNELAHPVLS
FAQLVSFTIFVPIVLMNLLIGLAVGDIAEVQKHAS
LKRIAMQVELHTSLEKKLPLWFLRKVDQKSTIVYP
NKPRSGGMLFHIFCFLFCTGEIRQEIPNADKSLEM
EILKQKYRLKDLTFLLEKQHELIKLIIQKMEIISE
TEDDDSHCSFQDRFKKEQMEQRFCYENE

In the above amino acid sequence of Clone 63-Mutant 9, the terminal 6 amino acid residues of the polypeptide (in bold underline), differ from the amino acid residues of the hsTRPA1 WT Clone 63 amino acid sequence (FIG. 27). In an embodiment, an amino acid sequence having at least 85%, at least 88%, at least 90%, at least 91%, at least 92%, at least 93%, at least 94%, at least 95%, at least 96%, at least 97%, at least 98%, at least 99%, or 100% identity to the amino acid sequence of Mutant 9 above is encompassed.

The amino acid sequence of a variant human TRPA1 channel protein (Mutant 7) is presented below:

Clone 63-Mutant 7
(SEQ ID NO: 7)
MKRSLRKMWRPGEKKEPQGVVYEDVPDDTEDFKES
LKVVFEGSAYGLONFNKQKKLKRCDDMDTFF----
--DYGNTPLHCAVEKNQIESVKFLLSRGANPNLRN
FNMMAPLHIAVQGMNNEVMKVLLEHRTIDVNLEGE
NGNTAVIIACTTNNSEALQILLKKGAKPCKSNKWG
CFPIHQAAFSGSKECMEIILRFGEEHGYSRQLHIN
FMNNGKATPLHLAVQNGDLEMIKMCLDNGAQIDPV
EKGRCTAIHFAATQGATEIVKLMISSYSGSVDIVN
TTDGCHETMLHRASLFDHHELADYLISVGADINKI
DSEGRSPLILATASASWNIVNLLLSKGAQVDIKDN
FGRNFLHLTVQQPYGLKNLRPEFMQMQQIKELVMD
EDNDGCTPLHYACRQGGPGSVNNLLGFNVSIHSKS
KDKKSPLHFAASYGRINTCQRLLQDISDTRLLNEG
DLHGMTPLHLAAKNGHDKVVQLLLKKGALFLSDHN
GWTALHHASMGGYTQTMKVILDTNLKCTDRLDEDG
NTALHFAAREGHAKAVALLLSHNADIVLNKQQASF
LHLALHNKRKEVVLTIIRSKRWDECLKIFSHNSPG
NKCPITEMIEYLPECMKVLLDFCMLHSTEDKSCRD
YYIEYNFKYLQCPLEFTKKTPTQDVIYEPLTALNA
MVQNNRIELLNHPVCKEYLLMKWLAYGFRAHMMNL
GSYCLGLIPMTILVVNIKPGMAFNSTGIINETSDH
SEILDTTNSYLIKTCMILVFLSSIFGYCKEAGQIF
QQKRNYFMDISNVLEWIIYTTGIIFVLPLFVEIPA
HLOWQCGAIAVYFYWMNFLLYLQRFENCGIFIVML
EVILKTLLRSTVVFIFLLLAFGLSFYILLNLQDPF
SSPLLSIIQTFSMMLGDINYRESFLEPYLRNELAH
PVLSFAQLVSFTIFVPIVLMNLLIGLAVGDIAEVQ
KHASLKRIAMQVELHTSLEKKLPLWFLRKVDQKST
IVYPNKPRSGGMLFHIFCFLFCTGEIRQEIPNADK
SLEMEILKQKYRLKDLTFLLEKQHELIKLIIQKME
IISETEDDDSHCSFQDREKKEQMEQRNSRWNTVLR
AVKAKTHHLEPFCYENE

In the above amino acid sequence of Clone 63-Mutant 7, the amino acid residues between amino acids 67 and 95 of the mutant polypeptide are deleted compared with the amino acid residues in this region of the hsTRPA1 WT Clone 63 amino acid sequence (FIG. 27). In an embodiment, an amino acid sequence having at least 85%, at least 88%, at least 90%, at least 91%, at least 92%, at least 93%, at least 94%, at least 95%, at least 96%, at least 97%, at least 98%, at least 99%, or 100% identity to the amino acid sequence of Mutant 7 above is encompassed.

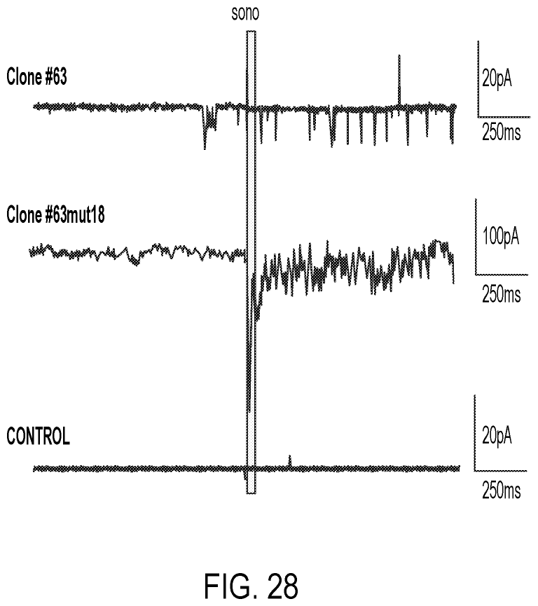

The wildtype (WT) hsTRPA1 Clone 63 TRPA1 channel polypeptide and the Mutant 18 (also called SonoChannel-1 herein) hsTRPA1 channel polypeptide were recombinantly expressed in excitable HEK cells. The electrophysiological responses of the membrane-expressed proteins versus control (e.g., vector expressing green fluorescent protein (GFP) control) were monitored using a patch clamp assay (20 pA, 250 ms), (FIG. 28). Significant electrophysiological responses to ultrasound were detected in HEK cells expressing the WT (Clone 63) or mutant (Mutant 18) TRPA1 channel polypeptides.

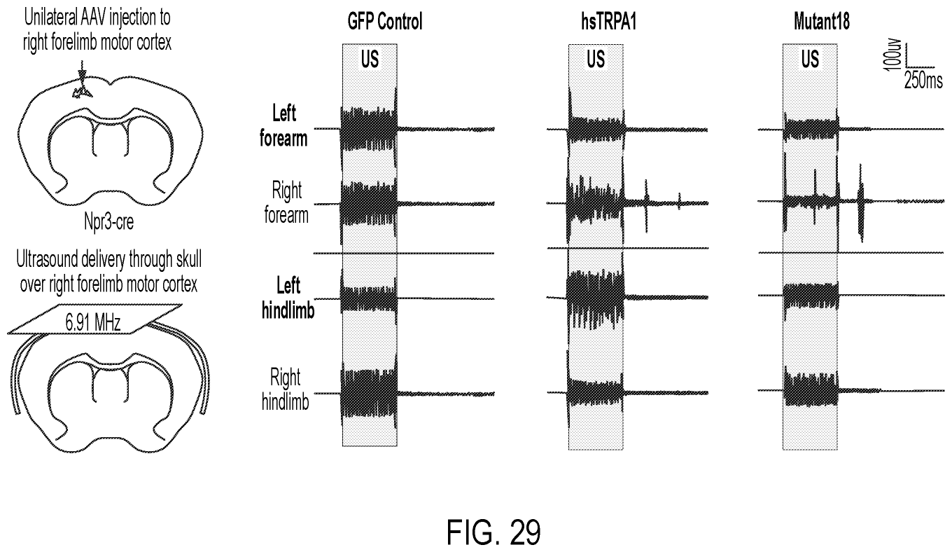

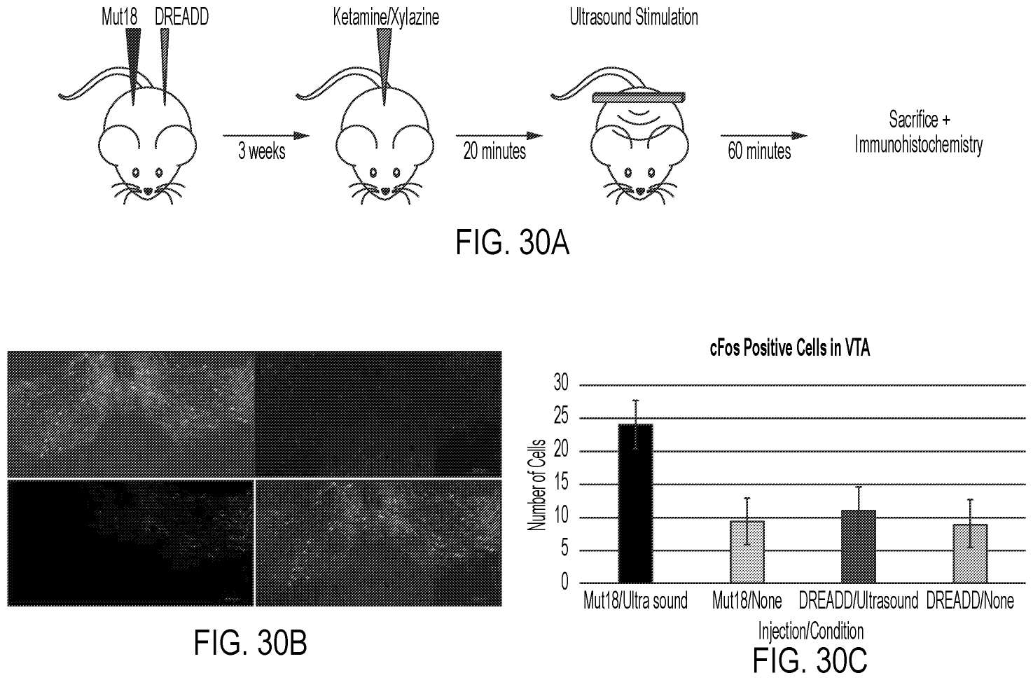

The ability of the Mutant 18 (SonoChannel-1) TRPA1 polypeptide to function in vivo was assessed. AAV containing polynucleotides encoding either WT Clone 63 polypeptide or mutant 18 (SonoChannel-1) was injected into the right forelimb motor cortex to allow for expression of the non-native channel proteins in motor cortical neurons that drive movement in right forelimb. Ultrasound was delivered through the skill over the right forelimb motor cortex. Ultrasound stimulation was expected to affect electrical responses in the right forelimb muscles (FIG. 29). As shown, expressing Mutant 18 TRPA1 channel polypeptide in dopaminergic neurons in the ventral tegumental area rendered them sensitive to ultrasound (FIGS. 30A-30C).

### Example 7: Materials and Methods

Materials and methods used in the above-described Examples are set forth herein below.
Animal husbandry. Studies were performed using a total of 50 adult mice including both males and females. Animals were group housed in an American Association for the Accreditation of Laboratory Animal Care approved vivarium on a 12-hour light/dark cycle, and all protocols were approved by the Institutional Animal Care and Use Committee of the Salk Institute for Biological Studies. Food and water were provided ad libitum, and nesting material was provided as enrichment. Colonies of C57Bl6/J (JAX #000664); Npr3-cre (Daigle, T. L. et al., Cell 174, 465-480 e422, doi:10.1016/j.cell.2018.06.035 (2018)), (JAX #031333); and TRPA1 knockout (Kwan, K. Y. et al., Neuron 50, 277-289, doi:10.1016/j.neuron.2006.03.042 (2006)), (JAX #006401) mice were maintained for experiments.
HEK cell culture and transfection. HEK cells expressing human αvβ3 integrin were cultured in DMEM supplemented with 10% FBS and 20 mM glutamine in a 5% CO₂ incubator. A stable calcium reporter line was generated with a GCaMP6f lentivirus (Cellomics Technology PLV-10181-50) followed by FACS sorting. For screening experiments and characterization of each candidate channel, GCaMP6f-expressing HEK cells were seeded on 12-well cell culture plates with 18 mm glass coverslips coated with PDL (10 μg/μl; Sigma-Aldrich P6407) for 1-2 hours. Coverslips were washed with Milli-Q water and cells were seeded at a density of 250000 cells/well. 24 hours after plating, cells were transfected with Lipofectamine LTX Reagent (ThermoFisher 15338100) according to the manufacturer's protocol, using 500 ng DNA of the clone of interest for each well. Cells were kept at 37° C. for an additional 24 hours before imaging on the ultrasound stimulation setup.
Mouse primary embryonic neuron culture. For WT primary neuron culture, timed pregnant C57Bl/6 female mice were ordered for E18 cortical dissociation (Charles River: 027). For TRPA1 knockout neuron culture, female TRPA1−/− (JAX #006401) dams were injected with luteinizing hormone releasing hormone (Sigma-Aldrich, L8008) 5 days before being paired with −/− males overnight. Pregnant dams were sacrificed and the E18 embryos were collected for cortical dissociation.
Mouse primary neuronal cultures were prepared from cortices isolated from embryonic day 18 (E18) mice, following the protocol described in Hilgenberg, L. G. & Smith, M. A. Preparation of dissociated mouse cortical neuron cultures. J Vis Exp, 562, doi:10.3791/562 (2007). Neurons were plated in 12-well culture plates with 18 mm PDL-coated coverslips (Neuvitro Corporation GG-18-PDL) at a concentration of 600-900 k cells/well. Neurons were then incubated at 37° C., 5% CO₂, with half media changes every 2-3 days with Neurobasal (ThermoFisher #21103049 supplemented with Primocin (InvivoGen #ant-pm-1), B-27 (ThermoFisher #17504044) and GlutaMAX (ThermoFisher #35050061). For calcium imaging experiments, cells were infected with AAV9-hSyn-GCaMP6f (Addgene #100837-AAV9) at day in vitro 3 (DIV3) and half media change was performed the next day.

Neurons infected with GCaMP6f as stated above were infected with AAV9-hSyn-Cre (Addgene #105553-AAV9) and AAV9-hSyn-TRPA1-myc-DIO (Salk GT3 core) at DIV4 and half media change was performed the next day. Cultures were incubated at 37° C., 5% CO₂ until DIV10-12 and then imaged using the same equipment as for HEK cell experiments.

Rat primary neuron culture. Rat primary neuronal cultures were prepared from rat pup tissue at embryonic (E) day 18 (E18) containing combined cortex, hippocampus and ventricular zone. The tissue was obtained from BrainBits (Catalogue #: SDEHCV) in Hibernate-E medium and used the same day for dissociation following the company's protocol. Briefly, tissue was incubated in a solution of Papain (BrainBits PAP) at 2 mg/mL for 30 min at 37° C. and dissociated in Hibernate-E for one minute using one sterile 9″ silanized Pasteur pipette with a fire polished tip. The cell dispersion solution was centrifuged at 1100 rpm for 1 minute, and the pellet was resuspended with 1 mL NbActiv1 (BrainBits NbActiv1 500 mL). Cell concentration was determined using a hemocytometer, and neurons were plated in 12-well culture plates with 18-mm PDL-coated coverslips (Neuvitro Corporation GG-18-PDL) at a concentration of 1.3 million cells/well. Neurons were then incubated at 37° C., 5% CO₂, performing half media changes every 3-4 days with fresh NbActiv1 supplemented with PRIMOCIN™ (InvivoGen ant-pm-1). Neurons infected with GCaMP6f as stated above were infected with AAV9-hSyn-Cre (Addgene #105553-AAV9) and AAV9-hSyn-TRPA1-myc-DIO (Salk GT3 core) at DIV4, and half media changes were performed the next day. Cultures were incubated at 37° C., 5% CO₂ until DIV10-12 and were used in electrophysiology experiments.
Ultrasound transducer: A set of custom-made single crystalline 127.68 Y-rotated X-propagating lithium niobate transducers operating in the thickness mode were used, as described in Collignon, S. et al., Advanced Functional Materials 28, 1704359 (2018). The fundamental frequency was measured to be 6.91 MHz using non-contact laser Doppler vibrometry (Polytec, Waldbronn, Germany), The devices were diced to 12 mm×12 mm and built into the in vitro test setup. The transducers were coated with a conductive layer of Au with a thickness of 1 μm with 20 nm of Ti acting as an adhesion layer. A DC sputtering (Denton 635 DC Sputtering system) process was used to coat 4″ wafers in an inert gas environment with a 2.3 m Torr pressure and rotation speed of 13 rpm, at a deposition rate of 1.5 A/s for Ti and 7A/s for Au. Devices were diced to size using an automated dicing saw (DISCO 3220) and the resonance frequency was verified using non-contact laser Doppler vibrometry
Imaging rig for ultrasound stimulation. For the 2D setup used, an existing upright epi-fluorescent Zeiss microscope was upgraded to perform a monolayer two-dimensional screen. For this application. the custom-made 12×12 mm lithium niobite (LiNbO₃) transducer placed in a heated stage fixture underneath the cell chamber was used. Stimulus frequency and duration was controlled by a waveform generator (Keysight 33600A Series), and pressure was controlled through a 300-W amplifier (VTC2057574, Vox Technologies, Richardson, Tex.). Simultaneous calcium imaging was performed using a 40× water dipping objective at 16.6 frames per second with an Orca Flash 4.0 camera and a GFP filter.
Candidate channel screen. A library of candidate channels was generated, which was initially based on a literature survey of naturally occurring ion channels and other membrane proteins were suggested to display mechanosensitive or ultrasound sensitive properties. From this initial list, related channels and variants from different species were selected, resulting in a final set of 191 proteins (Table 1 supra). Each channel was cloned into a custom bicistronic pcDNA3.1(+) vector using a porcine teschovirus-1 2A self-cleaving peptide (p2A) sequence, expressing the channel and the fluorescent protein dTomato under a human cytomegalovirus (CMV) promoter. All plasmids were generated by Genscript Biotech (New Jersey, United States).
In vitro pharmacology. For inhibition of TRPA1, cells were incubated with the antagonist HC030031, (Oh, S. J. et al., Curr Biol 29, 3386-3401 e3388, doi:10.1016/j.cub.2019.08.021 (2019)), (4004 in DMSO; Cayman Chemicals #11923) for 45 minutes before stimulation. For activation of TRPA1, N-Methylmaleimide (NMM), (Oh, S. J. et al., Curr Biol 29, 3386-3401 e3388, doi:10.1016/j.cub.2019.08.021 (2019)), (10004 in DMSO Sigma-Aldrich #389412) or allyl isothiocyanate (AITC), (Raisinghani, M. et al., Am J Physiol Cell Physiol 301, C587-600, oi:10.1152/ajpcell.00465.2010 (2011)), (3004 in DMSO; Sigma-Aldrich #377430) was used. For activation of Piezol, yoda-1, (Syeda, R. et al., eLife 4, doi:10.7554/eLife.07369 (2015)), (1004 in DMSO; Tocris #5586), was used. For activation of TRPV1 capsaicin (Chu, Y., et al., Sci Rep 10, 8038, doi:10.1038/s41598-020-64584-2 (2020)), (3 μM in DMSO; Sigma-Aldrich #M2028), was used. The final concentration of DMSO in the external solution was 0.1% or lower for all groups; which was also used as vehicle control. For cytoskeleton experiments, nocodazole (5 μM; Tocris, #1228), jasplakinolide (20004; ThermoFisher #J7473), paclitaxel (600 nM; Sigma-Aldrich #T7191), cytochalasin D (504; Cayman Chemicals, #11330) or latrunculin A (1 μM; Cayman Chemicals, #10630) in 0.1% DMSO were added to the culture medium 45 minutes prior to imaging. For pharmacology in primary neurons, the TRPV1 antagonist, A784168, (Cui, M. et al., J Neurosci 26, 9385-9393, doi:10.1523/JNEUROSCI.1246-06.2006 (2006)), (2004; Tocris, #4319, 45 minute incubation) in 0.1% DMSO, BAPTA, (Hofer, A. M., J Cell Sci 118, 855-862, doi:10.1242/jcs.01705 (2005)), (3004; Invitrogen, #B1204, 45 minute incubation) directly dissolved in culture medium, and TTX, (Lee, C. H. and Ruben, P. C., Channels (Austin) 2, 407-412, doi:10.4161/chan.2.6.7429 (2008)), (1804; tetrodotoxin citrate; Tocris #1069, 5 minute incubation, where TTX-R channels were also inhibited) were used.
Sequence collection and annotation: Ten TRPA1 peptide sequences were obtained from the National Center for Biotechnology Information (NCBI) RefSeq database for human (Homo sapiens; NCBI Taxonomy 9606; RefSeq XP_016869435.1), mouse (Mus musculus; NCBI Taxonomy 10090; RefSeq NM_177781), beaver (Castor canadensis; NCBI Taxonomy 51338; RefSeq XP_020010675.1), alpaca (Vicugna pacos; NCBI Taxonomy 30538; RefSeq XP_006202494.1), donkey (Equus asinus; NCBI Taxonomy 9793; RefSeq XP_014709261.1), bat (Eptesicus fuscus; NCBI Taxonomy 29078; RefSeq XP_008148609.1), alligator (Alligator mississippiensis; NCBI Taxonomy 8496; RefSeq XP_006277080.1), snake (Notechis scutatus; NCBI Taxonomy 8663; RefSeq XP_026545023.1), molly (Poecilia formosa; NCBI Taxonomy 48698; RefSeq XP_007554661.1), and zebrafish (Danio rerio; NCBI Taxonomy 7955; RefSeq NP_001007066.1). The human TRPA1 sequence was also obtained from the UniProtKB database and aligned against the human TRPA1 RefSeq sequence to confirm that the sequences were identical. Uniprot coordinates of major domains and features for human TRPA1 were used to annotate the sequence in Geneious Prime (version 2020.1.2).
Phylogenetic analysis. A multiple sequence alignment of all ten TRPA1 sequences was generated using Geneious Prime MAFFT (version 7.450), (Katoh, K. and Standley, D. M., Molecular biology and evolution 30, 772-780 (2013)), with a BLOSOM 62 scoring matrix, gap open penalty of 1.53, and offset value of 0.123. A phylogenetic gene tree based on the MAFFT alignment was generated using Geneious Prime RAxML (version 8.2.11) (Stamatakis, A., Bioinformatics 30, 1312-1313 (2014)), with a GAMMA BLOSOM 62 protein model, bootstrapping using rapid hill-climbing with seed 1, starting with a complete random tree, and using the maximum likelihood search convergence criterion. The maximum likelihood tree was assessed and annotated in FigTree (version 1.4.4).
Consensus sequence and percent identity. Consensus sequences for the ten tested chordate, mammalian, and non-mammal alignments, each having hsTRPA1 as reference, were generated in Geneious Prime. The threshold for consensus was set to 65%, as this ensured contribution from both mammalian and non-mammal sequences for chordate, from rodents, ungulates, and bats for mammalian, and from reptiles and fishes for non-mammal alignments. Alignment and consensus sequences were annotated in Geneious Prime to highlight either agreement or disagreement of a given amino acid relative to human TRPA1. Percent identity of consensus sequence to human was calculated to quantify the degree of sequence conservation or divergence in the chordate, mammalian, non-mammalian species.
CRAC-CARC motif annotation. CRAC ([LV]X(1,5)YX(1,5)[RK]) and CARC ([RK]X(1,5)[YF]X(1,5)[LV]) motifs, as defined in (Fantini, J. and Barrantes, F. J., Front Physiol 4, 31, doi:10.3389/fphys.2013.00031 (2013) were annotated per TRPA1 sequence using the Geneious Prime EMBOSS 6.5.7 fuzzpro tool (Rice, P. et al., Trends Genet 16, 276-277, doi:10.1016/s0168-9525(00)02024-2 (2000)).
Constructs of ankyrin TRPA1 mutants. To generate mutant constructs, a PCR based approach was used. Bicistronic constructs co-expressing deletion mutants and dTom were synthesized by Genscript Biotech (New Jersey, United States). For ΔANK1, amino acids (aa) 67-95 (Ref seq. UniProtKB-O75762) were deleted, corresponding to nucleotide deletions 2641-2727 (Ref seq. XM_017013946.1). For the rest of constructs the aa and nucleotide deletions were as follows: ΔN-tip: aa deletions 1-61, nucleotide deletions 1-182; ΔN-tip (1-25), aa deletions 1-25, nucleotide deletions 1-75; CRAC mutant, swapping Tyr (Y)785 to Ser (S), nucleotides 2353-2355 (TAC) to TCG; alligator N-tip, swapping aa 1-66 from hsTRPA1 to first 66 residues from amTRPA1; zebrafish N-tip, swapping aa 1-59 from hsTRPA1 to first 59 residues from drTRPA1.
In vitro electrophysiology. A stable line of HEK cells expressing Nav1.3 and Kir2.1 (Ex-HEK, ATCC CRL-3269) were cultured on 18 mm round coverslips at a seeding density of ˜300 k cells/well in a tissue-culture treated 12-well plate. Cells were transiently transfected with a custom plasmid (Genscript) expressing hsTRPA1 and dTom fluorescent reporter as for screening experiments, 18-24 h post seeding. Cells underwent a media change, were allowed to recover, and then were used for recordings 18-24 h after transfection. Coverslips were transferred to a custom machined acrylic stage containing a bath of external solution (NaCl (140 mM), KCl (4 mM), MgCl₂ (2 mM), Glucose (5 mM) and HEPES (10 mM)) with an osmolarity of ˜290 mOsm. Patch pipettes were pulled on a Sutter puller model P-97 programmed to give 4-6 MΩ tips from filamented borosilicate glass (o.d. 1.5 mm, i.d. 0.86 mm). The internal solution was CsF or KF based and obtained from Nan]i[on (#08 3008, #08 3007). An Olympus 40× water dipping lens with 0.8 NA was used in combination with a (QImaging OptiMOS) cMOS camera to visualize cells with Köhler or fluorescent illumination. dTom signal was used to confirm hsTRPA1 expression in HEK cells. Electrical signals were acquired using Axon Instruments Multiclamp 700B amplifier and digitized with Digidata using pClamp acquisition and control software. Gap free recordings were conducted (typically holding the membrane potential at −70 mV) while delivering 100 ms pulses of ultrasound. The ultrasound delivery rig used for patch clamp experiments was the same as that used for imaging experiments. Briefly, waveforms were programmed using an arbitrary function generator (Keysight Technologies) connected via BNC to an amplifier (VTC2057574, Vox Technologies). Military communications grade BNC cables (Federal Custom Cable) were used to ensure impedance matching in the experimental systems and to reduce electrical interference. The amplifier was connected to a custom-made lithium niobate transducer as described herein mounted on a dove-tail sliding arm, and coupled to the bottom of the recording chamber with ultrasound gel. The center of the transducer was left uncoated with gold in order to permit bright-field light to reach the sample, allowing for the alignment of optics and obtaining even illumination for DIC imaging. Recordings were carried out in response to peak negative pressures ranging from 0.2-0.25 MPa, as access resistance could not be maintained when high pressures were delivered. Cell attached Ex-HEK-GCaMP cells-maintained membrane resistances between 0.5 and 3 GΩ. Patch-clamp experiments conducted on primary dissociated cortical neurons followed a modified protocol. Briefly, neurons were allowed to mature for 11-14 days in vitro prior to recording. Compared to HEK cells, neuron somatic morphology was better suited for whole-cell recording configuration. Both voltage-clamp (VC) and current clamp (CC) recordings were conducted. Upon successful whole-cell access, baseline gap-free recordings in CC or VC trials were obtained. Ultrasound stimulation parameters followed the same protocol as was used for the HEK cell recordings. For primary cortical neuron experiments, access resistance during successful whole-cell recordings was maintained between 10 to 25 MΩ.
Viruses. pAAV.Syn.DIO.hsTRPA1-myc plasmid was custom made by GenScript. synP.DIO.EGFP.WPRE.hGH was a gift from Ian Wickersham (Addgene viral prep #100043-AAV9). pAAV.Syn.GCaMP6f.WPRE.SV40, (Chen, T. W. et al., Nature 499, 295-300, doi:10.1038/nature12354 (2013)), was a gift from Douglas Kim & GENIE Project (Addgene viral prep #100837-AAV9; http://n2t.net/addgene:100837; RRID:Addgene_100837). pENN.AAV.hSyn.Cre.WPRE.hGH was a gift from James M. Wilson (Addgene viral prep #105553-AAV9; http://n2t.net/addgene:105553; RRID:Addgene 105553). AAV9-hSyn-DIO-hsTRPA1-myc (GT3 Core at Salk Institute of Biological Studies) was injected at either 4E13 along with 1E12 AAV9-hSyn-DIO-GFP (Addgene #100043-AAV9) diluted in Hank's Balanced Salt Solution for injection. Adult male and female Npr3-cre mice (19-30 g) received 400 nL unilateral injections to the right motor cortex at AP 0.0 ML −1.0, AP+0.5 ML −1.0, AP+0.5 ML−1.5 at DV 0.5 (Ueno, M. et al., Cell reports 23, 1286-1300 e1287, doi:10.1016/j.celrep.2018.03.137 (2018)). Briefly, small holes were drilled (0.45 mm drill bit) into the skull over those coordinates, and virus was delivered through a pulled glass pipette at 2 nL/sec by a Nanoject iii (Drummond Scientific Company). Successful viral delivery was confirmed post-mortem via immunohistochemistry for GFP and/or the myc-tag.
Ca2+ imaging analysis. All image analysis was performed using custom scripts written as ImageJ Macros. Cells in the dTom channel were segmented and cell fluorescence over time in the GCaMP channel was measured and stored in csv files. Briefly, the script uses a gaussian filter on the dTomato channel and background subtraction, followed by auto thresholding and watershed segmentation. The plugin ‘Analyze particles’ was then used to extract counts. Calcium data were analyzed using custom Python scripts. Calcium signal was normalized as ΔF/F using a 6 s baseline for each ROI, and a peak detection algorithm with a fixed threshold of 0.25 was used to identify responsive cells after ultrasound stimulation, similar to the approach used by Oh, S. J. et al., Curr Biol 29, 3386-3401 e3388, doi:10.1016/j.cub.2019.08.021 (2019). For the screen, the number of cells showing a response to ultrasound was calculated as the total percent of responsive cells after 3 consecutive 90 second recordings on the same coverslip. The percent of transfected cells was calculated as the number of dTom positive cells/total number of cells per field of view imaged. To compare ultrasound response between clones, a generalized mixed model was used, fitting “response” as a Bernoulli response, “clone” as a fixed factor, and “cell” as a random effect. Pairwise comparisons were later performed using odds ratios and Tukey method, correcting for multiple comparisons.

Peak amplitude was calculated for each trace as the maximum GCaMP6f ΔF/F value during 60 s after ultrasound stimulation or pharmacological treatment for HEK cells, and 5 s for mouse primary neurons. For the AITC response curve in neurons, mean GCaMP6f ΔF/F up to 1.5 min after adding AITC to the medium was used instead of peak amplitude response. For latency and duration analysis in primary neurons, latency of calcium responses was measured as the time to reach 63% of the peak amplitude after stimulation, while width was calculated as the distance between 63% rise and 63% decay.

Immunocytochemistry. Cells were fixed with 4% paraformaldehyde (PFA) at room temperature for 15 minutes and subsequently were permeabilized using 0.25% Triton X-100 PBS with 5% horse serum. After incubation in blocking solution for 1 hour at room temperature, cells were incubated overnight at 4° C. with different primary antibodies: for HEK cells, a mouse monoclonal anti-TRPA1 antibody (1:1000, Santa Cruz Biotech #376495) was used, while a polyclonal anti-myc antibody (1:1000; Cell Signalling Tech #2272S) was used to detect the tagged channel in primary neuron cultures. Secondary antibody staining was performed at room temperature for 2 hours, followed by DAPI for 30 min. For myc, TSA amplification was performed to increase the signal. Co-localization to the cell membrane was determined via co-transfection and co-immunolabeling with EGFP-CAAX, (Madugula, V. and Lu, L., J Cell Sci 129, 3922-3934, doi:10.1242/jcs.194019 (2016)), which was a gift from Lei Lu (Addgene plasmid #86056; http://n2t.net/addgene:86056; RRID:Addgene_86056). For cytoskeleton immunolabeling experiments, fixed cells were incubated with anti-alpha-tubulin antibody (Sigma, #CBL270-I, 1:1000) or phalloidin-488 (ThermoFisher, #A12379, 1:500).
Rotarod. Mouse locomotor behaviour was evaluated on a Rotor-Rod (SD Instruments). Mice underwent a single day of training at a constant speed of 3 RPM to acclimate to the Rotor-Rod. The next day, mice were placed on a rod that started at 0 RPM and gradually increased to 30 RPM over a 5-minute period. The latency to fall off the rod was collected. Each mouse underwent 4 trials daily with a 20-minute inter-trial interval in which mice were returned to their cages. The latency to fall off was averaged across the three best trials. This procedure was repeated across 5 days. The experimenter was blinded as to the identity of groups.
Electromyography (EMG) experiments. EMG experiments were conducted between 2-4 weeks after viral injection. EMG data were collected under ketamine (100 mg/kg) and xylazine (10 mg/kg) anaesthesia from the right and left biceps brachii and right and left biceps femoris through fine wire electrodes (A-M Systems 790700) connected to a PowerLab and BioAmp (AD Instruments). Data were collected at 40 k/sec, bandpass filtered from 300 Hz to 1 kHz. Correct electrode placement was confirmed by positive EMG signal in response to pinch. The skin over the skull was opened, and the 6.91 MHz lithium niobate ultrasound transducer was coupled to the skull using ultrasound gel (Parker Aquasonic 100). Ultrasound stimuli (1, 10, 100 msecs durations) were administered at no less than 10 second intervals at intensities ranging from 0.35-1.05 MPa, intracortical pressure. Visual movement of the right fore or hindlimb in response to stimulation was noted, and EMG responses were analyzed for latency and duration. Due to the relatively large stimulus artefact from the ultrasound pulse, responses occurring during the ultrasound stimulus could not be reliably quantified. Therefore, only responses occurring after cessation of the stimulus were considered in the analyses described herein. The experimenter was blinded as to the group during both collection and analysis of the data.
US pressure and temperature measurements. Ultrasound pressure and temperature measurements were collected through ultrasound gel at the same position from the face of the lithium niobate transducer and within the brain tissue through the skull using a Precision Acoustics Fibre-Optic Hydrophone connected to a Tektronix TBS 1052B Oscilloscope and ThinkPad Ultrabook. To enable stereotaxic insertion into the brain, the Fibre-Optic Hydrophone probe was carefully threaded through a glass capillary, allowing the tip to remain exposed. Cortical measurements were taken in ex-vivo cranial tissue in which the jaw and palate were removed to expose the base of the brain. Using the center of the hypothalamus as coordinates 0,0,0, the hydrophone was inserted at AP +1.2, ML 1.0 and lowered to a depth of −5.6 to approximate the location of the layer V motor cortex. The transducer was coupled to the skull via ultrasound gel, and temperature and pressure measurements were collected.
Immunohistochemistry and c-fos quantification. At the conclusion of the study, mice were perfused with 0.9% saline followed by 4% paraformaldehyde (PFA) through a peristaltic pump. Brain tissue was immediately collected and incubated in 4% PFA overnight before being changed to 30% sucrose. Tissue was then sectioned at 35 μM into tissue collection solution (glycerine, ethylene glycol, NaH₂PO₄, Na₂HPO₄) and stored at 4° C. For brain immunohistochemistry, brain sections from every ˜350 μM were immunolabeled for myc (1:500; Cell Signalling 2272S), c-fos (1:500; Encor RCPA-cfos), NeuN (1:1500; Synaptic Systems 226004), GFP (1:1000; AVES GFP-1010) and DAPI (1:1000). Tyramide amplification was used to enhance the myc and c-fos signals. Briefly, tissue was incubated for 30 min in H₂O₂, blocked for 1 hr in PBST plus 5% horse serum, and then incubated overnight with primary antibodies. The next day, tissue was incubated for 3 hours at room temperature with biotinylated donkey anti-rabbit antibody (1:500, Jackson Immunoresearch 711-065-152), and then washed, incubated with ABC (Vector Labs PK-4000) for 30 min, washed, incubated with tyramide, washed and incubated with streptavidin conjugated antibody along with secondary antibodies (Thermo Fisher Scientific and Jackson Immunoresearch) directed to other antigens of interest for 3 hrs at room temperature or overnight at 4° C. Tissue was then mounted onto glass slides and cover slipped with Prolong Gold Antifade mounting medium (Thermo Fisher Scientific). Imaging for quantification of c-fos and myc expression was conducted at 10× on a Zeiss Axio Imager.M2 connected to an OrcaFlash 4.0 C11440 camera. High quality images depicting myc and fos co-localization with GFP were taken on a Zeiss Airyscan 880 microscope. Imaging of whole brain sections was performed at 10× on an Olympus VS-120 Virtual Slide Scanning Microscope.

Quantification of c-fos and GFP positive neurons was conducted in FIJI (Schindelin, J. et al., Nat Methods 9, 676-682, doi:10.1038/nmeth.2019 (2012)),using manual cell counting. c-fos puncta were excluded if they did not colocalize with DAPI. Myc+ GFP+ neurons were also quantified using manual cell counting in FIJI. Only GFP+ cell bodies that were completely filled with myc immunolabeling were considered to be myc+. The experimenter was blinded as to the experimental condition during quantification.

BaseScope. Adult TRPA1 knockout (JAX #006401) and wild-type C57Bl6/J (JAX #000664) were perfused with 0.9% saline. A WT C57/B16 E18 mouse embryo was also collected from a cohort of embryos slated for dissociation for use in in vitro experiments. Brains and lumbar dorsal root ganglia (DRG) were extracted and immediately frozen in OCT. Fresh frozen sections (10 μm) were direct mounted and slides were stored at −80° C. overnight. A custom BaseScope probe (BA-Mm-Trpa1-3zz-st, ACD-Bio Probe Design #: NPR-0003309) targeting 2602-2738 of mouse TRPA1 (NM_177781.5. GTGATTTTT AAAACATTGC TGAGATCGAC CGGAGTGTTT ATCTTCCTCC TACTGGCTTT TGGCCTCAGC TTTTATGTTC TCCTGAATTT CCAAGATGCC TTCAGCACCC CATTGCTTTC CTTAATCCAG ACATTCAG) (SEQ ID NO: 8) was used. This region was chosen because it is deleted in the TRPA1 knockout mouse. Tissues were also probed with positive (Ppib, ACD #701071) and negative control (DapB, ACD #701011) probes, which gave the expected results in all experiments. DRG tissue was used as positive control for the TRPA1 probe, as small-diameter DRG neurons are known to express TRPA1. DRGs and cortices from TRPA1 knockout mice were used as a negative control for the TRPA1 probe.
Blood brain barrier (BBB) experiments. Mice received retro-orbital injections of 10 kDa fluorescein isothiocyanate-dextran (Sigma FD10S) at 150 mg/mL in saline. Positive control mice immediately received a cortical stab wound with a 27 g needle through a small hole drilled at AP 0, ML −1, DV −0.5. Ultrasound-treated mice had their scalp opened, and the ultrasound transducer was coupled over the left cortex with ultrasound gel. The transducer delivered 100 ms stimuli at 0.88 MPa every 10 seconds for 1 hour. Sham-treated mice underwent the same procedure, except that the transducer was not turned on. A cohort of mice that did not receive dextran injection or ultrasound was also collected for use as a negative control, and all values were normalized to this cohort. (n=4-5 per group, evenly split between male and female). Mice were perfused 70 minutes after cortical injection or start of the ultrasound treatment. One ultrasound-treated mouse had to be omitted from the data set due to inadequate perfusion. Brain sections were sectioned at 35 μM and processed as floating sections. After blocking, sections were incubated with 647 donkey anti-mouse IgG and DAPI. Images were collected on an Olympus Virtual Slide Scanner (VS-120), using the same settings across all groups. Fluorescein isothiocyanate-dextran 488 and 647-mouse IgG were quantified as mean intensity in left and right cortex from each sample using FIJI. All values were normalized to fluorescent values obtained from samples that received neither dextran nor ultrasound.
Quantification and statistical analyses. Statistical analyses were performed in GraphPad Prism and R. All statistical tests in the described studies herein were two-tailed. Single-variable comparisons were made with Mann-Whitney test. Group comparisons were made using either analysis of variance (ANOVA) followed by Tukey-Kramer post-hoc analysis or non-parametric Kruskal-Wallis test followed by Dunn's post-hoc analysis. The ROUT method in GraphPad Prism with a q=0.2% was used to identify and exclude outliers. Statistics used to analyze calcium imaging data are described in Methods. No statistical methods were used to predetermine sample sizes for single experiments. The code used to analyze calcium imaging data are available at https://github.com/shreklab/Duque-Lee-Kubli-Tufail2020.git.

1. A method of stimulating a cell, the method comprising contacting a TRPA1 polypeptide-expressing cell with ultrasound, thereby stimulating the cell.

2. The method of claim 1, wherein the TRPA1 polypeptide or functional fragment thereof has at least about 85% identity to a TRPA1 polypeptide having the sequence of NCBI Reference Sequence: XP_016869435.1 (SEQ ID NO: 1) or selected from the group consisting of:
(SEQ ID NO: 4)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVO
GMNNEVMKVLLEHRTIDVNLEGENGNTAVIIACTT
NNSEALQILLKKGAKPCKSNKWGCFPIHQAAFSGS
KECMEIILRFGEEHGYSROLHINFMNNGKATPLHL
AVONGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQP
LHFAASYGRINTCORLLODISDTRLLNEGDLHGMT
PLHLAAKNGHDKVVQLLLKKGALFLSDHNGWTALH
HASMGGYTQTMKVILDTNLKCTDRLDEDGNTALHF
AAREGHAKAVALLLSHNADIVLNKQQASFLHLALH
NKRKEVVLTIIRSKRWDECLKIFSHNSPGNKCPIT
EMIEYLPECMKVLLDFCMLHSTEDKSCRDYYIEYN
FKYLQCPLEFTKKTPTQDVIYEPLTALNAMVONNR
IELLNHPVCKEYLLMKWLAYGFRAHMMNLGSYCLG
LIPMTILVVNIKPGMAFNSTGIINETSDHSEILDT
TNSYLIKTCMILVFLSSIFGYCKEAGQIFQQKRNY
FMDISNVLEWIIYTTGIIFVLPLFVEIPAHLOWQC
GAIAVYFYWMNFLLYLORFENCGIFIVMLEVILKT
LLRSTVVFIFLLLAFGLSFYILLNLQDVGDIAEVQ
KHASLKRIAMQVELHTSLEKKLPLWFLRKVDQKST
IVYPNKPRSGGMLFHIFCFLFCTGEIRQEIPNADK
SLEMEILKQKYRLKDLTFLLEKQHELIKLIIQKME
IISETEDDDSHCSFQDRFKKEQMEQRNSRWNTVLR
AVKAKTHHLEP;
(SEQ ID NO: 5)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSROLHINFMNNGKATPLHL
AVONGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQP
LHFAASYGRINTCQRLLQDISDTRLLNEGDLHGMT
PLHLAAKNGHDKVVQLLLKKGALFLSDHNGWTALH
HASMGGYTQTMKVILDTNLKCTDRLDEDGNTALHF
AAREGHAKAVALLLSHNADIVLNKQQASFLHLALH
NKRKEVVLTIIRSKRWDECLKIFSHNSPGNKCPIT
EMIEYLPECMKVLLDFCMLHSTEDKSCRDYYIEYN
FKYLQCPLEFTKKTPTQDVIYEPLTALNAMVQNNR
IELLNHPVCKEYLLMKWLAYGFRAHMMNLGSYCLG
LIPMTILVVNIKPGMAFNSTGIINETSDHSEILDT
TNSYLIKTCMILVFLSSIFGYCKEAGQIFQQKRNY
FMDISNVLEWIIYTTGIIFVLPLFVEIPAHLQWQC
GAIAVYFYWMNFLLYLQRFENCGIFIVMLEVILKT
LLRSTVVFIFLLLAFGLSFYILLNLQDPFSSPLLS
IIQTFSMMLGDINYRESFLHPYLRNELAHPVLSFA
QLVSFTIFVPIVLMNLLIGLAVGDIAEVQKHASLK
RIAMQVELHTSLEKKLPLWFLRKVDQKSTIVYPNK
PRSGGMLFHIFCFLFCTGEIRQEIPNADKSLEMEI
LKQKYRLKDLTFLLEKQHELIKLIIQKMEIISETE
DDDSHCSFQDRFKKEQMEQRNSRWNTVLRAVKAKT
HHLEP;
(SEQ ID NO: 6)
MKRSLRKMWRPGEKKEPQGVVYEDVPDDTEDFKES
LKVVFEGSAYGLQNFNKQKKLKRCDDMDTFFLHYA
AAEGQIELMEKITRDSSLEVLHEMDDYGNTPLHCA
VEKNQIESVKFLLSRGANPNLRNFNMMAPLHIAVQ
GMNNEVMKVLLEHRTIDVNLEGENGNTAVIIACTT
NNSEALQILLKKGAKPCKSNKWGCFPIHQAAFSGS
KECMEIILRFGEEHGYSRQLHINFMNNGKATPLHL
AVQNGDLEMIKMCLDNGAQIDPVEKGRCTAIHFAA
TQGATEIVKLMISSYSGSVDIVNTTDGCHETMLHR
ASLFDHHELADYLISVGADINKIDSEGRSPLILAT
ASASWNIVNLLLSKGAQVDIKDNFGRNFLHLTVQP
LHFAASYGRINTCQRLLQDISDTRLLNEGDLHGMT
PLHLAAKNGHDKVVQLLLKKGALFLSDHNGWTALH
HASMGGYTQTMKVILDTNLKCTDRLDEDGNTALHF
AAREGHAKAVALLLSHNADIVLNKQQASFLHLALH
NKRKEVVLTIIRSKRWDECLKIFSHNSPGNKCPIT
EMIEYLPECMKVLLDFCMLHSTEDKSCRDYYIEYN
FKYLQCPLEFTKKTPTQDVIYEPLTALNAMVONNR
IELLNHPVCKEYLLMKWLAYGFRAHMMNLGSYCLG
LIPMTILVVNIKPGMAFNSTGIINETSDHSEILDT
TNSYLIKTCMILVFLSSIFGYCKEAGQIFQQKRNY
FMDISNVLEWIIYTTGIIFVLPLFVEIPAHLQWQC
GAIAVYFYWMNFLLYLQRFENCGIFIVMLEVILKT
LLRSTVVFIFLLLAFGLSFYILLNLQDPFSSPLLS
IIQTFSMMLGDINYRESFLEPYLRNELAHPVLSFA
QLVSFTIFVPIVLMNLLIGLAVGDIAEVQKHASLK
RIAMQVELHTSLEKKLPLWFLRKVDQKSTIVYPNK
PRSGGMLFHIFCFLFCTGEIRQEIPNADKSLEMEI
LKQKYRLKDLTFLLEKQHELIKLIIQKMEIISETE
DDDSHCSFQDRFKKEQMEQRFCYENE;
(SEQ ID NO: 7)
MKRSLRKMWRPGEKKEPQGVVYEDVPDDTEDFKES
LKVVFEGSAYGLQNFNKQKKLKRCDDMDTFFDYGN
TPLHCAVEKNQIESVKFLLSRGANPNLRNFNMMAP
LHIAVQGMNNEVMKVLLEHRTIDVNLEGENGNTAV
IIACTTNNSEALQILLKKGAKPCKSNKWGCFPIHQ
AAFSGSKECMEIILRFGEEHGYSRQLHINFMNNGK
ATPLHLAVONGDLEMIKMCLDNGAQIDPVEKGRCT
AIHFAATQGATEIVKLMISSYSGSVDIVNTTDGCH
ETMLHRASLFDHHELADYLISVGADINKIDSEGRS
PLILATASASWNIVNLLLSKGAQVDIKDNFGRNFL
HLTVQQPYGLKNLRPEFMQMQQIKELVMDEDNDGC
TPLHYACRQGGPGSVNNLLGFNVSIHSKSKDKKSP
LHFAASYGRINTCQRLLQDISDTRLLNEGDLHGMT
PLHLAAKNGHDKVVQLLLKKGALFLSDHNGWTALH
HASMGGYTQTMKVILDTNLKCTDRLDEDGNTALHF
AAREGHAKAVALLLSHNADIVLNKQQASFLHLALH
NKRKEVVLTIIRSKRWDECLKIFSHNSPGNKCPIT
EMIEYLPECMKVLLDFCMLHSTEDKSCRDYYIEYN
FKYLQCPLEFTKKTPTQDVIYEPLTALNAMVONNR
IELLNHPVCKEYLLMKWLAYGFRAHMMNLGSYCLG
LIPMTILVVNIKPGMAFNSTGIINETSDHSEILDT
TNSYLIKTCMILVFLSSIFGYCKEAGQIFQQKRNY
FMDISNVLEWIIYTTGIIFVLPLFVEIPAHLOWQC
GAIAVYFYWMNFLLYLQRFENCGIFIVMLEVILKT
LLRSTVVFIFLLLAFGLSFYILLNLQDPFSSPLLS
IIQTFSMMLGDINYRESFLEPYLRNELAHPVLSFA
QLVSFTIFVPIVLMNLLIGLAVGDIAEVQKHASLK
RIAMQVELHTSLEKKLPLWFLRKVDQKSTIVYPNK
PRSGGMLFHIFCFLFCTGEIRQEIPNADKSLEMEI
LKQKYRLKDLTFLLEKQHELIKLIIQKMEIISETE
DDDSHCSFQDRFKKEQMEQRNSRWNTVLRAVKAKT
HHLEPFCYENE;

3-11. (canceled)

12. A method of inducing cation influx in a cell, the method comprising:
(a) expressing a heterologous TRPA1 polypeptide or fragment thereof in a cell; and
(b) applying ultrasound to the cell, thereby inducing cation influx in the cell.

13-17. (canceled)

18. The method of claim 1, wherein the cell is muscle cell, cardiac muscle cell, neuron, motor neuron, sensory neuron, interneuron, or insulin secreting cell.

19. The method of claim 1, wherein the ultrasound frequency is about 0.8 MHz to about 4 MHz.

20. The method of claim 1, wherein the ultrasound frequency is about 6.91 MHz.

21. The method of claim 1, wherein the ultrasound comprises an ultrasonic wave comprising a focal zone of about 1 cubic millimeter to about 1 cubic centimeter.

22. The method of claim 1, wherein the method further comprises contacting the cell with a microbubble prior to applying ultrasound.

23. (canceled)

24. A method of treating a disease or disorder in a subject in need thereof, said method comprising:
(i) expressing in a cell of the subject a heterologous nucleic acid molecule encoding a TRPA1 polypeptide or fragment thereof; and
(ii) applying ultrasound to the cell, thereby treating the disease or disorder in the subject.

25. The method of claim 24, wherein the disease or disorder is a neurological disease or disorder.

26. The method of claim 25, wherein the neurological disease or disorder is selected from the group consisting of Parkinson Disease, depression, obsessive-compulsive disorder, chronic pain, epilepsy or cervical spinal cord injury.

27. The method of claim 24, wherein the disease or disorder is muscle weakness.

28-31. (canceled)

32. The method of claim 1, wherein the ultrasound stimulates or triggers a response by the TRPA1-expressing cell.

33. (canceled)

34. A non-naturally occurring TRPA1 polypeptide comprising the amino acid sequence of SEQ ID NO: 4, 5, 6, or 7.

35-37. (canceled)

38. A viral vector comprising a polynucleotide encoding a TRPA1 polypeptide or a functional fragment thereof.

39. The viral vector of claim 38, wherein the TRPA1 polypeptide or the functional fragment thereof comprises an amino acid sequence selected from the group consisting of SEQ ID NOs: 1-7, or a functional fragment thereof.

40. (canceled)

41. The viral vector of claim 38, wherein the vector is a lentiviral vector or an adeno-associated viral vector.

42. A cell comprising the TRPA1 polypeptide of claim 34.

43. A cell comprising the viral vector of claim 38.

44-45. (canceled)

46. A composition comprising the viral vector of claim 38.

47. (canceled)

48. A composition comprising the cell of claim 42.

49. (canceled)
